(19) United States
(12) Patent Application Publication
(10) Pub. No.: US 2024/0374562 A1
(43) Pub. Date: Nov. 14, 2024

(54) METHODS AND COMPOSITIONS COMPRISING A NITRITE-REDUCTASE PROMOTER FOR TREATMENT OF MEDICAL DISORDERS AND PRESERVATION OF BLOOD PRODUCTS

(71) Applicant: EpicentRx, Inc., La Jolla, CA (US)

(72) Inventors: Bryan T. ORONSKY, Los Altos Hills, CA (US); Jan SCICINSKI, Saratoga, CA (US); Susan KNOX, Stanford, CA (US); William FITCH, Palo Alto, CA (US); Frans A. KUYPERS, El Cerrito, CA (US); Marcel FENS, San Francisco, CA (US); Sandra LARKIN, Berkeley, CA (US); Pedro CABRALES, San Diego, CA (US); Chad BROUSE, San Diego, CA (US)

(73) Assignee: EpicentRx, Inc., La Jolla, CA (US)

(21) Appl. No.: 18/182,153

(22) Filed: Mar. 10, 2023

Related U.S. Application Data
(63) Continuation of application No. 16/284,035, filed on Feb. 25, 2019, now Pat. No. 11,660,286.
Application No. 16/284,035 is a continuation of application No. 14/349,010, filed on Apr. 1, 2014, now abandoned.
(60) Provisional application No. 61/544,375, filed on Oct. 7, 2011.

(51) Int. Cl. A61K 31/397; A01N 1/02; A61K 31/12; A61K 31/16; A61K 31/198; A61K 31/215; A61K 31/22; A61K 31/255; A61K 31/40; A61K 31/445; A61K 33/00; A61K 35/14; A61K 35/16; A61K 35/18; A61K 38/17; A61K 45/06; A61K 47/64; A61N 5/10

(52) U.S. Cl. CPC A61K 31/397; A01N 1/0226; A61K 31/12; A61K 31/16; A61K 31/198; A61K 31/215; A61K 31/22; A61K 31/255; A61K 31/40; A61K 31/445; A61K 33/00; A61K 35/14; A61K 35/16; A61K 35/18; A61K 38/1709; A61K 45/06; A61K 47/6445; A61N 5/10; Y02A 50/30

(57) ABSTRACT

The invention provides methods, compositions, and medical kits comprising a nitrite-reductase promoter, such as an allosteric modulator of hemoglobin, for use in treating medical disorders and preservation of blood products. In one aspect, the invention provides methods, compositions, and medical kits comprising an inorganic nitrite salt and a nitrite-reductase promoter, such as an allosteric modulator of hemoglobin, for use in treating medical disorders, such as cancer, cardiovascular disorders, ischemic conditions, hemolytic conditions, and bacterial infections. Exemplary inorganic nitrite salts include sodium nitrite and arginine nitrite. Exemplary allosteric modulators of hemoglobin described herein include alkyl-substituted and acyl-substituted di-nitroheterocycles.

### CROSS REFERENCE TO RELATED APPLICATIONS

This application is a continuation of U.S. Non-Provisional patent application Ser. No. 16/284,035, filed on Feb. 25, 2019, which is a continuation of U.S. Non-Provisional patent application Ser. No. 14/349,010, filed on Apr. 1, 2014, which is a U.S. National Stage of PCT/US12/58964, filed on Oct. 5, 2012, which claims the benefit of and priority to U.S. Provisional Patent Application Ser. No. 61/544,375, filed Oct. 7, 2011, the contents of which are hereby incorporated by reference.

### FIELD OF THE INVENTION

The invention provides methods, compositions, and medical kits comprising a nitrite-reductase promoter, such as an allosteric modulator of hemoglobin, for use in treating medical disorders and preservation of blood products.

### BACKGROUND

Cancer, cardiovascular disorders, ischemic conditions, and bacterial infections remain a significant health problem for people in many developed countries. The need for cancer treatments, for example, has prompted the United States National Cancer Institute to coordinate large-scale research efforts, impacting over six hundred universities, hospitals, and cancer centers located in the United States and over twenty foreign countries. Past research efforts have lead to significant advances in the detection, evaluation, and treatment of cancer, cardiovascular disorders, ischemic conditions, and bacterial infections. However, despite these developments, these medical conditions remain significant health problems for many patients.

According to current statistics, cancer is a leading cause of death worldwide. Approximately one million people are diagnosed with cancer each year in the United States, and approximately half a million cancer patients die annually despite the significant progress made during the last decade in the diagnosis and treatment of cancer. Leading types of cancer that affect a substantial number of patients include colon cancer, breast cancer, prostate cancer, and skin cancer. The need exists for improved drugs and therapeutic methods for treating cancer.

Cardiovascular disorders that affect a significant number of patients include atherosclerosis, arteriosclerosis, myocardial infarction, angina pectoris, cardiac failure, embolism, thrombus, and hypertension. Hypertension is particularly prevalent, with some estimates suggesting approximately twenty-five percent of the adult population worldwide being hypertensive. Although dietary and lifestyle changes may reduce blood pressure, medications are often necessary to reduce blood pressure to an acceptable level in hypertensive patients. Examples of anti-hypertensive drugs include angiotensin-converting enzyme (ACE) inhibitors, alpha blockers, angiotensin II receptor antagonists, beta blockers, calcium channel blockers, diuretics, and direct renin inhibitors. Without treatment, hypertensive patients can have a significantly higher risk of cardiovascular disorders and a reduced life expectancy. The need exists for improved drugs and therapeutic methods for treating cardiovascular disorders.

Also, current therapies for treating bacterial infections are insufficient because many prominent, infection-causing bacterial strains have developed resistance to current antibiotics. Antibiotic resistance can result in severe adverse outcomes, such as increased mortality, morbidity, and medical care costs for patients suffering from common infections. Infections due to organisms such as methicillin-resistant Staphylococcus aureus (MRSA) occur with increasing frequency in hospitals and are becoming more difficult to treat with conventional antibiotics. For example, a recently discovered strain of Staphylococcus aureus was resistant to treatment with vancomycin, a drug generally regarded as a last line of defense against certain infections. Thus, infection by antibiotic-resistant organisms is a significant health threat for which new methods and compositions for treatment are needed.

Another important medical therapy is blood transfusions. Blood transfusions are a ubiquitious part of healthcare delivery. In the United States (US), someone needs blood about every two seconds and according to the 2009 National Blood Collection and Utilization Survey Report (NBCUS), a total of 15 million units of blood were transfused. Currently, blood products can be stored only for short periods of time. Thus, one unmet medical need is for compositions and methods capable of extending the storage life of blood products.

Accordingly, there is need for new therapeutic methods and compositions for extending the storage life of blood products, enhancing the benefits of blood transfusions, and treating disorders such as cancer, cardiovascular disorders, ischemic conditions, and bacterial infections. The present invention addresses these needs and provides other related advantages.

### SUMMARY

The invention provides methods, compositions, and medical kits comprising a nitrite-reductase promoter, such as an allosteric modulator of hemoglobin, for use in treating medical disorders and preservation of blood products. For example, in certain aspects, the invention provides methods, compositions, and medical kits comprising an inorganic nitrite salt and a nitrite-reductase promoter, such as an allosteric modulator of hemoglobin, for use in treating medical disorders, such as cancer, cardiovascular disorders, ischemic conditions, hemolytic conditions, and bacterial infections. In other aspects, the invention provides agents (e.g., allosteric modulator of hemoglobin) for treating a patient suffering from reduced blood volume (e.g., a patient suffering from hemorrhagic shock), performing a blood transfusion to a patient, treating a patient suffering from anemia, and preserving an isolated blood product. Various aspects and embodiments of the invention are described in further detail below.

One aspect of the invention provides a method of treating or preventing a disorder selected from the group consisting of cancer, a cardiovascular disorder, an ischemic condition, a hemolytic condition, or a bacterial infection. The method comprises administering to a patient in need thereof a therapeutically effective amount of (i) an inorganic nitrite salt, and (ii) an allosteric modulator of hemoglobin that promotes nitrite reductase activity. In certain embodiments, the allosteric modulator of hemoglobin is a compound embraced by Formula I or Formula II, wherein Formula I is represented by:

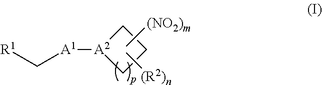

or a pharmaceutically acceptable salt or solvate thereof, wherein the variables are as defined in the detailed description, and Formula II is represented by:

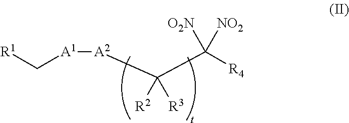

or a pharmaceutically acceptable salt or solvate thereof, wherein the variables are as defined in the detailed description.

Another aspect of the invention provides a method of increasing the amount of nitric oxide produced by hemoglobin in a patient. The method comprises administering to a patient in need thereof a therapeutically effective amount of (i) an inorganic nitrite salt, and (ii) an allosteric modulator of hemoglobin that promotes nitrite reductase activity.

Another aspect of the invention provides a pharmaceutical composition comprising (i) an inorganic nitrite salt, and (ii) an allosteric modulator of hemoglobin that promotes nitrite reductase activity. Still another aspect of the invention provides a kit for treating a medical disorder. The kit comprises (i) an inorganic nitrite salt, (ii) an allosteric modulator of hemoglobin, and (iii) instructions for using the kit to treat a medical disorder.

Another aspect of the invention provides a method of treating a patient suffering from reduced blood volume. The method comprises administering to a patient in need thereof a blood product by injection and a therapeutic agent selected from the group consisting of an organonitro compound of Formula I, organonitro compound of Formula II, hemoglobin conjugate of Formula III, hemoglobin conjugate of Formula IV, and an erythrocyte cell that has been exposed to an organonitro compound of Formula I or II; wherein Formula I is represented by:

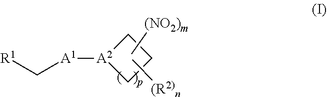

or a pharmaceutically acceptable salt or solvate thereof, wherein the variables are as defined in the detailed description, Formula II is represented by:

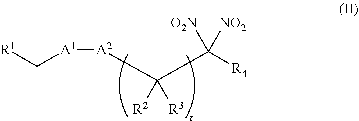

or a pharmaceutically acceptable salt or solvate thereof, wherein, the variables are as defined in the detailed description, Formula III is represented by:

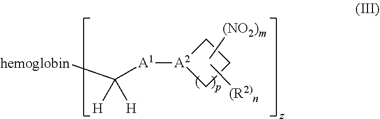

or a pharmaceutically acceptable salt or solvate thereof, wherein the variables are as defined in the detailed description, and Formula IV is represented by:

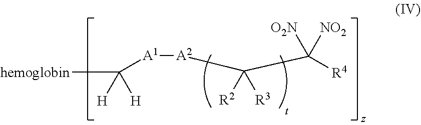

or a pharmaceutically acceptable salt or solvate thereof, wherein the variables are as defined in the detailed description.

Another aspect of the invention provides a method of performing a blood transfusion to a patient. The method comprises administering to a patient in need thereof a blood product by injection and a therapeutic agent selected from the group consisting of an organonitro compound of Formula I, organonitro compound of Formula II, hemoglobin conjugate of Formula III, hemoglobin conjugate of Formula IV, and an erythrocyte cell that has been exposed to an organonitro compound of Formula I or II, wherein Formula I is represented by:

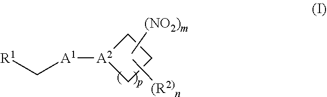

or a pharmaceutically acceptable salt or solvate thereof, wherein the variables are as defined in the detailed description, Formula II is represented by:

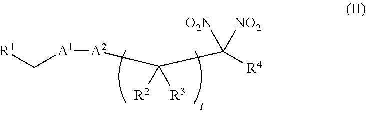

or a pharmaceutically acceptable salt or solvate thereof: wherein the variables are as defined in the detailed description, Formula III is represented by:

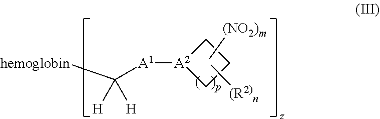

or a pharmaceutically acceptable salt or solvate thereof, wherein the variables are as defined in the detailed description, and Formula IV is represented by:

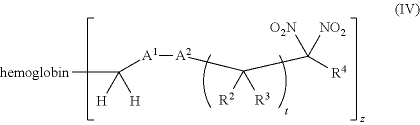

or a pharmaceutically acceptable salt or solvate thereof, wherein the variables are as defined in the detailed description.

Another aspect of the invention provides a method of treating a patient suffering from anemia. The method comprises administering to a patient in need thereof a therapeutic agent selected from the group consisting of an organonitro compound of Formula I, organonitro compound of Formula II, hemoglobin conjugate of Formula III, hemoglobin conjugate of Formula IV, and an erythrocyte cell that has been exposed to an organonitro compound of Formula I or II; wherein Formula I is represented by:

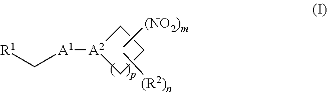

or a pharmaceutically acceptable salt or solvate thereof, wherein the variables are as defined in the detailed description, Formula II is represented by:

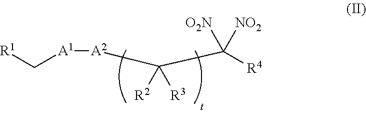

or a pharmaceutically acceptable salt or solvate thereof, wherein the variables are as defined in

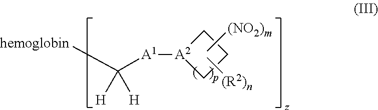

or a pharmaceutically acceptable salt or solvate thereof, wherein the variables are as defined in the detailed description, and Formula IV is represented by:

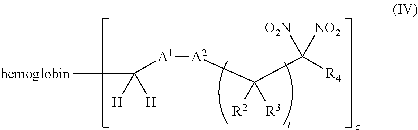

or a pharmaceutically acceptable salt or solvate thereof, wherein the variables are as defined in the detailed description.

Another aspect of the invention provides a method of preserving an isolated blood product. The method comprises exposing the isolated blood product to an agent selected from the group consisting of an organonitro compound of Formula I, organonitro compound of Formula II, hemoglobin conjugate of Formula III, hemoglobin conjugate of Formula IV, and an erythrocyte cell that has been exposed to an organonitro compound of Formula I or II, wherein Formula I is represented by:

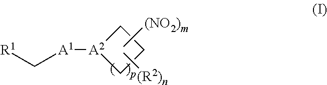

or a pharmaceutically acceptable salt or solvate thereof, wherein the variables are as defined in the detailed description, Formula II is represented by:

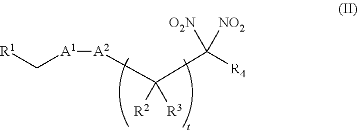

or a pharmaceutically acceptable salt or solvate thereof: wherein the variables are as defined in

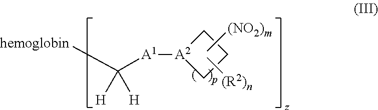

or a pharmaceutically acceptable salt or solvate thereof, wherein the variables are as defined in the detailed description, and Formula IV is represented by:

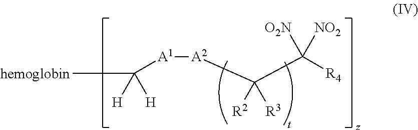

or a pharmaceutically acceptable salt or solvate thereof, wherein the variables are as defined in the detailed description.

Another aspect of the invention provides an isolated blood product composition. The composition comprises (i) a blood product, and (ii) an agent selected from the group consisting of an organonitro compound of Formula I, organonitro compound of Formula II, hemoglobin conjugate of Formula III, hemoglobin conjugate of Formula IV, and an erythrocyte cell that has been exposed to an organonitro compound of Formula I or II; wherein Formula I is represented by:

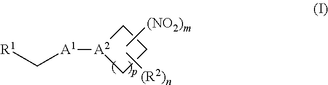

or a pharmaceutically acceptable salt or solvate thereof, wherein the variables are as defined in the detailed description, Formula II is represented by:

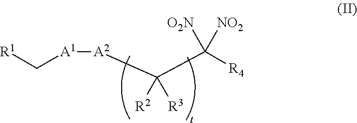

or a pharmaceutically acceptable salt or solvate thereof, wherein the variables are as defined in

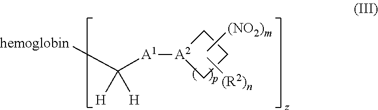

or a pharmaceutically acceptable salt or solvate thereof, wherein the variables are as defined in the detailed description, and Formula IV is represented by:

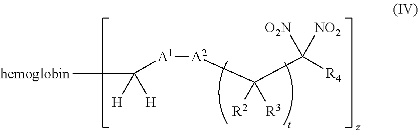

or a pharmaceutically acceptable salt or solvate thereof, wherein the variables are as defined in the detailed description.

Another aspect of the invention provides an isolated hemoglobin conjugate represented by Formula III or IV:

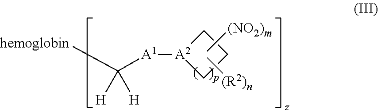

or a pharmaceutically acceptable salt or solvate thereof, wherein the variables are as defined in the detailed description, and Formula IV is represented by:

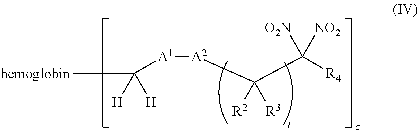

or a pharmaceutically acceptable salt or solvate thereof, wherein the variables are as defined in the detailed description

Another aspect of the invention provides pharmaceutical compositions comprising a pharmaceutically acceptable carrier and a hemoglobin conjugate as defined in the detailed description, such as a hemoglobin conjugate of Formula III or IV.

### BRIEF DESCRIPTION OF THE FIGURES

FIG. 1 is a graph showing the cumulative amount of nitric oxide formed from a blood sample over a thirty-minute time period for multiple experiments (experimental conditions varied include using air atmosphere, N₂ atmosphere, and/or the presence or absence of ABDNAZ), as described in Example 1;

FIG. 2 is a graph showing the cumulative amount of nitric oxide formed from a blood sample over a thirty-minute time period under an air atmosphere or N₂ atmosphere (where do refers to the first experiment, and d1 refers to the second repetition of the experiment), as described in Example 1;

FIG. 3 is a graph showing the cumulative amount of nitric oxide formed from a blood sample mixed with ABDNAZ, where data is shown for a thirty-minute time period under an air atmosphere or N₂ atmosphere (where do refers to the first experiment, and d1 refers to the second repetition of the experiment), as described in Example 1;

FIG. 4 is a graph showing the cumulative amount of nitric oxide formed from a blood sample under an atmosphere of air, where the blood sample is optionally mixed with ABDNAZ (where do refers to the first experiment, and d1 refers to the second repetition of the experiment), as described in Example 1;

FIG. 5 is a graph showing the cumulative amount of nitric oxide formed from a blood sample under an atmosphere of N₂, where the blood sample is optionally mixed with ABDNAZ (where do refers to the first experiment, and d1 refers to the second repetition of the experiment), as described in Example 1;

FIG. 6 is a graph showing the amount of nitric oxide formed in each three-minute period following the start of experiments for multiple experiments (experimental conditions varied include using an air atmosphere, N₂ atmosphere, and/or the presence or absence of ABDNAZ), as described in Example 1;

FIG. 7 is a graph showing the amount of nitric oxide formed in each three-minute period following the start of experiments where the blood sample is under an atmosphere of air or an atmosphere of N₂ as described in Example 1; and

FIG. 8 is a graph showing the amount of nitric oxide formed in each three-minute period following the start of experiments where the blood sample is mixed with ABDNAZ and is under an atmosphere of air or an atmosphere of N₂ as described in Example 1.

FIG. 9 is a bar graph showing percent MetHb for the (i) nitrite, (ii) RRx-001, and (iii) RRx-001+nitrite (RRx-001+N) groups at 60 and 90 minutes post fluid resuscitation (FR), as further described in Example 2. It is noted that the percent MetHb level in normal, healthy individuals is about 1.

FIG. 10 depicts line graphs showing relative changes in arteriolar diameter and blood flow during hemorrhagic shock and resuscitation for all groups tested in Example 2.

FIG. 11 is a line graph showing relative changes in functional capillary density (FCD) during hemorrhagic shock and resuscitation for all groups tested in Example 2. Baseline averages and standard deviations for each of the groups are: Sham, 106±11; Blood, 107±10; Nitrite, 107±12; RRx-001, 112±9; RRx-001±Nitrite, 108±9.

FIG. 12 is a line graph showing calculated vascular resistance (MAP/blood flow) relative to baseline during hemorrhagic shock and resuscitation for all groups tested in Example 2.

FIG. 13 is a bar graph showing the number of apoptotic and necrotic cells at 8 hours following resuscitation for all groups tested in Example 2. Data is presented as the average of fluorescent cells counted in 40 selected visual fields (210×160 μm) for the tissue and the endothelial vessel wall separately. ** P<0.005 for both the number of apoptotic and necrotic in the RRx-001 and RRx-001+nitrite groups compared to blood only.

### DETAILED DESCRIPTION OF THE INVENTION

The invention provides methods, compositions, and medical kits comprising a nitrite-reductase promoter, such as an allosteric modulator of hemoglobin, for use in treating medical disorders and preservation of blood products. For example, in certain aspects, the invention provides methods, compositions, and medical kits comprising an inorganic nitrite salt and a nitrite-reductase promoter, such as an allosteric modulator of hemoglobin, for use in treating medical disorders, such as cancer, cardiovascular disorders, ischemic conditions, hemolytic conditions, and bacterial infections. In other aspects, the invention provides agents (e.g., allosteric modulator of hemoglobin) for treating a patient suffering from reduced blood volume (e.g., a patient suffering from hemorrhagic shock), performing a blood transfusion to a patient, treating a patient suffering from anemia, and preserving an isolated blood product. The practice of the present invention employs, unless otherwise indicated, conventional techniques of organic chemistry, pharmacology, cell biology, and biochemistry. Such techniques are explained in the literature, such as in “Comprehensive Organic Synthesis” (B. M. Trost & I. Fleming, eds., 1991-1992); “Current protocols in molecular biology” (F. M. Ausubel et al., eds., 1987, and periodic updates); and “Current protocols in immunology” (J. E. Coligan et al., eds., 1991), each of which is herein incorporated by reference in its entirety. Various aspects of the invention are set forth below in sections; however, aspects of the invention described in one particular section are not to be limited to any particular section.

### I. Definitions

To facilitate an understanding of the present invention, a number of terms and phrases are defined below.

The terms “a” and “an” as used herein mean “one or more” and include the plural unless the context is inappropriate.

The term “blood product” means (i) whole blood, or (ii) component(s) isolated from whole.

The term “alkyl” as used herein refers to a saturated straight or branched hydrocarbon, such as a straight or branched group of 1-12, 1-10, or 1-6 carbon atoms, referred to herein as C₁-C₁₂alkyl, C₁-C₁₀alkyl, and C₁-C₆alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “haloalkyl” refers to an alkyl group that is substituted with at least one halogen. For example, —CH₂F, —CHF₂, —CF₃, —CH₂CF₃, —CF₂CF₃, and the like.

The term “aralkyl” refers to an alkyl group substituted with an aryl group.

The term “heteroaralkyl” refers to an alkyl group substituted with a heteroaryl group.

The term “aryl” is art-recognized and refers to a carbocyclic aromatic group. Representative aryl groups include phenyl, naphthyl, anthracenyl, and the like. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “aryl” also includes polycyclic ring systems having two or more carbocyclic rings in which two or more carbons are common to two adjoining rings (the rings are “fused rings”) wherein at least one of the rings is aromatic and, e.g., the other ring(s) may be cycloalkyls, cycloalkenyls, cycloalkynyls, and/or aryls. In certain embodiments, the aryl group is not substituted, i.e., it is unsubstituted.

The “heteroaryl” is art-recognized and refers to aromatic groups that include at least one ring heteroatom. In certain instances, a heteroaryl group contains 1, 2, 3, or 4 ring heteroatoms. Representative examples of heteroaryl groups includes pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic ring systems having two or more rings in which two or more carbons are common to two adjoining rings (the rings are “fused rings”) wherein at least one of the rings is heteroaromatic and, e.g., the other ring(s) may be cycloalkyls, cycloalkenyls, cycloalkynyls, and/or aryls. In certain embodiments, the heteroaryl is a bicyclic aromatic ring in which both ring of the bicyclic system are heteroaromatic. In certain embodiments, the heteroaryl group is not substituted, i.e., it is unsubstituted.

The terms ortho, meta and para are art-recognized and refer to 1,2-, 1,3- and 1,4-disubstituted benzenes, respectively. For example, the names 1,2-dimethylbenzene and ortho-dimethylbenzene are synonymous.

As used herein, the term “heterocyclic” represents, for example, an aromatic or nonaromatic ring containing one or more heteroatoms. The heteroatoms can be the same or different from each other. Examples of heteroatoms include, but are not limited to nitrogen, oxygen and sulfur. Aromatic and nonaromatic heterocyclic rings are well-known in the art. Some nonlimiting examples of aromatic heterocyclic rings include pyridine, pyrimidine, indole, purine, quinoline and isoquinoline. Nonlimiting examples of nonaromatic heterocyclic compounds include piperidine, piperazine, morpholine, pyrrolidine and pyrazolidine. Examples of oxygen containing heterocyclic rings include, but not limited to furan, oxirane, 2H-pyran, 4H-pyran, 2H-chromene, and benzofuran. Examples of sulfur-containing heterocyclic rings include, but are not limited to, thiophene, benzothiophene, and parathiazine. Examples of nitrogen containing rings include, but not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine. Examples of heterocyclic rings containing two different heteroatoms include, but are not limited to, phenothiazine, morpholine, parathiazine, oxazine, oxazole, thiazine, and thiazole. The heterocyclic ring is optionally further substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like.

The terms “amine” and “amino” are art-recognized and refer to both unsubstituted and substituted amines, e.g., a moiety represented by the general formula —N(R⁵⁰)(R⁵¹), wherein R⁵⁰ and R⁵¹ each independently represent hydrogen, alkyl, cycloalkyl, heterocyclyl, alkenyl, aryl, aralkyl, or —(CH₂)ₘ—R⁶¹; or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In embodiments, R⁵⁰ and R⁵¹ each independently represent hydrogen, alkyl, alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O— alkenyl, —O-alkynyl, —O—(CH₂)ₘ—R₆₁, where m and R₆₁ are described above.

The terms “ABDNAZ” and “RRx-001” are used interchangeably and refer to the compound having the following structure:

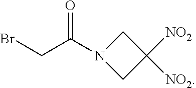 [SMILES: O=C(CBr)N1CC([N+](=O)[O-])([N+](=O)[O-])C1]

Certain compounds contained in compositions of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

As used herein, the terms “subject” and “patient” refer to organisms to be treated by the methods of the present invention. Such organisms are preferably mammals (e.g., murines, simians, equines, bovines, porcines, canines, felines, and the like), and more preferably humans. The term “non-anemic patient” refers to a patient that does not suffer from anemia.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants. (See e.g., Martin, Remington's Pharmaceutical Sciences, 15th Ed., Mack Publ. Co., Easton, PA [1975]).

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “isolated” refers to material that is removed from its original environment (e.g., the natural environment if it is naturally occurring).

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### II. Combination Therapy of Inorganic Nitrite Salt and Nitrite-Reductase Promoter

The invention provides combination therapy using an inorganic nitrite salt in combination with a nitrite-reductase promoter. Exemplary inorganic nitrite salts and exemplary nitrite-reductase promoters for use in the combination therapy methods, pharmaceutical compositions, and medical kits are described below. In addition, because the combination therapy may optionally comprise administration of one or more additional therapeutic agents for treatment of the designated medical disorder, exemplary additional therapeutic agents for treating exemplary medical disorders are described below.

### A. Inorganic Nitrite Salts

The inorganic nitrite salt may be an alkali metal nitrite salt, an alkaline earth metal nitrite salt, or ammonium nitrite salt. Exemplary alkali metal nitrite salts include sodium nitrite, postassium nitrite, lithium nitrite, cesium nitrite, and rubidium nitrite. Exemplary alkaline earth metal nitrite salts include magnesium nitrite, calcium nitrite, barium nitrite, and strontium nitrite. Additional exemplary metal-based inorganic nitrite salts include silver (I) nitrite (AgNO₂), cobalt(II) nitrite (Co(NO₂)₂), and zinc nitrite (Zn(NO₂)₂). The alkali metal nitrite salt, alkaline earth metal nitrite salt, or ammonium nitrite salt may be in the form of solvate, such as a hydrate (e.g., a mono-hydrate or dehydrate). Alternatively, the alkali metal nitrite salt, alkaline earth metal nitrite salt, or ammonium nitrite salt may be anhydrous.

Exemplary ammonium nitrite salts include compounds embraced by the formula NO₂—N(R′)₄, wherein R′ represents independently for each occurrence hydrogen, optionally substituted alkyl, optionally substituted heteroalkyl, optionally substituted cycloalkyl, optionally substituted heterocycloalkyl, optionally substituted aryl, optionally substituted aralkyl, optionally substituted heteroaryl, or optionally substituted heteroaralkyl. In certain other embodiments, the ammonium nitrite salt is arginine nitrite, ammonium nitrite (NH₄NO₂), or tetramethylammonium nitrite.

### B. Nitrite-Reductase Promoters

The nitrite reductase promoter enhances conversion of nitrite to nitric oxide in vivo. One exemplary class of nitrite-reductase promoters is an allosteric modulator of hemoglobin, such as compounds that bind to the beta-cysteine-93 residue of hemoglobin to enhance the nitrite-reductase activity of hemoglobin. Another exemplary class of nitrite-reductase promoters is an agent that modulates the oxygen binding affinity of hemoglobin and/or erythrocyte cells, such as an agent that increases oxygen binding affinity of hemoglobin and/or erythrocyte cells. Co-administration of a nitrite reductase promoter with an inorganic nitrite salt results in increased levels of nitric oxide in vivo. One benefit of the combination therapy is that the nitrite reductase promoter allows for generation of beneficial levels of nitric oxide in vivo, while minimizing the amount of inorganic nitrite salt that must be administered to the patient.

Exemplary allosteric modulators of hemoglobin contemplated for use in the methods, compositions, and kits include nitrosating agents such as S-nitroso-N-acetylcysteine, S-nitrosocysteinylglycine, S-nitrosocysteine, S-nitrosohomocysteine, metal nitrosyl complexes, S-nitro compounds, S-nitroso compounds, thionitrites, diazeniumdiolates, and other related nitrosating agents as described in Feelisch, M. and Stamler, J. S., “Donors of Nitrogen Oxides” chapter 7, pp. 71-115 in Methods in Nitric Oxide Research (Freelisch, M. and Stamler, J. S., eds.) John Wiley and Sons, Ltd., Chichester, U.K. (1996), the contents of which are hereby incorporated by reference in their entirety. A nitrosating agent can be chosen for minimal oxidation of the heme iron of hemoglobin, and maximum activity in nitosylating thiol groups such as found on cysteine. Other exemplary allosteric modulators of hemoglobin contemplated for use in the methods, compositions, and kits include 4-pyridylmethyl chloride, an alkoxyalkylchloride, dimethoxymethane, N-(hydroxymethyl)acetamide, triphenylmethyl chloride, acetyl chloride, 2-chloroacetic acid, acetic anhydride, a haloacetamide (such as, iodoacetamide, bromoacetamide, chloroacetamide, or fluoroacetamide), a haloacetate (such as iodoacetate, bromoacetate, chloroacetate, or fluoroacetate), benzyl chloride, benzoyl chloride, di-tert-butyl dicarbonate, p-hydroxyphenacyl bromide, p-acetoxybenzyl chloride, p-methoxybenzyl chloride, 2,4-dinitrophenyl fluoride, tetrahydropyran, acetamidohydroxymethane, acetone, bis-carboethoxyethene, 2,2,2-trichloroethoxycarbonyl chloride, tert-butoxycarbonyl chloride, an alkyl isocyanate, and an alkoxyalkyl isocyanate. In certain other embodiments, the allosteric modulator of hemoglobin is an optionally substituted alkyl-R*, optionally substituted aralkyl-R*, or optionally substituted heteroaralkyl-R*, wherein R* is a leaving group, such as halogen, an alkyl sulfonate, arylsulfonate, alkyl acetate, or haloalkyl acetate. In certain other embodiments, the allosteric modulator of hemoglobin is an optionally substituted alkyl-NCO, optionally substituted aryl-NCO, optionally substituted aralkyl-NCO, optionally substituted heterocycyl-NCO, optionally substituted heteroaryl-NCO, or optionally substituted heteroaralkyl-NCO. In certain other embodiments, the allosteric modulator of hemoglobin is an optionally substituted alkyl-C(O)X, optionally substituted aryl-C(O)X, optionally substituted aralkyl-C(O)X, optionally substituted heterocycyl-C(O)X, optionally substituted heteroaryl-C(O)X, or optionally substituted heteroaralkyl-C(O)X, where X is a leaving group, such as halogen or —OC(O)alkyl.

In certain embodiments, the sulfhydryl of the β93-cysteine on hemoglobin may be alkylated with an allosteric modulator of hemoglobin that is a derivatized dextran. For example, in certain embodiments, the dextran may be derivatized to contain a free amino group (e.g., using cyanogen bromide and diaminoethane), and the free amino group may be acylated with an alkylating moiety (e.g., bromoacetyl bromide) that can alkylate the sulfhydryl of the β93-cysteine.

In certain other embodiments, the allosteric modulator of hemoglobin is a polyalkylene glycol. Polyalkylene glycols containing a reactive group are contemplated to react with the β93-cysteine residue of hemoglobin to modulate hemoglobin activity. In certain embodiments, the polyalkylene glycol contains a maleimide group, such as (polyethylene glycol)-maleimide. In certain other embodiments, the polyalkylene glycol contains a N-hydroxysuccinimide group. The polyethylene glycol may have a weight average molecular weight of about 200 g/mol to about 100,000 g/mol, about 200 g/mol to about 20,000 g/mol, about 200 g/mol to about 1,000 g/mol, or about 1,000 g/mol to about 10,000 g/mol.

In certain other embodiments, the allosteric modulator of hemoglobin is an organonitro compound embraced by Formula I:

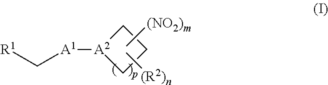

or a pharmaceutically acceptable salt or solvate thereof, wherein:
A¹ is —C(O)— or —(C(R³)₂)ₓC(O)(C(R³)₂)ₓ—;
A² is N or —C(R⁴)—;
R¹ is halogen, —OS(O)₂R⁵, or —OC(O)CF₃;
R² is C₁-C₆alkyl;
R³ and R⁴ each represent independently for each occurrence hydrogen or C₁-C₅alkyl;
R⁵ is C₁-C₆alkyl, C₁-C₆haloalkyl, aryl, or aralkyl;
m and p are independently 1, 2, or 3; and n and x each represent independently for each occurrence 0, 1, 2, or 3.

In certain embodiments, the allosteric modulator of hemoglobin is an organonitro compound embraced by Formula I as defined by particular definitions for variables in Formula I, such as where A¹ is —C(O)—. In certain other embodiments, A¹ is —(C(R³)₂)ₓC(O)(C(R³)₂)ₓ—. In certain other embodiments, A¹ is —C(O)(C(R³)₂)ₓ—.

In certain embodiments, A² is N. In certain other embodiments, A² is —C(R⁴)—.

In certain embodiments, R¹ is halogen, —OS(O)₂R5, or —OC(O)CF₃. In certain other embodiments, R¹ is halogen. In certain other embodiments, R¹ is —OS(O)₂R⁵. In certain other embodiments, R¹ is —OC(O)CF₃. In certain other embodiments, R¹ is chloro, bromo, —OS(O)₂-(para-methylphenyl), —OS(O)₂CH₃, —OS(O)₂CF₃, or —OC(O)CF₃. In certain embodiments, R¹ is bromo.

In certain embodiments, m is 2. In certain other embodiments, m is 1.

In certain embodiments, n is 0. In certain other embodiments, n is 1. In certain other embodiments, n is 2.

In certain embodiments, p is 1. In certain other embodiments, p is 2. In certain other embodiments, p is 3.

The description above describes multiple embodiments relating to compounds of Formula I. The patent application specifically contemplates all combinations of the embodiments. For example, the invention contemplates a compound of Formula I wherein A¹ is —C(O)—, A² is N, R¹ is halogen, and n is 0.

In certain embodiments, the compound is a compound of Formula I-A:

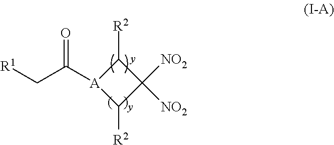

or a pharmaceutically acceptable salt or solvate thereof, wherein:
A is N or C(H);
R¹ is chloro, bromo, —OS(O)₂—(C₁-C₆alkyl), —OS(O)₂—(C₁-C₆haloalkyl), —OS(O)₂-(para-methylphenyl), or —OC(O)CF₃;
R² represents independently for each occurrence hydrogen or methyl; and
y represents independently for each occurrence 1 or 2.

In certain embodiments, the allosteric modulator of hemoglobin is an organonitro compound embraced by Formula I-A as defined by particular definitions for variables in Formula I-A, such as where A is N. In certain other embodiments, A is C(H).

In certain embodiments, R¹ is chloro or bromo. In certain embodiments, R¹ is chloro. In certain other embodiments, R¹ is bromo. In certain embodiments, R¹ is —OS(O)₂—(C₁-C₆alkyl), —OS(O)₂—(C₁-C₆haloalkyl), or —OS(O)₂-(para-methylphenyl). In certain other embodiments, R¹ is —OS(O)₂CH₃, —OS(O)₂CF₃, or —OS(O)₂-(para-methylphenyl). In certain other embodiments, R¹ is —OC(O)CF₃.

In certain embodiments, R² is hydrogen or methyl. In certain embodiments, R2 is hydrogen.

In certain embodiments, y is 1. In certain embodiments, one occurrence of y is 1, and the other occurrence of y is 2. In certain other embodiments, y is 2.

The description above describes multiple embodiments relating to compounds of Formula I-A. The patent application specifically contemplates all combinations of the embodiments. For example, the invention contemplates a compound of Formula I-A wherein A is N, R¹ is chloro or bromo, and R² is hydrogen.

In certain embodiments, the compound is

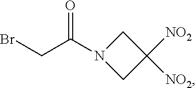 [SMILES: O=C(CBr)N1CC([N+](=O)[O-])([N+](=O)[O-])C1]

or a pharmaceutically acceptable salt or solvate thereof. In certain other embodiments, the compound is

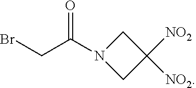 [SMILES: O=C(CBr)N1CC([N+](=O)[O-])([N+](=O)[O-])C1]

In certain other embodiments, the allosteric modulator of hemoglobin is an organonitro compound embraced by Formula II:

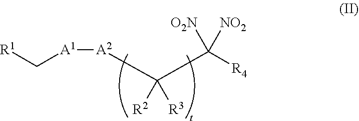

or a pharmaceutically acceptable salt or solvate thereof: wherein:
A¹ is —C(O)— or —(C(R⁵)₂)ₓC(O)(C(R⁵)₂)ₓ—;
A² is —N(R⁵)— or —C(R²)(R³)—.
R¹ is halogen, —OS(O)₂R⁶, or —OC(O)CF₃;
R² and R³ each represent independently for each occurrence hydrogen or C₁-C₆alkyl; or R² and R³ are taken together with the carbon atom to which they are attached to form a 3-6 membered, saturated carbocyclic ring;
R⁴ is hydrogen or C₁-C₆alkyl;
R⁵ represents independently for each occurrence hydrogen or C₁-C₆alkyl;
R⁶ is C₁-C₆alkyl, C₁-C₆haloalkyl, aryl, or aralkyl;
t is an integer in the range from 1 to 12; and
x represents independently for each occurrence 0, 1, 2, or 3.

In certain embodiments, the allosteric modulator of hemoglobin is an organonitro compound embraced by Formula II as defined by particular definitions for variables in Formula II, such as where A¹ is —C(O)—. In certain other embodiments, A¹ is —(C(R⁵)₂)ₓC(O)(C(R⁵)₂)ₓ—. In certain other embodiments, A¹ is —C(O)(C(R⁵)₂)ₓ—.

In certain embodiments, A² is —N(R⁵)—. In certain other embodiments, A² is —C(R²)(R³)—.

In certain embodiments, R¹ is halogen. In certain other embodiments, R¹ is —OS(O)₂R⁶. In certain other embodiments, R¹ is —OC(O)CF₃. In certain other embodiments, R¹ is chloro, bromo, —OS(O)₂-(para-methylphenyl), —OS(O)₂CH₃, —OS(O)₂CF₃, or —OC(O)CF₃. In certain embodiments, R¹ is bromo.

In certain embodiments, R² and R³ each represent independently for each occurrence hydrogen or C₁-C₆alkyl. In certain other embodiments, R² and R³ each represent independently for each occurrence hydrogen, methyl, ethyl, or propyl. In certain other embodiments, R² and R³ each represent independently for each occurrence hydrogen or methyl. In certain embodiments, R² and R³ are hydrogen.

In certain embodiments, R⁴ is hydrogen, methyl, ethyl, propyl, butyl, or pentyl. In certain other embodiments, R⁴ is methyl, ethyl or propyl. In certain other embodiments, R⁴ is methyl.

In certain embodiments, R⁵ is hydrogen or methyl. In certain other embodiments, R⁵ is hydrogen.

In certain embodiments, R⁶ is C₁-C₆alkyl or C₁-C₆haloalkyl. In certain other embodiments, R⁶ is methyl, ethyl, or trifluoromethyl. In certain other embodiments, R⁶ is aryl, such as phenyl.

In certain embodiments, t is 1, 2, 3, 4, 5 or 6. In certain other embodiments, t is 1, 2, or 3. In certain other embodiments, t is 1. In certain embodiments, x is 1 or 2.

The description above describes multiple embodiments relating to compounds of Formula II. The patent application specifically contemplates all combinations of the embodiments. For example, the invention contemplates a compound of Formula II wherein A¹ is —C(O)—, A² is —N(R⁵)—, and R² and R³ are hydrogen.

In certain embodiments, the compound is a compound of Formula II-A:

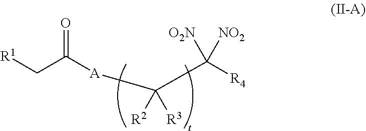

or a pharmaceutically acceptable salt or solvate thereof: wherein:
A is —N(R⁵)— or —C(R²)(R³)—.
R¹ is chloro, bromo, —OS(O)₂—(C₁-C₆alkyl), —OS(O)₂—(C₁-C₆haloalkyl), —OS(O)₂-(para-methylphenyl), or —OC(O)CF₃;
R², R³, and R⁵ each represent independently for each occurrence hydrogen or methyl;
R⁴ is hydrogen or C₁-C₆alkyl; and
t is 1, 2, or 3.

In certain embodiments, the allosteric modulator of hemoglobin is an organonitro compound embraced by Formula II-A as defined by particular definitions for variables in Formula II-A, such as where A is —N(R⁵)—. In certain other embodiments, A is —N(CH₃)—. In certain other embodiments, A is —C(R²)(R³)—. In certain other embodiments, A is —CH₂—.

In certain embodiments, R¹ is chloro. In certain other embodiments, R¹ is bromo. In certain embodiments, R¹ is —OS(O)₂—(C₁-C₆alkyl), —OS(O)₂—(C₁-C₆haloalkyl), or —OS(O)₂-(para-methylphenyl). In certain other embodiments, R¹ is —OS(O)₂CH₃, —OS(O)₂CF₃, or —OS(O)₂-(para-methylphenyl). In certain other embodiments, R¹ is —OC(O)CF₃.

In certain embodiments, R² and R³ are hydrogen.

In certain embodiments, R⁴ is hydrogen, methyl, ethyl, propyl, butyl, or pentyl. In certain other embodiments, R⁴ is methyl, ethyl or propyl. In certain other embodiments, R⁴ is methyl.

In certain embodiments, R⁵ is hydrogen or methyl. In certain other embodiments, R⁵ is hydrogen.

The description above describes multiple embodiments relating to compounds of Formula II-A. The patent application specifically contemplates all combinations of the embodiments. For example, the invention contemplates a compound of Formula II-A wherein A is —N(R⁵)—, and R² and R³ are hydrogen.

In certain other embodiments, the allosteric modulator of hemoglobin is one of the compounds listed in Tables 1, 2, or 3 below or a pharmaceutically acceptable salt or solvate thereof.

TABLE 1

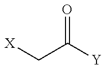

| Com- | | |
|---|---|---|
| pound | | |
| No. | X | Y |
| I-1 | Br | 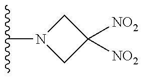 [SMILES: *N1CC([N+](=O)[O-])([N+](=O)[O-])C1] |
| I-2 | Br | 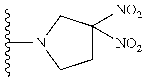 [SMILES: *N1CCC([N+](=O)[O-])([N+](=O)[O-])C1] |
| I-3 | Br | 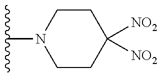 [SMILES: *N1CCC([N+](=O)[O-])([N+](=O)[O-])CC1] |
| I-4 | Br | 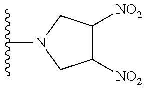 [SMILES: *N1CC([N+](=O)[O-])C([N+](=O)[O-])C1] |
| I-5 | Br | 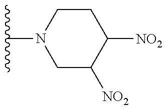 [SMILES: *N1CCC([N+](=O)[O-])C([N+](=O)[O-])C1] |
| I-6 | Br | 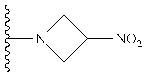 [SMILES: *N1CC([N+](=O)[O-])C1] |
| I-7 | Br | 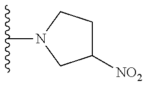 [SMILES: *N1CCC([N+](=O)[O-])C1] |
| I-8 | Br | 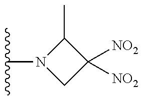 [SMILES: *N1CC([N+](=O)[O-])([N+](=O)[O-])C1C] |
| I-9 | Br | 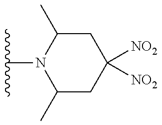 [SMILES: *N1C(C)CC([N+](=O)[O-])([N+](=O)[O-])CC1C] |
| I-10 | Br | 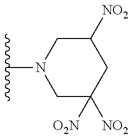 [SMILES: *N1CC([N+](=O)[O-])CC([N+](=O)[O-])([N+](=O)[O-])C1] |
| I-11 | Br | 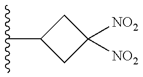 [SMILES: *C1CC([N+](=O)[O-])([N+](=O)[O-])C1] |
| I-12 | Br | 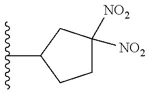 [SMILES: *C1CCC([N+](=O)[O-])([N+](=O)[O-])C1] |
| I-13 | Br | 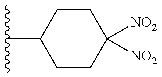 [SMILES: *C1CCC([N+](=O)[O-])([N+](=O)[O-])CC1] |
| I-14 | Br | 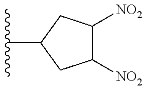 [SMILES: *C1CC([N+](=O)[O-])C([N+](=O)[O-])C1] |
| I-15 | Br | 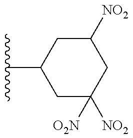 [SMILES: *C1CC([N+](=O)[O-])CC([N+](=O)[O-])([N+](=O)[O-])C1] |
| I-16 | Br | 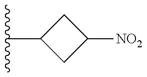 [SMILES: *C1CC([N+](=O)[O-])C1] |
| I-17 | Br | 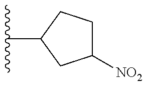 [SMILES: *C1CCC([N+](=O)[O-])C1] |
| I-18 | Br | 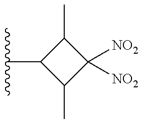 [SMILES: *C1C(C)C([N+](=O)[O-])([N+](=O)[O-])C1C] |
| I-19 | Cl | 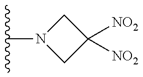 [SMILES: *N1CC([N+](=O)[O-])([N+](=O)[O-])C1] |
| I-20 | Cl | 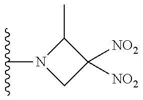 [SMILES: *N1CC([N+](=O)[O-])([N+](=O)[O-])C1C] |
| I-21 | Cl | 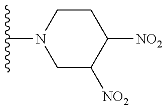 [SMILES: *N1CCC([N+](=O)[O-])C([N+](=O)[O-])C1] |
| I-22 | Cl | 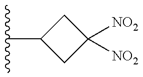 [SMILES: *C1CC([N+](=O)[O-])([N+](=O)[O-])C1] |
| I-23 | I | 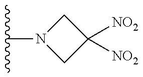 [SMILES: *N1CC([N+](=O)[O-])([N+](=O)[O-])C1] |
| I-24 | I | 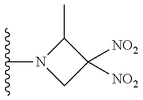 [SMILES: *N1CC([N+](=O)[O-])([N+](=O)[O-])C1C] |
| I-25 | I | 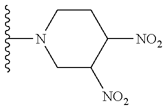 [SMILES: *N1CCC([N+](=O)[O-])C([N+](=O)[O-])C1] |
| I-26 | I | 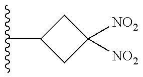 [SMILES: *C1CC([N+](=O)[O-])([N+](=O)[O-])C1] |
| I-27 | —OS(O)₂CH₃ | 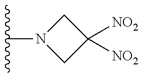 [SMILES: *N1CC([N+](=O)[O-])([N+](=O)[O-])C1] |
| I-28 | —OS(O)₂CH₃ | 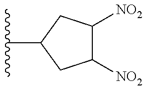 [SMILES: *C1CC([N+](=O)[O-])C([N+](=O)[O-])C1] |
| I-29 | —OS(O)₂CF₃ | 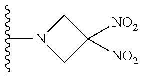 [SMILES: *N1CC([N+](=O)[O-])([N+](=O)[O-])C1] |
| I-30 | —OS(O)₂CF₃ | 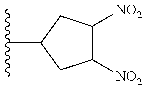 [SMILES: *C1CC([N+](=O)[O-])C([N+](=O)[O-])C1] |
| I-31 | 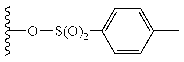 | 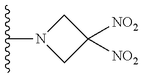 [SMILES: *N1CC([N+](=O)[O-])([N+](=O)[O-])C1] |
| I-32 | 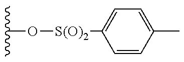 | 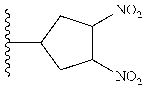 [SMILES: *C1CC([N+](=O)[O-])C([N+](=O)[O-])C1] |
| I-33 | —OC(O)CF₃ | 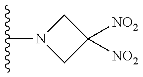 [SMILES: *N1CC([N+](=O)[O-])([N+](=O)[O-])C1] |
| I-34 | —OC(O)CF₃ | 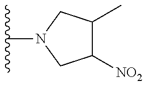 [SMILES: *N1CC(C)C([N+](=O)[O-])C1] |
| I-35 | —OC(O)CF₃ | 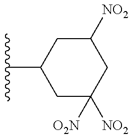 [SMILES: *C1CC([N+](=O)[O-])CC([N+](=O)[O-])([N+](=O)[O-])C1] |

TABLE 2

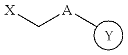

| Compound | | | |
|---|---|---|---|
| No. | X | A | Y |
| II-1 | Br | —CH₂C(O)— | 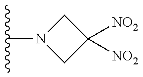 [SMILES: *N1CC([N+](=O)[O-])([N+](=O)[O-])C1] |
| II-2 | Br | —CH₂C(O)— | 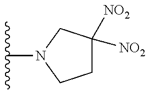 [SMILES: *N1CCC([N+](=O)[O-])([N+](=O)[O-])C1] |
| II-3 | Br | —CH₂C(O)— | 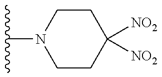 [SMILES: *N1CCC([N+](=O)[O-])([N+](=O)[O-])CC1] |
| II-4 | Br | —CH₂C(O)— | 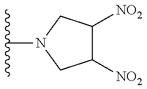 [SMILES: *N1CC([N+](=O)[O-])C([N+](=O)[O-])C1] |
| II-5 | Br | —CH₂C(O)— | 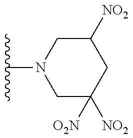 [SMILES: *N1CC([N+](=O)[O-])CC([N+](=O)[O-])([N+](=O)[O-])C1] |
| II-6 | Br | —CH₂C(O)— | 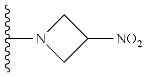 [SMILES: *N1CC([N+](=O)[O-])C1] |
| II-7 | Br | —CH₂C(O)— | 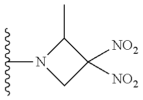 [SMILES: *N1CC([N+](=O)[O-])([N+](=O)[O-])C1C] |
| II-8 | Br | —C(O)CH₂CH₂— | 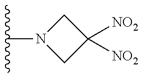 [SMILES: *N1CC([N+](=O)[O-])([N+](=O)[O-])C1] |
| II-9 | Br | —C(O)CH₂CH₂— | 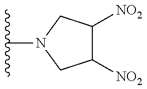 [SMILES: *N1CC([N+](=O)[O-])C([N+](=O)[O-])C1] |
| II-10 | Br | —C(O)CH₂CH₂— | 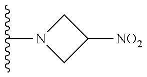 [SMILES: *N1CC([N+](=O)[O-])C1] |
| II-11 | Br | —C(O)CH₂CH₂— | 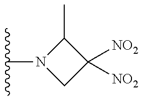 [SMILES: *N1CC([N+](=O)[O-])([N+](=O)[O-])C1C] |
| II-12 | Br | —CH₂— | 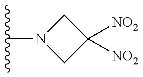 [SMILES: *N1CC([N+](=O)[O-])([N+](=O)[O-])C1] |
| II-13 | Br | —CH₂— | 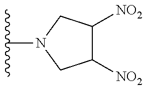 [SMILES: *N1CC([N+](=O)[O-])C([N+](=O)[O-])C1] |
| II-14 | Br | —CH₂— | 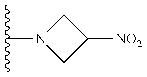 [SMILES: *N1CC([N+](=O)[O-])C1] |
| II-15 | Br | —CH₂— | 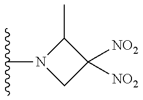 [SMILES: *N1CC([N+](=O)[O-])([N+](=O)[O-])C1C] |
| II-16 | Br | —CH₂C(O)— | 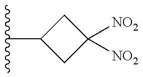 [SMILES: *C1CC([N+](=O)[O-])([N+](=O)[O-])C1] |
| II-17 | Br | —CH₂C(O)— | 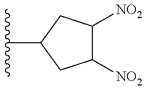 [SMILES: *C1CC([N+](=O)[O-])C([N+](=O)[O-])C1] |
| II-18 | Br | —C(O)CH₂CH₂— | 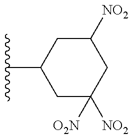 [SMILES: *C1CC([N+](=O)[O-])CC([N+](=O)[O-])([N+](=O)[O-])C1] |
| II-19 | Br | —C(O)CH₂CH₂— | 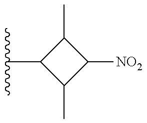 [SMILES: *C1C(C)C([N+](=O)[O-])C1C] |
| II-20 | Br | —CH₂— | 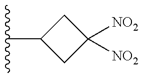 [SMILES: *C1CC([N+](=O)[O-])([N+](=O)[O-])C1] |
| II-21 | Br | —CH₂— | 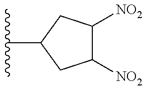 [SMILES: *C1CC([N+](=O)[O-])C([N+](=O)[O-])C1] |
| II-22 | Br | —CH₂— | 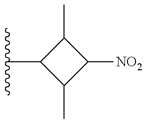 [SMILES: *C1C(C)C([N+](=O)[O-])C1C] |
| II-23 | Cl | —CH₂C(O)— | 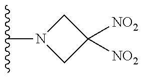 [SMILES: *N1CC([N+](=O)[O-])([N+](=O)[O-])C1] |
| II-24 | Cl | —C(O)CH₂CH₂— | 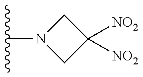 [SMILES: *N1CC([N+](=O)[O-])([N+](=O)[O-])C1] |
| II-25 | Cl | —CH₂— | 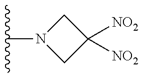 [SMILES: *N1CC([N+](=O)[O-])([N+](=O)[O-])C1] |
| II-26 | Cl | —CH₂C(O)— | 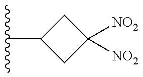 [SMILES: *C1CC([N+](=O)[O-])([N+](=O)[O-])C1] |
| II-27 | Cl | —C(O)CH₂CH₂— | 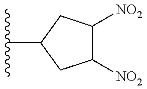 [SMILES: *C1CC([N+](=O)[O-])C([N+](=O)[O-])C1] |
| II-28 | Cl | —CH₂— | 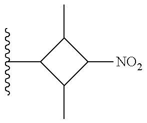 [SMILES: *C1C(C)C([N+](=O)[O-])C1C] |
| II-29 | —OS(O)₂CH₃ | —CH₂C(O)— | 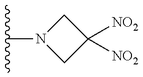 [SMILES: *N1CC([N+](=O)[O-])([N+](=O)[O-])C1] |
| II-30 | —OS(O)₂CH₃ | —CH₂C(O)— | 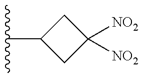 [SMILES: *C1CC([N+](=O)[O-])([N+](=O)[O-])C1] |
| II-31 | —OS(O)₂CH₃ | —C(O)CH₂CH₂— | 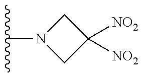 [SMILES: *N1CC([N+](=O)[O-])([N+](=O)[O-])C1] |
| II-32 | —OS(O)₂CH₃ | —CH₂— | 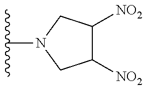 [SMILES: *N1CC([N+](=O)[O-])C([N+](=O)[O-])C1] |
| II-33 | —OC(O)₂CF₃ | —CH₂C(O)− | 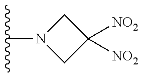 [SMILES: *N1CC([N+](=O)[O-])([N+](=O)[O-])C1] |
| II-34 | —OS(O)₂CF₃ | —CH₂C(O)− | 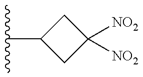 [SMILES: *C1CC([N+](=O)[O-])([N+](=O)[O-])C1] |
| II-35 | —OS(O)₂CF₃ | —C(O)CH₂CH₂— | 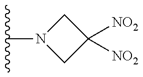 [SMILES: *N1CC([N+](=O)[O-])([N+](=O)[O-])C1] |
| II-36 | —OS(O)₂CF₃ | —CH₂— | 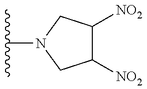 [SMILES: *N1CC([N+](=O)[O-])C([N+](=O)[O-])C1] |
| II-37 | 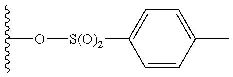 | —CH₂C(O)— | 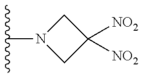 [SMILES: *N1CC([N+](=O)[O-])([N+](=O)[O-])C1] |
| II-38 | 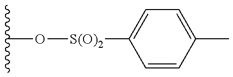 | —CH₂C(O)— | 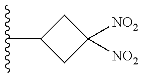 [SMILES: *C1CC([N+](=O)[O-])([N+](=O)[O-])C1] |
| II-39 | 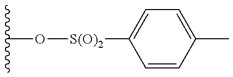 | —C(O)CH₂CH₂— | 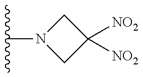 [SMILES: *N1CC([N+](=O)[O-])([N+](=O)[O-])C1] |
| II-40 | 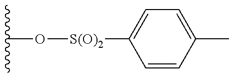 | —CH₂— | 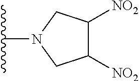 [SMILES: *N1CC([N+](=O)[O-])C([N+](=O)[O-])C1] |
| II-41 | —OC(O)CF₃ | —CH₂C(O)— | 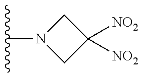 [SMILES: *N1CC([N+](=O)[O-])([N+](=O)[O-])C1] |
| II-42 | —OC(O)CF₃ | —CH₂C(O)— | 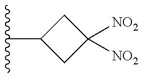 [SMILES: *C1CC([N+](=O)[O-])([N+](=O)[O-])C1] |
| II-43 | —OC(O)CF₃ | —C(O)CH₂CH₂— | 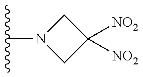 [SMILES: *N1CC([N+](=O)[O-])([N+](=O)[O-])C1] |
| II-44 | —OC(O)CF₃ | —CH₂— | 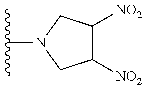 [SMILES: *N1CC([N+](=O)[O-])C([N+](=O)[O-])C1] |

TABLE 3

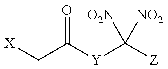

| Compound | | | |
|---|---|---|---|
| No. | X | Y | Z |
| III-1 | Br | —N(H)CH₂— | methyl |
| III-2 | Br | —N(H)CH₂— | ethyl |
| III-3 | Br | —N(H)CH₂— | n-pentyl |
| III-4 | Br | —N(H)CH₂— | hydrogen |
| III-5 | Br | —N(H)CH₂CH₂— | methyl |
| III-6 | Br | —N(H)(CH₂)₄— | methyl |
| III-7 | Br | —N(CH₃)CH₂— | methyl |
| III-8 | Br | —N(CH₃)(CH₂)₃— | methyl |
| III-9 | Br | —N(H)C(CH₃(H)— | methyl |
| III-10 | Br | —N(H)C(CH₃)(H)CH₂— | methyl |
| III-11 | Br | —CH₂— | methyl |
| III-12 | Br | —(CH₂)₂— | methyl |
| III-13 | Br | —CH₂— | ethyl |
| III-14 | Br | —(CH₂)₄— | isopropyl |
| III-15 | Br | —(CH₂)₂— | n-pentyl |
| III-16 | Br | —CH₂— | hydrogen |
| III-17 | Br | —CH₂CH₂C(CH₃)₂— | methyl |
| III-18 | Br | —CH₂C(CH₃)₂CH₂— | methyl |
| III-19 | Cl | —N(H)CH₂— | methyl |
| III-20 | Cl | —N(H)CH₂— | ethyl |
| III-21 | Cl | —N(CH₃)CH₂— | methyl |
| III-22 | Cl | —(CH₂)₂— | methyl |
| III-23 | I | —N(H)CH₂— | methyl |
| III-24 | I | —N(H)CH₂— | ethyl |
| III-25 | I | —N(CH₃)CH₂— | methyl |
| III-26 | I | —(CH₂)₂— | methyl |
| III-27 | —OS(O)₂CH₃ | —N(H)CH₂— | methyl |
| III-28 | —OS(O)₂CH₃ | —N(CH₃)CH₂— | methyl |
| III-29 | —OS(O)₂CF₃ | —N(H)CH₂— | methyl |
| III-30 | —OS(O)₂CF₃ | —N(CH₃)CH₂— | methyl |
| III-31 | 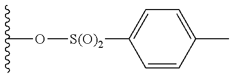 | —N(H)CH₂— | methyl |
| III-32 | 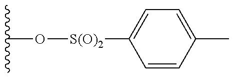 | —N(CH₃)CH₂— | methyl |
| III-33 | —OC(O)CF₃ | —N(H)CH₂— | methyl |
| III-34 | —OC(O)CF₃ | —N(CH₃)CH₂— | methyl |
| III-35 | —OC(O)CF₃ | —(CH₂)₂— | methyl |

Methods for preparing compounds described herein are illustrated in the following synthetic schemes. These schemes are given for the purpose of illustrating the invention, and should not be regarded in any manner as limiting the scope or the spirit of the invention. Starting materials shown in the schemes can be obtained from commercial sources or can be prepared based on procedures described in the literature.

The synthetic route illustrated in Scheme 1 depicts a general method for preparing cyclic geminal di-nitro compounds. In the first step, chloro epoxide A1 is reacted with t-butylamine to provide hydroxy heterocyclic compound B1. Mesylation of the hydroxyl group of heterocyclic compound B1 with methylsulfonyl chloride gives mesylate C1, which upon reacting with NaNO₂ generates cyclic mono-nitro compound D1. Further nitration of compound D1 can be carried out using NaNO₂ in the presence of Na₂S₂O₈ and K₃Fe(CN)₆ to provide geminal di-nitro heterocyclic compound E1. Reacting compound E1 with boron trifluoride etherate and acetyl bromide F provides the desired product G1. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein.

This synthetic procedure illustrated in Scheme 1 and described above is contemplated to be applicable to preparing compounds having various substituents at the R₁, R₂, R₃ and R₄ positions. If a particular epoxide compound embraced by A1 should contain a functional group sensitive to one or more of the synthetic transformations in Scheme 1, then standard protecting group strategies are contemplated to be applied. For further description of protecting group strategies and procedures, see, for example, Greene, T. W.; Wuts, P. G. M. Protective Groups in Organic Synthesis, 2^{nd} ed.; Wiley, New York, 1991.

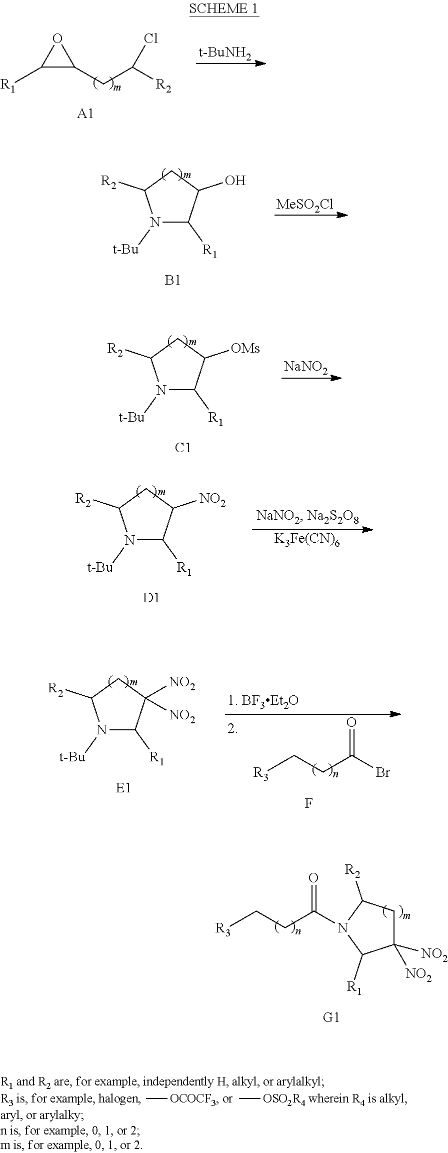

Scheme 2 illustrates a more specific embodiment of the synthetic route shown in Scheme 1 when m is 0. In the first step, epoxide A2 is reacted with t-butylamine to provide hydroxyl azetidine B2. Mesylation of the hydroxyl group of azetidine B2 with methylsulfonyl chloride gives azetidine mesylate C2, which upon reacting with NaNO₂ generates mono-nitro azetidine D2. Further nitration of mono-nitro azetidnine D2 with NaNO₂ in the presence of Na₂S₂O₈ and K₃Fe(CN)₆ furnishes the geminal di-nitro azetidine E2. Reaction of azetidine E2 with boron trifluoride etherate and acetyl bromide compound F provides the desired di-nitro azetidine product G2. This synthetic procedure is contemplated to be applicable to preparing compounds having various substituents at the R₁, R₂, R₃ and R₄ positions. If a particular epoxide compound embraced by A2 should contain a functional group sensitive to one or more of the synthetic transformations in Scheme 2, then standard protecting group strategies are contemplated to be applied. For further description of protecting group strategies and procedures, see, for example, Greene, T. W.; Wuts, P. G. M. Protective Groups in Organic Synthesis, 2^{nd} ed.; Wiley, New York, 1991. Furthermore, mono-nitro compounds can be prepared by treating mono-nitro compound D2 with a Lewis Acid (e.g., boron trifluoride etherate) and acetyl bromide compound F to provide the desired mono-nitro product.

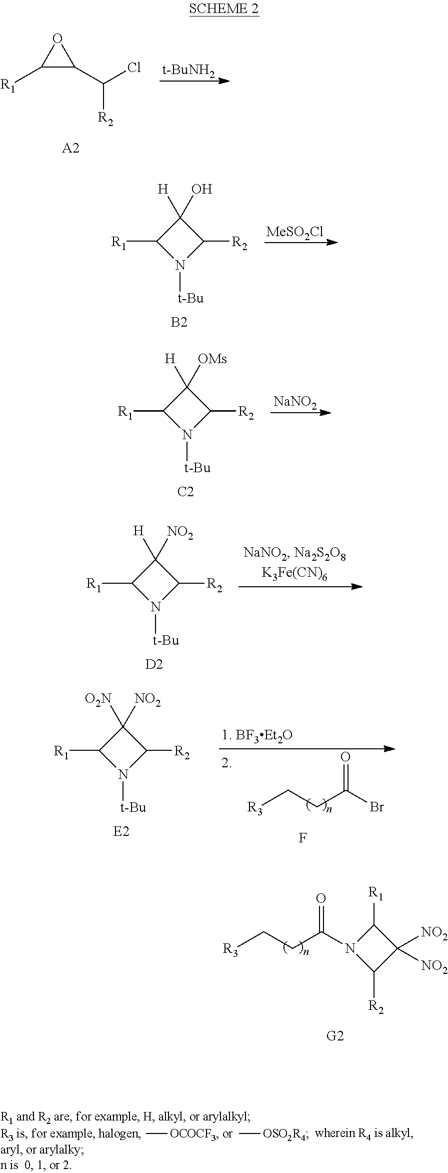

Scheme 3 illustrates another more particular embodiment of the synthetic route shown in Scheme 1 when both R₁ and R₂ are hydrogen and m is 0. In the first step, commercially available epichlorohydrin A3 is reacted with t-butylamine to provide hydroxyl azetidine B3. Mesylation of the hydroxyl group of azetidine B3 with methylsulfonyl chloride gives azetidine mesylate C3, which upon reacting with NaNO₂ generates mono-nitro azetidine D3. Further nitration of mono-nitro azetidine D3 with NaNO₂ in the presence of Na₂S₂O₈ and K₃Fe(CN)₆ furnishes the geminal di-nitro azetidine E3. Reaction of azetidine E3 with boron trifluoride etherate and bromoacetyl bromide provides the desired di-nitro azetidine F3. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein. Furthermore, mono-nitro compounds can be prepared by treating mono-nitro compound D3 with a Lewis Acid (e.g., boron trifluoride etherate) and acetyl bromide compound F to provide the desired mono-nitro product.

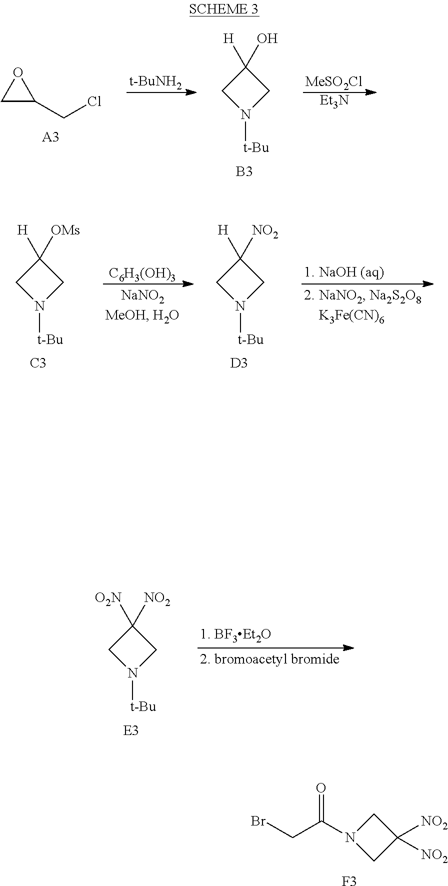

Scheme 4 illustrates an alternative exemplary procedure for preparing cyclic geminal di-nitro compounds. In the first step, heterocyclic compound A4 is reacted with an oxidant, such as pyridinium dichromate (PDC), to provide heterocyclic ketone B4. Reaction of ketone B4 with hydroxylamine gives heterocyclic oxime C₄, which upon reaction with N-bromosuccinimide (NBS) produces bromo nitro compound D4. Reaction of compound D4 with NaBH₄ furnishes mono-nitro compound E4. Reaction of mono-nitro compound E4 with NaNO₂ in the presence of Na₂S₂O₈ and K₃Fe(CN)₆ provides geminal di-nitro heterocyclic compound F4. Reaction of compound F4 with a deprotecting agent and acetyl bromide compound F provides the desired cyclic geminal di-nitro product G4. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein. Furthermore, mono-nitro compounds can be prepared by treating mono-nitro compound D4 with a deprotecting agent and acetyl bromide compound F to provide the desired mono-nitro product.

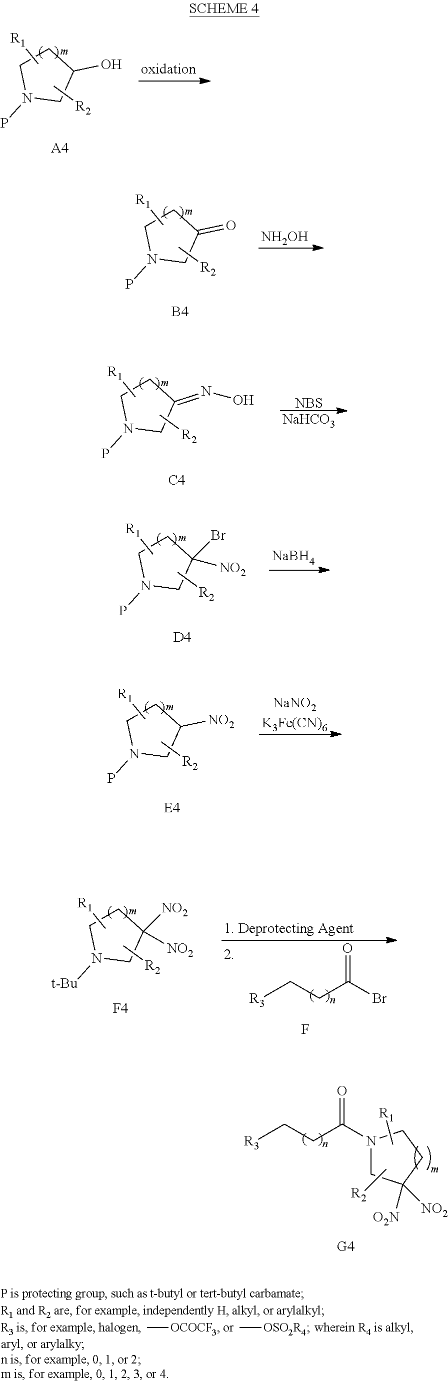

Scheme 5 illustrates yet another exemplary procedure for preparing cyclic geminal di-nitro compounds with initial steps different from those shown in Scheme 4. In the first step, heterocyclic compound A4 is reacted with methylsulfonyl chloride to provide heterocyclic mesylate B5. Reaction of mesylate B5 with NaNO₂ gives mono-nitro compound E4. Nitration of compound E4 with NaNO₂ in the presence of Na₂S₂O₈ and K₃Fe(CN)₆ provides geminal di-nitro compound F4. Reaction of compound F4 with a deprotecting agent and acetyl bromide compound F provides the desired di-nitro product G4. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein.

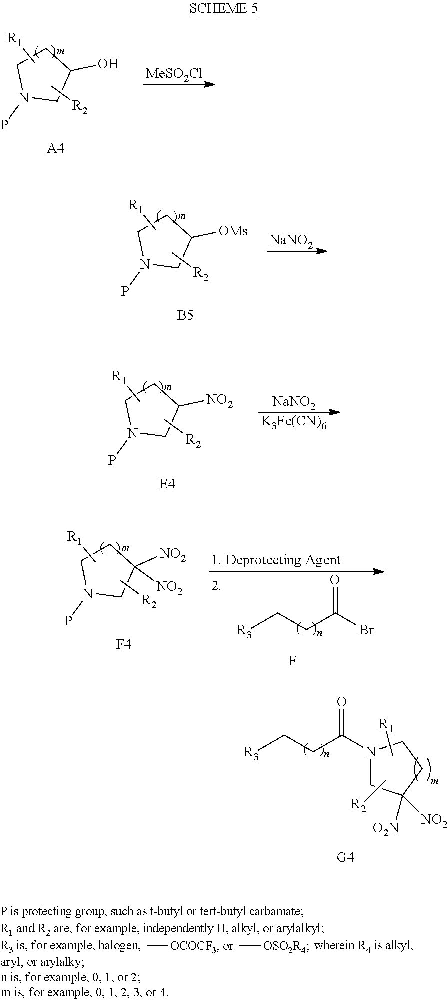

The synthetic route illustrated in Scheme 6 depicts an exemplary method for preparing cyclic vicinal di-nitro compounds. In the first step, cycloalkene A6 is reacted with N₂O₄ to provide vicinal di-nitro compound B6. Reaction of compound B6 with a deprotecting agent and acetyl bromide compound F provides the desired vicinal di-nitro product C6. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein. This synthetic procedure is contemplated to be applicable to preparing compounds having various substituents at the R₁, R₂, R₃ and R₄ positions. If a particular cycloalkene compound embraced by A6 should contain a functional group sensitive to one or more of the synthetic transformations in Scheme 6, then standard protecting group strategies are contemplated to be applied. For further description of protecting group strategies and procedures, see, for example, Greene, T. W.; Wuts, P. G. M. Protective Groups in Organic Synthesis, 2^{nd} ed.; Wiley, New York, 1991.

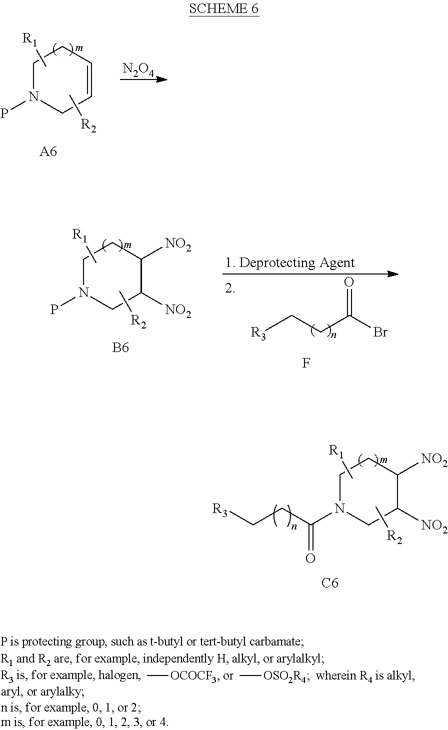

The synthetic route illustrated in Scheme 7 depicts a general method for preparing cyclic mono-nitro compounds. In the first step, chloro epoxide A7 is reacted with t-butylamine to provide hydroxy heterocyclic compound B7. Mesylation of the hydroxyl group of heterocyclic compound B7 with methylsulfonyl chloride gives mesylate C7 which upon reacting with NaNO₂ generates cyclic mono-nitro compound D7. Reaction of compound D7 with boron trifluoride etherate and acetyl bromide F provides the desired product G7. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein. This synthetic procedure illustrated in Scheme 7 is contemplated to be applicable to preparing compounds having various substituents at the R₁, R₂, R₃ and R₄ positions. If a particular epoxide compound embraced by A7 should contain a functional group sensitive to one or more of the synthetic transformations in Scheme 7, then standard protecting group strategies are contemplated to be applied. For further description of protecting group strategies and procedures, see, for example, Greene, T. W.; Wuts, P. G. M. Protective Groups in Organic Synthesis, 2^{nd} ed.; Wiley, New York, 1991.

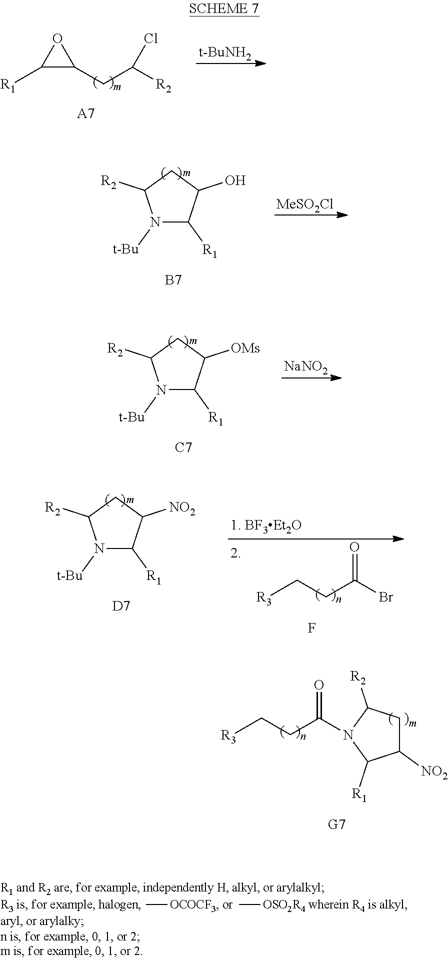

The synthetic routes described above can be modified to prepare compounds having an alkyl halide attached to the ring nitrogen atom. Exemplary synthetic procedures for preparing such compounds include reducing the amide group of compound G1-G4, G7, and C6 to an amine. Alternatively, compound F used in the procedures above could be replaced with an appropriately protected alkylhalide, such that after the alkylation reaction, the protected alkyl group attached to the ring nitrogen atom is deprotected and converted to an alkyl chloride or bromide.

Scheme 8 depicts another exemplary method for preparing cyclic mono-nitro and di-nitro compounds. Reaction of ketone B8 with hydroxylamine gives heterocyclic hydroxylamine C8, which upon reaction with N-bromosuccinimide (NBS) produces bromo nitro compound D8. Reaction of compound D8 with NaBH₄ furnishes mono-nitro compound E8. The hydroxyl protecting group (P, which may be, for example, a tert-butyldimethylsilyl group) and the 1,2-dihydroxyethane protecting group are removed using standard deprotection conditions. Exemplary deprotection conditions for removing a tert-butyldimethyl silyl group include addition of tetra-n-butylammonium fluoride. Exemplary deprotection conditions for removing a 1,2-dihydroxyethane protecting group include addition of hydrochloric acid and water. Hydroxy-ketone F8 can be converted to α-bromo ketone G8 by first reacting compound F8 with methanesulfonyl chloride to form a mesylate and then adding sodium bromide to form α-bromo ketone G8.

Di-nitro compounds can be prepared by reacting mono-nitro compound E8 with NaNO₂ in the presence of Na₂S₂O₈ and K₃Fe(CN)₆ to provide geminal di-nitro heterocyclic compound H8. The hydroxyl protecting group (P, which may be, for example, a tert-butyldiimethyl silyl group) and the 1,2-dihydroxyethane protecting group of compound H8 may be removed using standard deprotection conditions. Exemplary deprotection conditions for removing a tert-butyldiimethyl silyl group include addition of tetra-n-butylammonium fluoride. Exemplary deprotection conditions for removing a 1,2-dihydroxyethane protecting group include addition of hydrochloric acid and water. Hydroxy-ketone I8 can be converted to α-bromo ketone J8 by first reacting compound I8 with methanesulfonyl chloride to form a mesylate and then adding sodium bromide to form α-bromo ketone J8. Further description of related synthetic procedures are described in, for example, Archibald et al. in J. Org. Chem. 1990, 55, 2920-2924 and J. P. Agrawal, R. D. Hodgson, Organic Chemistry of Explosives, Wiley & Sons, England, 2007 and references cited therein.

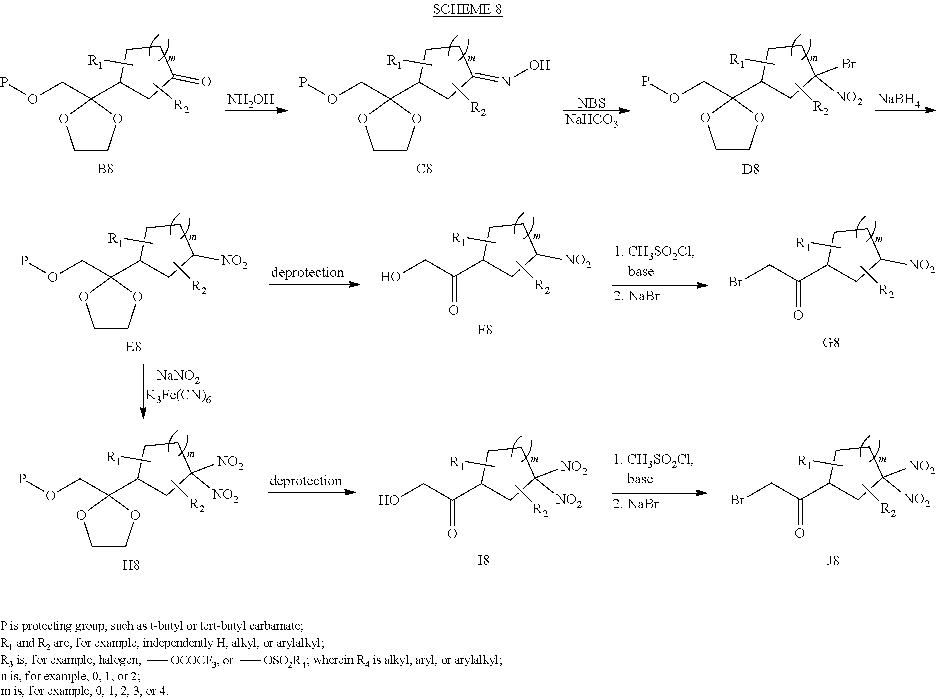

Scheme 9 illustrates an exemplary procedure for preparing acyclic geminal di-nitro compounds. In the first step, protected amino alcohol A9 is reacted with methylsulfonyl chloride to provide mesylate B9. Reaction of mesylate B9 with NaNO₂ gives mono-nitro compound E9. Nitration of compound E9 with NaNO₂ in the presence of Na₂S₂O₈ and K₃Fe(CN)₆ provides geminal di-nitro compound F9. Reaction of compound F9 with a deprotecting agent and acetyl bromide compound F provides the desired di-nitro product G9. Further description of related synthetic procedures are described in, for example, Archibald et al. in J. Org. Chem. 1990, 55, 2920-2924; U.S. Pat. No. 7,507,842; and J. P. Agrawal, R. D. Hodgson, Organic Chemistry of Explosives, Wiley & Sons, England, 2007 and references cited therein.

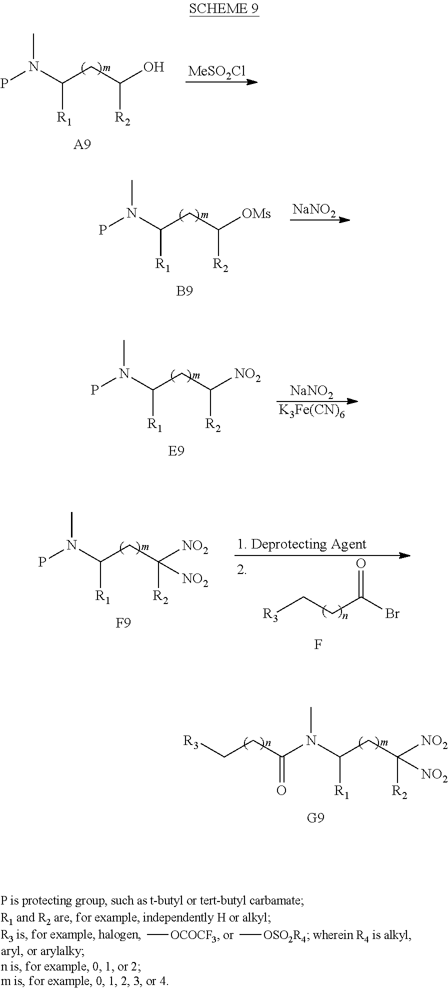

### III. Therapeutic Applications of Combination Therapy with Inorganic Nitrite Salt and Nitrite-Reductase Promoter

The invention provides methods for treating medical disorders using an inorganic nitrite salt in combination with an allosteric modulator of hemoglobin. The methods are contemplated to provide particular advantages in treating or preventing various medical disorders, such as a disorder selected from the group consisting of cancer, a cardiovascular disorder, an ischemic condition, a hemolytic condition, and a bacterial infection. Various aspects of the therapeutic methods are described in detail below.

### A. General Therapeutic Methods

The therapeutic methods described herein are particularly well-suited for treatment of diseases associated with hypoxic conditions or ischemic conditions, or otherwise may be treated or prevented using increased levels of nitric oxide. Accordingly, one aspect of the invention provides a method of treating or preventing a disorder selected from the group consisting of cancer, a cardiovascular disorder, an ischemic condition, a hemolytic condition, or a bacterial infection. The method comprises administering to a patient in need thereof a therapeutically effective amount of (i) an inorganic nitrite salt, and (ii) a nitrite reductase promoter, which preferably is an allosteric modulator of hemoglobin that promotes nitrite reductase activity.

### Exemplary Cancers

Exemplary types of cancer contemplated to be treated include brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer.

In certain embodiments, the cancer is a vascularized tumor, solid tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, bilary tract cancer, thyroid cancer, acral lentiginous melanoma, actinic keratoses, acute lymphocytic leukemia, acute myeloid leukemia, adenoid cycstic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choriod plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

The therapeutic methods may optionally comprise exposing the patient to a chemotherapeutic agent or radiation. One exemplary form of radiation is gamma rays, such as those produced from a ¹³⁷Cs source. The amount of radiation can be optimized for particular conditions. In certain embodiments, the quantity of radiation applied to the patient is at least about 2 Gy, about 5 Gy, about 10 Gy, or about 15 Gy. Exemplary chemotherapeutic agents include azacitidine, azathioprine, bleomycin, carboplatin, capecitabine, carmustine, cisplatin, chlorambucil, cyclophosphamide, cytarabine, dacarbazine, daunorubicin, docetaxel, doxifluridine, doxorubicin, epirubicin, epothilone, etoposide, fluorouracil, fulvestrant, gemcitabine, hydroxyurea, idarubicin, imatinib, lomustine, mechlorethamine, mercaptopurine, methotrexate, mitoxantrone, oxaliplatin, paclitaxel, pemetrexed, procarbazine, raloxifene, teniposide, temozolomide, thiotepa, tioguanine, tamoxifen, toremifene, valrubicin, vinblastine, vincristine, vindesine, vinorelbine, and pharmaceutically acceptable salts thereof.

### Exemplary Cardiovascular Disorders

Exemplary cardiovascular disorders include pulmonary hypertension, systemic hypertension, angina (e.g., Prinzmetal's angina), Cardiac syndrome X, myocardial infarction, peripheral artery disease, Raynaud's disease, pulmonary embolism, and intravascular thrombosis. In certain embodiments, the cardiovascular disorder is pulmonary hypertension, systemic hypertension, angina (e.g., Prinzmetal's angina), Cardiac syndrome X, myocardial infarction, peripheral artery disease, or Raynaud's disease.

### Exemplary Ischemic Conditions

Exemplary ischemic conditions include stroke, an ischemic central nervous system event, cardiac ischemia syndrome, myocardial ischemia, and tissue damage due to hypoxia.

### Exemplary Hemolytic Conditions

Exemplary hemolytic conditions include sickle cell disease (including sickle cell crisis), thalassemia, hemoglobin C disease, hemoglobin SC disease, sickle thalassemia, hereditary spherocytosis, hereditary elliptocytosis, hereditary ovalcytosis, glucose-6-phosphate deficiency and other red blood cell enzyme deficiencies, paroxysmal nocturnal hemoglobinuria (PNH), paroxysmal cold hemoglobinuria (PCH), thrombotic thrombocytopenic purpura/hemolytic uremic syndrome (TTP/HUS), idiopathic autoimmune hemolytic anemia, drug-induced immune hemolytic anemia, secondary immune hemolytic anemia, non-immune hemolytic anemia caused by chemical or physical agents, malaria, falciparum malaria, bartonellosis, babesiosis, clostridial infection, severe Haemophilus influenzae type b infection, extensive burns, transfusion reaction, rhabdomyolysis (myoglobinemia), transfusion of aged blood, cardiopulomonary bypass, and hemodialysis.

### Exemplary Bacterial Infections

The bacterial infection may be a gram-positive bacterial infection or a gram-negative bacterial infection. In certain embodiments, the bacterial infection is a gram-positive cocci bacterial infection or a gram-positive bacilli bacterial infection. In certain other embodiments, the bacterial infection is a gram-negative bacterial infection. In certain other embodiments, the bacterial infection is a gram-negative cocci bacterial infection or a gram-negative bacilli bacterial infection.

The type of bacterial infection can also be characterized according to whether the bacterial infection is caused by anaerobic or aerobic bacteria. In certain embodiments, the bacterial infection is an anaerobic bacterial infection. In certain other embodiments, the bacterial infection is an aerobic bacterial infection.

In certain embodiments, the bacterial infection is a mycobacterial infection. In more particular embodiments, the bacterial infection is an infection of bacteria selected from the group consisting of Mycobacterium tuberculosis, Staphylococcus aureus, Staphylococcus epidermidis, Enterococcus faecalis, Enterococcus faecium, Streptococcus pneumoniae, Streptococcus pyogenes, Mycobacterium smegmatis, Bacillus anthracis, Escherichia coli, Proteus mirabilis, Pseudomonas aeruginosa, Acinetobacter baumannii, Yersinia enterocolytica, Francisella tularensis, Eubacterium lentum, Bacteroides fragilis, Fusobacterium nucleatum, Porphyromonas asaccharolyticus, Clostridium perfringens, and Clostridium difficile. In still other embodiments the bacterial infection is an infection of Mycobacterium tuberculosis bacteria (abbreviated as “MTB” or “TB”).

In certain other embodiments, the bacterial infection is due to a member of the genus Peptostreptococci, a Peptostreptococci asaccharolyticus, a Peptostreptococci magnus, a Peptostreptococci micros, a Peptostreptococci prevotii, a member of the genus Porphyromonas, a Porphyromonas asaccharolytica, a Porphyromonas canoris, a Porphyromonas gingivalis, a Porphyromonas macaccae, a member of the genus Actinomyces, an Actinomyces israelii, an Actinomyces odontolyticus, a member of the genus Clostridium, a Clostridium innocuum, a Clostridium clostridioforme, a Clostridium difficile, a member of the genus Anaerobiospirillum, a member of the genus Bacteroides, a Bacteroides tectum, a Bacteroides ureolyticus, a Bacteroides gracilis (Campylobacter gracilis), a member of the genus a Prevotella, a Prevotella intermedia, a Prevotella heparinolytica, a Prevotella oris-buccae, a Prevotella bivia, a Prevotella melaninogenica, a member of the genus Fusobacterium, a Fusobacterium naviforme, a Fusobacterium necrophorum, a Fusobacterium varium, a Fusobacterium ulcerans, a Fusobacterium russii, a member of the genus Bilophila, or a Bilophila wadsworthia.

In certain other embodiments, the bacterial infection is due to an antibiotic-resistant bacteria, both aerobic and anaerobic, Gram positive and Gram negative.

### Additional Medical Conditions

Additional medical conditions contemplated for treatment or prevention using compositions described herein include nitrogen oxide related rheumatoid arthritis, diabetes (including neuropathies and vasculopathies), and systemic lupus erythematosus.

### Additional Considerations

The patient is preferably a human, such as a human suffering from a tumor. The particular combination of inorganic nitrite salt and allosteric modulator of hemoglobin may be selected according to the medical disorder suffered by the patient. For example, in certain embodiments, the inorganic nitrite salt is one of the generic or specific nitrite salts described in Section II, such as alkali metal nitrite, in particular, sodium nitrite. In certain other embodiments, the allosteric modulator of hemoglobin is one of the generic or specific allosteric modulators of hemoglobin described in Section II, such as a compound of Formula I, a compound embraced by one of the further embodiments describing definitions for certain variables of Formula I, a compound of Formula I-A, or a compound embraced by one of the further embodiments describing definitions for certain variables of Formula I-A.

Further yet, for example, with regards to Formula I, in certain embodiments, the compound corresponds to Formula I where A¹ is —C(O)—. In certain other embodiments, A¹ is —(C(R³)₂)ₓC(O)(C(R³)₂)ₓ—. In certain other embodiments, A¹ is —C(O)(C(R³)₂)ₓ—.

In certain embodiments, A² is N. In certain other embodiments, A² is —C(R⁴)—.

In certain embodiments, R¹ is halogen, —OS(O)₂R⁵, or —OC(O)CF₃. In certain other embodiments, R¹ is halogen. In certain other embodiments, R¹ is —OS(O)₂R⁵. In certain other embodiments, R¹ is —OC(O)CF₃. In certain other embodiments, R¹ is chloro, bromo, —OS(O)₂-(para-methylphenyl), —OS(O)₂CH₃, —OS(O)₂CF₃, or —OC(O)CF₃. In certain embodiments, R¹ is bromo.

In certain embodiments, m is 2. In certain other embodiments, m is 1. In certain embodiments, n is 0. In certain other embodiments, n is 1. In certain other embodiments, n is 2. In certain embodiments, p is 1. In certain other embodiments, p is 2. In certain other embodiments, p is 3.

In certain embodiments, the allosteric modulator of hemoglobin is a compound of Formula I-A:

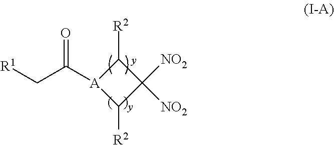

or a pharmaceutically acceptable salt or solvate thereof, wherein:
A is N or C(H);
R¹ is chloro, bromo, —OS(O)₂—(C₁-C₆alkyl), —OS(O)₂—(C₁-C₆haloalkyl), —OS(O)₂-(para-methylphenyl), or —OC(O)CF₃;
R² represents independently for each occurrence hydrogen or methyl;
y represents independently for each occurrence 1 or 2.

In certain embodiments, A is N. In certain other embodiments, A is C(H).

In certain embodiments, R¹ is chloro. In certain other embodiments, R¹ is bromo.

In certain embodiments, R¹ is —OS(O)₂—(C₁-C₆alkyl), —OS(O)₂—(C₁-C₆haloalkyl), or —OS(O)₂-(para-methylphenyl). In certain other embodiments, R¹ is —OS(O)₂CH₃, —OS(O)₂CF₃, or —OS(O)₂-(para-methylphenyl). In certain other embodiments, R¹ is —OC(O)CF₃.

In certain embodiments, R² is hydrogen or methyl. In certain embodiments, R² is hydrogen.

In certain embodiments, y is 1. In certain embodiments, one occurrence of y is 1, and the other occurrence of y is 2. In certain other embodiments, y is 2.

In certain embodiments, the allosteric modulator of hemoglobin is

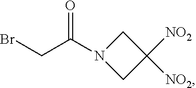 [SMILES: O=C(CBr)N1CC([N+](=O)[O-])([N+](=O)[O-])C1]

or a pharmaceutically acceptable salt or solvate thereof. In certain other embodiments, the allosteric modulator of hemoglobin is

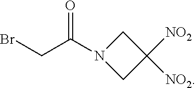 [SMILES: O=C(CBr)N1CC([N+](=O)[O-])([N+](=O)[O-])C1]

In certain other embodiments, the allosteric modulator of hemoglobin is one of the compounds listed in Tables 1 and 2 herein or a pharmaceutically acceptable salt or solvate thereof.

The description above describes multiple embodiments relating to methods of treating various disorders using an inorganic nitrite salt in combination with an allosteric modulator of hemoglobin. The patent application specifically contemplates all combinations of the embodiments. For example, the invention contemplates treating a tumor by administering a therapeutically effective amount of sodium nitrite in combination with a compound of Formula I-A wherein A is N, R¹ is chloro or bromo, and R² is hydrogen. Further, for example, the invention contemplates treating a tumor by administering a therapeutically effective amount of sodium nitrite in combination with a compound of Formula II wherein A¹ is —C(O)—, A² is N(R⁵), and R² and R³ are hydrogen.

### B. Methods of Increasing the Amount of Nitric Oxide Produced by Hemoglobin

Another aspect of the invention provides a method of increasing the amount of nitric oxide produced by hemoglobin in a patient. The method comprises administering to a patient in need thereof a therapeutically effective amount of (i) an inorganic nitrite salt, and (ii) a nitrite reductase promoter, preferably an allosteric modulator of hemoglobin that promotes nitrite reductase activity. In certain embodiments, the allosteric modulator of hemoglobin is administered at a dosage sufficient to cause a ten percent increase in the rate at which hemoglobin converts nitrite to nitric oxide in vivo. In certain other embodiments, the dose of inorganic nitrite salt and dose of allosteric modulator of hemoglobin are sufficient to cause a ten percent increase in the rate at which hemoglobin converts nitrite to nitric oxide in vivo.

### C. Methods of Preventing Sickling of a Red Blood Cell

Another aspect of the invention provides a method of preventing sickling of a red blood cell susceptible to sickling. The method comprises exposing said red blood cell to an effective amount of (i) an inorganic nitrite salt, and (ii) a nitrite reductase promoter (which preferably is an allosteric modulator of hemoglobin that promotes nitrite reductase activity) to prevent sickling of the red blood cell.

In certain embodiments, the red blood cell is a red blood cell in a patient suffering from sickle cell anemia. In certain embodiments, less than 10% of a population of said red blood cells convert to sickle form when exposed to an effective amount of (i) an inorganic nitrite salt, and (ii) an allosteric modulator of hemoglobin that promotes nitrite reductase activity, under hypoxic conditions. In certain embodiments, the hypoxic condition is characterized by a pO₂ of less than about 10 mm Hg.

### D. Dosing Amounts

Generally, the combination of pharmaceutical agents is delivered to the patient in an effective amount. Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient. The selected dosage level will depend upon a variety of factors including the activity of the particular compound of the present invention employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

In general, a suitable daily dose of a compound of the invention will be that amount of the compound which is the lowest dose effective to produce a therapeutic effect. If desired, the effective daily dose of the active compound may be administered as two, three, four, five, six or more sub-doses administered separately at appropriate intervals throughout the day, optionally, in unit dosage forms. Preferred dosing is one administration per day.

In certain embodiments, an inorganic nitrite is administered at a daily dosage of from about 0.1 μg/kg to about 10 mg/kg, about 1 μg to about 5 mg/kg, about 0.05 mg/kg to about 10 mg/kg, about 0.1 mg/kg to about 5 mg/kg, about 0.5 mg/kg to about 5 mg/kg, about 0.5 mg/kg to about 3 mg/kg, about 0.1 mg/kg to about 1.5 mg/kg, about 0.1 mg/kg to about 0.35 mg/kg, about 0.35 mg/kg to about 0.75 mg/kg, or about 0.75 mg/kg to about 1 mg/kg. In certain other embodiments, an inorganic nitrite may be administered in an amount such that the plasma concentration of nitrite ion is from about 0.05 μM to about 200 μM, about 0.1 μM to about 100 μM, about 0.5 μM to about 100 μM, about 0.1 μM to about 100 μM, or about 1 μM to about 100 μM for a desired period of time. In certain embodiments, the desired plasma concentration is maintained for a period of from about 1 hour to about 20 hours, about 1 hour to about 10 hours, or about 1 hour to about 5 hours.

### C. Combination Therapy

The therapeutic methods embrace combination therapy, which includes the administration of an inorganic nitrite salt in combination with an allosteric modulator of hemoglobin as part of a specific treatment regimen intended to provide the beneficial effect from the co-action of these therapeutic agents. Administration of these therapeutic agents in combination typically is carried out over a defined time period (e.g., hours or days depending upon the combination selected). The combination therapy may involve administration of two or more of these therapeutic agents as part of separate monotherapy regimens that result in the combinations of the present invention. Combination therapy also includes administration of these therapeutic agents in a sequential manner, that is, wherein each therapeutic agent is administered at a different time, as well as administration of these therapeutic agents, or at least two of the therapeutic agents, in a substantially simultaneous manner. Substantially simultaneous administration can be accomplished, for example, by administering to the subject a single capsule having a fixed ratio of each therapeutic agent or in multiple, single capsules for each of the therapeutic agents. Sequential or substantially simultaneous administration of each therapeutic agent can be effected by any appropriate route including, but not limited to, oral routes, intravenous routes, intramuscular routes, and direct absorption through mucous membrane tissues.

It is understood that the therapeutic agents can be administered by the same route or by different routes. For example, a first therapeutic agent of the combination selected may be administered by intravenous administration while the other therapeutic agent(s) of the combination may be administered orally. Alternatively, for example, all therapeutic agents may be administered orally or all therapeutic agents may be administered by intravenous injection.

### IV. Treating Patients with Reduced Blood Volume and/or in Need of Transfusion

One aspect of the invention provides a method of treating a patient suffering from reduced blood volume. The method comprises administering to a patient in need thereof a blood product by injection and a therapeutic agent selected from the group consisting of an organonitro compound of Formula I, organonitro compound of Formula II, hemoglobin conjugate of Formula III, hemoglobin conjugate of Formula IV, and an erythrocyte cell that has been exposed to an organonitro compound of Formula I or II; wherein Formula I is represented by:

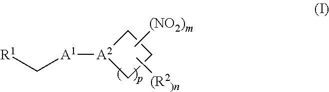

or a pharmaceutically acceptable salt or solvate thereof, wherein:
A¹ is —C(O)— or —(C(R³)₂)ₓC(O)(C(R³)₂)ₓ—;
A² is N or —C(R⁴)—;
R¹ is halogen, —OS(O)₂R⁵, or —OC(O)CF₃;
R² is C₁-C₆alkyl;
R³ and R⁴ each represent independently for each occurrence hydrogen or C₁-C₅alkyl;
R⁵ is C₁-C₆alkyl, C₁-C₆haloalkyl, aryl, or aralkyl;
m and p are independently 1, 2, or 3; and
n and x each represent independently for each occurrence 0, 1, 2, or 3;
Formula II is represented by:

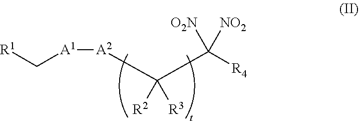

or a pharmaceutically acceptable salt or solvate thereof: wherein:
A¹ is —C(O)— or —(C(R⁵)₂)ₓC(O)(C(R⁵)₂)ₓ—;
A² is —N(R⁵)— or —C(R²)(R³)—.
R¹ is halogen, —OS(O)₂R⁶, or —OC(O)CF₃;
R² and R³ each represent independently for each occurrence hydrogen or C₁-C₆alkyl; or
R² and R³ are taken together with the carbon atom to which they are attached to form a 3-6 membered, saturated carbocyclic ring;
R⁴ is hydrogen or C₁-C₆alkyl;
R⁵ represents independently for each occurrence hydrogen or C₁-C₆alkyl;
R⁶ is C₁-C₆alkyl, C₁-C₆haloalkyl, aryl, or aralkyl;
t is an integer in the range from 1 to 12; and
x represents independently for each occurrence 0, 1, 2, or 3;
Formula III is represented by:

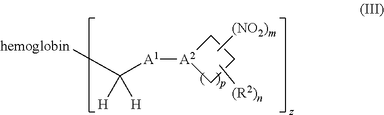

or a pharmaceutically acceptable salt or solvate thereof, wherein:
A¹ is —C(O)— or —C(O)(C(R³)₂)ₓ—;
A² is N or —C(R⁴)—;
R² is C₁-C₆alkyl;
R³ and R⁴ each represent independently for each occurrence hydrogen or C₁-C₅alkyl;
m and p are independently 1, 2, or 3;
n is 0, 1, 2, or 3;
x is 1, 2, or 3; and
z is an integer from 1 to 10; and
Formula IV is represented by:

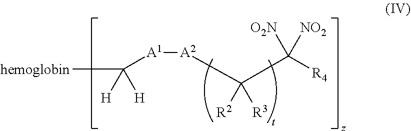

or a pharmaceutically acceptable salt or solvate thereof, wherein:
A¹ is —C(O)— or —C(O)(C(R⁵)₂)ₓ—;
A² is —N(R⁵)— or —C(R²)(R³)—.
R² and R³ each represent independently for each occurrence hydrogen or C₁-C₆alkyl; or
R² and R³ are taken together with the carbon atom to which they are attached to form a 3-6 membered, saturated carbocyclic ring;
R⁴ is hydrogen or C₁-C₆alkyl;
R⁵ represents independently for each occurrence hydrogen or C₁-C₆alkyl;
t is an integer in the range from 1 to 12;
x is 1, 2, or 3; and
z is an integer from 1 to 10.

In certain embodiments, the patient suffering from reduced blood volume is suffering from hemorrhagic shock. Hemorrhagic shock is characterized by rapid and significant loss of blood (hypovolemia), resulting in the inadequate delivery of oxygen and nutrients to meet metabolic demands. Compensatory mechanisms are often activated to preserve perfusion selectively to the brain and heart at the expense of other organ systems with progressive development of shock at the cellular and tissue level due to blood flow redistribution. The present method provides a treatment for such hemorrhagic shock.

Another aspect of the invention provides a method of performing a blood transfusion to a patient. The method comprises administering to a patient in need thereof a blood product by injection and a therapeutic agent selected from the group consisting of an organonitro compound of Formula I, organonitro compound of Formula II, hemoglobin conjugate of Formula III, hemoglobin conjugate of Formula IV, and an erythrocyte cell that has been exposed to an organonitro compound of Formula I or II, wherein Formula I is represented by:

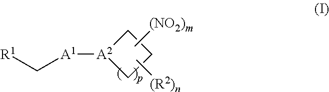

or a pharmaceutically acceptable salt or solvate thereof, wherein:
A¹ is —C(O)— or —(C(R³)₂)ₓC(O)(C(R³)₂)ₓ—;
A² is N or —C(R⁴)—;
R¹ is halogen, —OS(O)₂R⁵, or —OC(O)CF₃;
R² is C₁-C₆alkyl;
R³ and R⁴ each represent independently for each occurrence hydrogen or C₁-C₅alkyl;
R⁵ is C₁-C₆alkyl, C₁-C₆haloalkyl, aryl, or aralkyl;
m and p are independently 1, 2, or 3; and
n and x each represent independently for each occurrence 0, 1, 2, or 3;
Formula II is represented by:

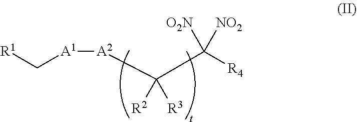

or a pharmaceutically acceptable salt or solvate thereof: wherein:
A¹ is —C(O)— or —(C(R⁵)₂)ₓC(O)(C(R⁵)₂)ₓ—;
A² is —N(R⁵)— or —C(R²)(R³)—.
R¹ is halogen, —OS(O)₂R⁶, or —OC(O)CF₃;
R² and R³ each represent independently for each occurrence hydrogen or C₁-C₆alkyl; or
R² and R³ are taken together with the carbon atom to which they are attached to form a 3-6 membered, saturated carbocyclic ring;
R⁴ is hydrogen or C₁-C₆alkyl;
R⁵ represents independently for each occurrence hydrogen or C₁-C₆alkyl;
R⁶ is C₁-C₆alkyl, C₁-C₆haloalkyl, aryl, or aralkyl;
t is an integer in the range from 1 to 12; and
x represents independently for each occurrence 0, 1, 2, or 3;
Formula III is represented by:

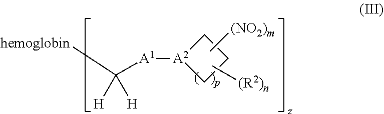

or a pharmaceutically acceptable salt or solvate thereof, wherein:
A¹ is —C(O)— or —C(O)(C(R³)₂)ₓ—;
A² is N or —C(R⁴)—;
R² is C₁-C₆alkyl;
R³ and R⁴ each represent independently for each occurrence hydrogen or C₁-C₅alkyl;
m and p are independently 1, 2, or 3;
n is 0, 1, 2, or 3;
x is 1, 2, or 3; and
z is an integer from 1 to 10; and

Formula IV is represented by:

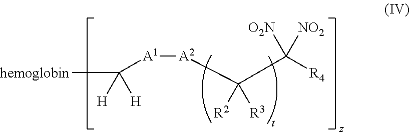

or a pharmaceutically acceptable salt or solvate thereof, wherein:
A¹ is —C(O)— or —C(O)(C(R⁵)₂)ₓ—;
A² is —N(R⁵)— or —C(R²)(R³)—.
R² and R³ each represent independently for each occurrence hydrogen or C₁-C₆alkyl; or
R² and R³ are taken together with the carbon atom to which they are attached to form a 3-6 membered, saturated carbocyclic ring;
R⁴ is hydrogen or C₁-C₆alkyl;
R⁵ represents independently for each occurrence hydrogen or C₁-C₆alkyl;
t is an integer in the range from 1 to 12;
x is 1, 2, or 3; and
z is an integer from 1 to 10.

In certain embodiments, the blood product comprises erythrocyte cells. In certain embodiments, the blood product comprises blood plasma. In certain other embodiments, the blood product comprises erythrocyte cells and blood plasma.

In certain other embodiments, the blood product and organonitro compound are administered to the patient concurrently.

In certain embodiments, the blood product is administered to the patient separately from the therapeutic agent.

In certain embodiments, the patient receives, by intravenous injection, a single composition comprising blood product and the therapeutic agent. In other certain embodiments, the patient receives, by intravenous injection, a single composition comprising a therapeutic agent, plasma, and erythrocyte cells. One exemplary composition is provided below in Table 1.

TABLE 1
Exemplary Composition for Intravenous Injection*

| Component | Amount |
|---|---|
| Erythrocyte cells (vol %) | 35-60 |
| Plasma (mL) | 17 |
| Anticoagulant | As needed (e.g., 4 mL) |
| Therapeutic Agent (e.g., | As needed, such as, an |
| ABDNAZ) | amount to treat |
| | hemorrhagic shock. |

*Amounts are based on a composition having a total volume of 282 mL.

In certain embodiments, the method further comprises administering an alkali metal nitrite to the patient. In other embodiments, the method further comprises administering sodium nitrite to the patient.

In certain embodiments, the patient receives a single composition comprising blood product, therapeutic agent, and an alkali metal nitrite.

In certain embodiments, the therapeutic agent is an organonitro compound of Formula I. In certain other embodiments, the therapeutic agent is an erythrocyte cell that has been exposed to an organonitro compound of Formula I, and said therapeutic agent is administered by injection (such as intravenous injection).

In certain embodiments, A¹ is —C(O)—, and A² is N.

In certain embodiments, R¹ is bromo.

In certain embodiments, n is 0, and m is 2.

The description above describes multiple embodiments relating to compounds of Formula I. The patent application specifically contemplates all combinations of the embodiments. For example, the invention contemplates a compound of Formula I wherein A¹ is —C(O)—, A² is N, R¹ is halogen, and n is 0.

In certain embodiments, the therapeutic agent is a compound of Formula I-A:

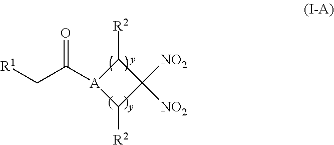

or a pharmaceutically acceptable salt or solvate thereof, wherein:
A is N or C(H);
R¹ is chloro, bromo, —OS(O)₂—(C₁-C₆alkyl), —OS(O)₂—(C₁-C₆haloalkyl), —OS(O)₂-(para-methylphenyl), or —OC(O)CF₃;
R² represents independently for each occurrence hydrogen or methyl; and
y represents independently for each occurrence 1 or 2.

In certain embodiments, the therapeutic agent is compound embraced by Formula I-A as defined by particular definitions for variables in Formula I-A, such as where A is N. In certain other embodiments, A is C(H).

In certain embodiments, R¹ is chloro or bromo. In certain embodiments, R¹ is chloro. In certain other embodiments, R¹ is bromo. In certain embodiments, R¹ is —OS(O)₂—(C₁-C₆alkyl), —OS(O)₂—(C₁-C₆haloalkyl), or —OS(O)₂-(para-methylphenyl). In certain other embodiments, R¹ is —OS(O)₂CH₃, —OS(O)₂CF₃, or —OS(O)₂-(para-methylphenyl). In certain other embodiments, R¹ is —OC(O)CF₃.

In certain embodiments, R² is hydrogen or methyl. In certain embodiments, R² is hydrogen.

In certain embodiments, y is 1. In certain embodiments, one occurrence of y is 1, and the other occurrence of y is 2. In certain other embodiments, y is 2.

The description above describes multiple embodiments relating to compounds of Formula I-A. The patent application specifically contemplates all combinations of the embodiments. For example, the invention contemplates a compound of Formula I-A wherein A is N, R¹ is chloro or bromo, and R² is hydrogen.

In certain embodiments, the therapeutic agent is

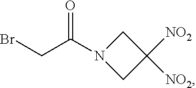 [SMILES: O=C(CBr)N1CC([N+](=O)[O-])([N+](=O)[O-])C1]

or a pharmaceutically acceptable salt thereof.

In certain embodiments, the therapeutic agent an organonitro compound embraced by Formula II as defined by particular definitions for variables in Formula II, such as where A¹ is —C(O)—. In certain other embodiments, A¹ is —(C(R⁵)₂)ₓC(O)(C(R⁵)₂)ₓ—. In certain other embodiments, A¹ is —C(O)(C(R⁵)₂)ₓ—.

In certain embodiments, A² is —N(R⁵)—. In certain other embodiments, A² is —C(R²)(R³)—.

In certain embodiments, R¹ is halogen. In certain other embodiments, R¹ is —OS(O)₂R⁶. In certain other embodiments, R¹ is —OC(O)CF₃. In certain other embodiments, R¹ is chloro, bromo, —OS(O)₂-(para-methylphenyl), —OS(O)₂CH₃, —OS(O)₂CF₃, or —OC(O)CF₃. In certain embodiments, R¹ is bromo.

In certain embodiments, R² and R³ each represent independently for each occurrence hydrogen or C₁-C₆alkyl. In certain other embodiments, R² and R³ each represent independently for each occurrence hydrogen, methyl, ethyl, or propyl. In certain other embodiments, R² and R³ each represent independently for each occurrence hydrogen or methyl. In certain embodiments, R² and R³ are hydrogen.

In certain embodiments, R⁴ is hydrogen, methyl, ethyl, propyl, butyl, or pentyl. In certain other embodiments, R⁴ is methyl, ethyl or propyl. In certain other embodiments, R⁴ is methyl.

In certain embodiments, R⁵ is hydrogen or methyl. In certain other embodiments, R⁵ is hydrogen.

In certain embodiments, R⁶ is C₁-C₆alkyl or C₁-C₆haloalkyl. In certain other embodiments, R⁶ is methyl, ethyl, or trifluoromethyl. In certain other embodiments, R⁶ is aryl, such as phenyl.

In certain embodiments, t is 1, 2, 3, 4, 5 or 6. In certain other embodiments, t is 1, 2, or 3. In certain other embodiments, t is 1. In certain embodiments, x is 1 or 2.

The description above describes multiple embodiments relating to compounds of Formula II. The patent application specifically contemplates all combinations of the embodiments. For example, the invention contemplates a compound of Formula II wherein A¹ is —C(O)—, A² is —N(R⁵)—, and R² and R³ are hydrogen.

In certain embodiments, the therapeutic agent is a compound of Formula II-A:

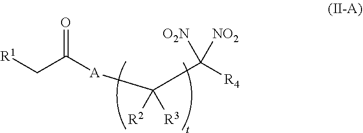

or a pharmaceutically acceptable salt or solvate thereof: wherein:
A is —N(R⁵)— or —C(R²)(R³)—.

R¹ is chloro, bromo, —OS(O)₂—(C₁-C₆alkyl), —OS(O)₂—(C₁-C₆haloalkyl), —OS(O)₂-(para-methylphenyl), or —OC(O)CF₃;
R², R³, and R⁵ each represent independently for each occurrence hydrogen or methyl;
R⁴ is hydrogen or C₁-C₆alkyl; and
t is 1, 2, or 3.

In certain embodiments, the therapeutic agent is an organonitro compound embraced by Formula II-A as defined by particular definitions for variables in Formula II-A, such as where A is —N(R⁵)—. In certain other embodiments, A is —N(CH₃)—. In certain other embodiments, A is —C(R²)(R³)—. In certain other embodiments, A is —CH₂—.

In certain embodiments, R¹ is chloro. In certain other embodiments, R¹ is bromo. In certain embodiments, R¹ is —OS(O)₂—(C₁-C₆alkyl), —OS(O)₂—(C₁-C₆haloalkyl), or —OS(O)₂-(para-methylphenyl). In certain other embodiments, R¹ is —OS(O)₂CH₃, —OS(O)₂CF₃, or —OS(O)₂-(para-methylphenyl). In certain other embodiments, R¹ is —OC(O)CF₃.

In certain embodiments, R² and R³ are hydrogen.

In certain embodiments, R⁴ is hydrogen, methyl, ethyl, propyl, butyl, or pentyl. In certain other embodiments, R⁴ is methyl, ethyl or propyl. In certain other embodiments, R⁴ is methyl.

In certain embodiments, R⁵ is hydrogen or methyl. In certain other embodiments, R⁵ is hydrogen.

The description above describes multiple embodiments relating to compounds of Formula II-A. The patent application specifically contemplates all combinations of the embodiments. For example, the invention contemplates a compound of Formula II-A wherein A is —N(R⁵)—, and R² and R³ are hydrogen.

In certain embodiments, the therapeutic agent is a hemoglobin conjugate of Formula III, and said therapeutic agent is administered by injection (such as intravenous injection).

In certain embodiments, A¹ is —C(O)— and A² is N.

In certain embodiments, n is 0, and m is 2.

In certain embodiments, the therapeutic agent is

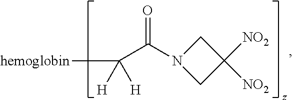

or a pharmaceutically acceptable salt thereof, where z is an integer from 1 to 10.

The therapeutic agents of Formulae I and II can be prepared based on the procedures described in Schemes 1-9 above. The hemoglobin conjugates of Formulae III and IV can be prepared by admixing hemoglobin and a therapeutic agent of Formulae I and II, respectively, to form the hemoglobin conjugate. In certain embodiments, the beta-cysteine-93 residue of hemoglobin reacts with the therapeutic agents of Formulae I and II form a thioether bond due to reaction of the thiol group of the beta-cysteine-93 residue of hemoglobin with the carbon atom bearing the R¹ group in Formulae I and II.

### V. Treating Patients with Anemia

One aspect of the invention provides a method of treating a patient suffering from anemia. The method comprises administering to a patient in need thereof a therapeutic agent selected from the group consisting of an organonitro compound of Formula I, organonitro compound of Formula II, hemoglobin conjugate of Formula III, hemoglobin conjugate of Formula IV, and an erythrocyte cell that has been exposed to an organonitro compound of Formula I or II; wherein Formula I is represented by:

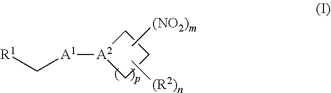

or a pharmaceutically acceptable salt or solvate thereof, wherein:
A¹ is —C(O)— or —(C(R³)₂)ₓC(O)(C(R³)₂)ₓ—;
A² is N or —C(R⁴)—;
R¹ is halogen, —OS(O)₂R⁵, or —OC(O)CF₃;
R² is C₁-C₆alkyl;
R³ and R⁴ each represent independently for each occurrence hydrogen or C₁-C₅alkyl;
R⁵ is C₁-C₆alkyl, C₁-C₆haloalkyl, aryl, or aralkyl;
m and p are independently 1, 2, or 3; and
n and x each represent independently for each occurrence 0, 1, 2, or 3;
Formula II is represented by:

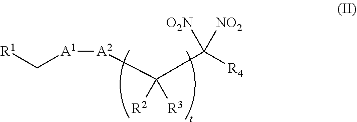

or a pharmaceutically acceptable salt or solvate thereof: wherein:
A¹ is —C(O)— or —(C(R⁵)₂)ₓC(O)(C(R⁵)₂)ₓ—;
A² is —N(R⁵)— or —C(R²)(R³)—.
R¹ is halogen, —OS(O)₂R⁶, or —OC(O)CF₃;
R² and R³ each represent independently for each occurrence hydrogen or C₁-C₆alkyl; or
R² and R³ are taken together with the carbon atom to which they are attached to form a 3-6 membered, saturated carbocyclic ring;
R⁴ is hydrogen or C₁-C₆alkyl;
R⁵ represents independently for each occurrence hydrogen or C₁-C₆alkyl;
R⁶ is C₁-C₆alkyl, C₁-C₆haloalkyl, aryl, or aralkyl;
t is an integer in the range from 1 to 12; and
x represents independently for each occurrence 0, 1, 2, or 3;
Formula III is represented by:

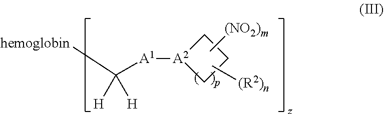

or a pharmaceutically acceptable salt or solvate thereof, wherein:
A¹ is —C(O)— or —C(O)(C(R³)₂)ₓ—;
A² is N or —C(R⁴)—;
R² is C₁-C₆alkyl;
R³ and R⁴ each represent independently for each occurrence hydrogen or C₁-C₅alkyl;
m and p are independently 1, 2, or 3;
n is 0, 1, 2, or 3;
x is 1, 2, or 3; and
z is an integer from 1 to 10; and
Formula IV is represented by:

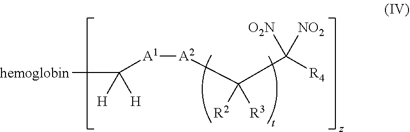

or a pharmaceutically acceptable salt or solvate thereof, wherein:
A¹ is —C(O)— or —C(O)(C(R⁵)₂)ₓ—;
A² is —N(R⁵)— or —C(R²)(R³)—.
R² and R³ each represent independently for each occurrence hydrogen or C₁-C₆alkyl; or
R² and R³ are taken together with the carbon atom to which they are attached to form a 3-6 membered, saturated carbocyclic ring;
R⁴ is hydrogen or C₁-C₆alkyl;
R⁵ represents independently for each occurrence hydrogen or C₁-C₆alkyl;
t is an integer in the range from 1 to 12;
x is 1, 2, or 3; and
z is an integer from 1 to 10.

In certain embodiments, the method further comprises administering a blood product to the patient by injection (such as intravenous injection).

In certain embodiments, the blood product comprises erythrocyte cells. In certain other embodiments, the blood product comprises blood plasma. In certain embodiments, the blood product comprises erythrocyte cells and blood plasma.

In certain embodiments, the blood product and organonitro compound are administered to the patient concurrently.

In certain other embodiments, the blood product is administered to the patient separately from the therapeutic agent.

In certain embodiments, the patient receives, by intravenous injection, a single composition comprising blood product and the therapeutic agent. In certain other embodiments, the patient receives, by intravenous injection, a single composition comprising a therapeutic agent, plasma, and erythrocyte cells. One exemplary composition is provided below in Table 2.

TABLE 2
Exemplary Composition for Intravenous Injection*

| Component | Amount |
|---|---|
| Erythrocyte cells (vol %) | 35-60 |
| Plasma (mL) | 17 |
| Anticoagulant | As needed (e.g., 4 mL) |
| Therapeutic Agent (e.g., | As needed, such as, an |
| ABDNAZ) | amount to treat |
| | hemorrhagic shock. |

*Amounts are based on a composition having a total volume of 282 mL.

In certain embodiments, the method further comprises administering an alkali metal nitrite to the patient. In other embodiments, the method further comprises administering sodium nitrite to the patient.

In certain embodiments, the therapeutic agent is an organonitro compound of Formula I. In certain other embodiments, the therapeutic agent is an erythrocyte cell that has been exposed to an organonitro compound of Formula I, and said therapeutic agent is administered by injection (such as intravenous injection).

In certain embodiments, A¹ is —C(O)—, and A² is N.

In certain embodiments, R¹ is bromo.

In certain embodiments, n is 0, and m is 2.

The description above describes multiple embodiments relating to compounds of Formula I. The patent application specifically contemplates all combinations of the embodiments. For example, the invention contemplates a compound of Formula I wherein A¹ is —C(O)—, A² is N, R¹ is halogen, and n is 0.

In certain embodiments, the therapeutic agent is a compound of Formula I-A:

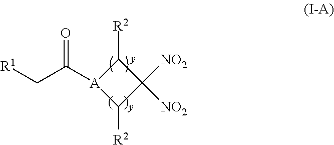

or a pharmaceutically acceptable salt or solvate thereof, wherein:
A is N or C(H);
R¹ is chloro, bromo, —OS(O)₂—(C₁-C₆alkyl), —OS(O)₂—(C₁-C₆haloalkyl), —OS(O)₂-(para-methylphenyl), or —OC(O)CF₃;
R² represents independently for each occurrence hydrogen or methyl; and
y represents independently for each occurrence 1 or 2.

In certain embodiments, the therapeutic agent is compound embraced by Formula I-A as defined by particular definitions for variables in Formula I-A, such as where A is N. In certain other embodiments, A is C(H).

In certain embodiments, R¹ is chloro or bromo. In certain embodiments, R¹ is chloro. In certain other embodiments, R¹ is bromo. In certain embodiments, R¹ is —OS(O)₂—(C₁-C₆alkyl), —OS(O)₂—(C₁-C₆haloalkyl), or —OS(O)₂-(para-methylphenyl). In certain other embodiments, R¹ is —OS(O)₂CH₃, —OS(O)₂CF₃, or —OS(O)₂-(para-methylphenyl). In certain other embodiments, R¹ is —OC(O)CF₃.

In certain embodiments, R² is hydrogen or methyl. In certain embodiments, R² is hydrogen.

In certain embodiments, y is 1. In certain embodiments, one occurrence of y is 1, and the other occurrence of y is 2. In certain other embodiments, y is 2.

The description above describes multiple embodiments relating to compounds of Formula I-A. The patent application specifically contemplates all combinations of the embodiments. For example, the invention contemplates a compound of Formula I-A wherein A is N, R¹ is chloro or bromo, and R² is hydrogen.

In certain embodiments, the therapeutic agent is

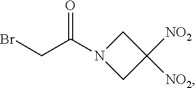 [SMILES: O=C(CBr)N1CC([N+](=O)[O-])([N+](=O)[O-])C1]

or a pharmaceutically acceptable salt thereof.

In certain embodiments, the therapeutic agent an organonitro compound embraced by Formula II as defined by particular definitions for variables in Formula II, such as where A¹ is —C(O)—. In certain other embodiments, A¹ is —(C(R⁵)₂)ₓC(O)(C(R⁵)₂)ₓ—. In certain other embodiments, A¹ is —C(O)(C(R⁵)₂)ₓ—.

In certain embodiments, A² is —N(R⁵)—. In certain other embodiments, A² is —C(R²)(R³)—.

In certain embodiments, R¹ is halogen. In certain other embodiments, R¹ is —OS(O)₂R⁶. In certain other embodiments, R¹ is —OC(O)CF₃. In certain other embodiments, R¹ is chloro, bromo, —OS(O)₂-(para-methylphenyl), —OS(O)₂CH₃, —OS(O)₂CF₃, or —OC(O)CF₃. In certain embodiments, R¹ is bromo.

In certain embodiments, R² and R³ each represent independently for each occurrence hydrogen or C₁-C₆alkyl. In certain other embodiments, R² and R³ each represent independently for each occurrence hydrogen, methyl, ethyl, or propyl. In certain other embodiments, R² and R³ each represent independently for each occurrence hydrogen or methyl. In certain embodiments, R² and R³ are hydrogen.

In certain embodiments, R⁴ is hydrogen, methyl, ethyl, propyl, butyl, or pentyl. In certain other embodiments, R⁴ is methyl, ethyl or propyl. In certain other embodiments, R⁴ is methyl.

In certain embodiments, R⁵ is hydrogen or methyl. In certain other embodiments, R⁵ is hydrogen.

In certain embodiments, R⁶ is C₁-C₆alkyl or C₁-C₆haloalkyl. In certain other embodiments, R⁶ is methyl, ethyl, or trifluoromethyl. In certain other embodiments, R⁶ is aryl, such as phenyl.

In certain embodiments, t is 1, 2, 3, 4, 5 or 6. In certain other embodiments, t is 1, 2, or 3. In certain other embodiments, t is 1. In certain embodiments, x is 1 or 2.

The description above describes multiple embodiments relating to compounds of Formula II. The patent application specifically contemplates all combinations of the embodiments. For example, the invention contemplates a compound of Formula II wherein A¹ is —C(O)—, A² is —N(R⁵)—, and R² and R³ are hydrogen.

In certain embodiments, the therapeutic agent is a compound of Formula II-A:

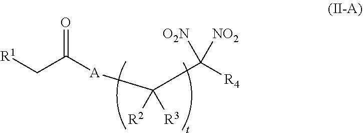

or a pharmaceutically acceptable salt or solvate thereof: wherein:
A is —N(R⁵)— or —C(R²)(R³)—.

R¹ is chloro, bromo, —OS(O)₂—(C₁-C₆alkyl), —OS(O)₂—(C₁-C₆haloalkyl), —OS(O)₂-(para-methylphenyl), or —OC(O)CF₃;
R², R³, and R⁵ each represent independently for each occurrence hydrogen or methyl;
R⁴ is hydrogen or C₁-C₆alkyl; and
t is 1, 2, or 3.

In certain embodiments, the therapeutic agent is an organonitro compound embraced by Formula II-A as defined by particular definitions for variables in Formula II-A, such as where A is —N(R⁵)—. In certain other embodiments, A is —N(CH₃)—. In certain other embodiments, A is —C(R²)(R³)—. In certain other embodiments, A is —CH₂—.

In certain embodiments, R¹ is chloro. In certain other embodiments, R¹ is bromo.

In certain embodiments, R¹ is —OS(O)₂—(C₁-C₆alkyl), —OS(O)₂—(C₁-C₆haloalkyl), or —OS(O)₂-(para-methylphenyl). In certain other embodiments, R¹ is —OS(O)₂CH₃, —OS(O)₂CF₃, or —OS(O)₂-(para-methylphenyl). In certain other embodiments, R¹ is —OC(O)CF₃.

In certain embodiments, R² and R³ are hydrogen.

In certain embodiments, R⁴ is hydrogen, methyl, ethyl, propyl, butyl, or pentyl. In certain other embodiments, R⁴ is methyl, ethyl or propyl. In certain other embodiments, R⁴ is methyl.

In certain embodiments, R⁵ is hydrogen or methyl. In certain other embodiments, R⁵ is hydrogen.

The description above describes multiple embodiments relating to compounds of Formula II-A. The patent application specifically contemplates all combinations of the embodiments. For example, the invention contemplates a compound of Formula II-A wherein A is —N(R⁵)—, and R² and R³ are hydrogen.

In certain embodiments, the therapeutic agent is a hemoglobin conjugate of Formula III, and said therapeutic agent is administered by injection (such as intravenous injection).

In certain embodiments, A¹ is —C(O)— and A² is N.

In certain embodiments, n is 0, and m is 2.

In certain embodiments, the therapeutic agent is

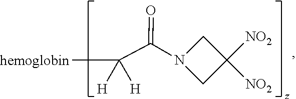

or a pharmaceutically acceptable salt thereof, where z is an integer from 1 to 10.

The therapeutic agents of Formulae I and II can be prepared based on the procedures described in Schemes 1-9 above. The hemoglobin conjugates of Formulae III and IV can be prepared by admixing hemoglobin and a therapeutic agent of Formulae I and II, respectively, to form the hemoglobin conjugate. In certain embodiments, the beta-cysteine-93 residue of hemoglobin reacts with the therapeutic agents of Formulae I and II form a thioether bond due to reaction of the thiol group of the beta-cysteine-93 residue of hemoglobin with the carbon atom bearing the R¹ group in Formulae I and II.

### VI. Preserving Blood Products

One aspect of the invention provides a method of preserving an isolated blood product. The method comprises exposing the isolated blood product to an agent selected from the group consisting of an organonitro compound of Formula I, organonitro compound of Formula II, hemoglobin conjugate of Formula III, hemoglobin conjugate of Formula IV, and an erythrocyte cell that has been exposed to an organonitro compound of Formula I or II, wherein Formula I is represented by:

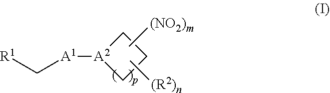

or a pharmaceutically acceptable salt or solvate thereof, wherein:
A¹ is —C(O)— or —(C(R³)₂)ₓC(O)(C(R³)₂)ₓ—;
A² is N or —C(R⁴)—;
R¹ is halogen, —OS(O)₂R⁵, or —OC(O)CF₃;
R² is C₁-C₆alkyl;
R³ and R⁴ each represent independently for each occurrence hydrogen or C₁-C₅alkyl;
R⁵ is C₁-C₆alkyl, C₁-C₆haloalkyl, aryl, or aralkyl;
m and p are independently 1, 2, or 3; and
n and x each represent independently for each occurrence 0, 1, 2, or 3;
Formula II is represented by:

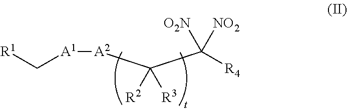

or a pharmaceutically acceptable salt or solvate thereof: wherein:
A¹ is —C(O)— or —(C(R⁵)₂)ₓC(O)(C(R⁵)₂)ₓ—;
A² is —N(R⁵)— or —C(R²)(R³)—.

R¹ is halogen, —OS(O)₂R⁶, or —OC(O)CF₃; R² and R³ each represent independently for each occurrence hydrogen or C₁-C₆alkyl; or R² and R³ are taken together with the carbon atom to which they are attached to form a 3-6 membered, saturated carbocyclic ring;
R⁴ is hydrogen or C₁-C₆alkyl;
R⁵ represents independently for each occurrence hydrogen or C₁-C₆alkyl;
R⁶ is C₁-C₆alkyl, C₁-C₆haloalkyl, aryl, or aralkyl;
t is an integer in the range from 1 to 12; and
x represents independently for each occurrence 0, 1, 2, or 3;
Formula III is represented by:

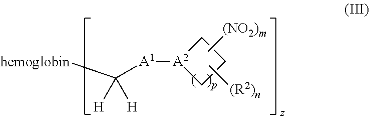

or a pharmaceutically acceptable salt or solvate thereof, wherein:
A¹ is —C(O)— or —C(O)(C(R³)₂)ₓ—;
A² is N or —C(R⁴)—;
R² is C₁-C₆alkyl;
R³ and R⁴ each represent independently for each occurrence hydrogen or C₁-C₅alkyl;
m and p are independently 1, 2, or 3;
n is 0, 1, 2, or 3;
x is 1, 2, or 3; and
z is an integer from 1 to 10; and
Formula IV is represented by:

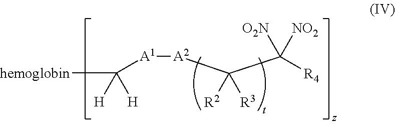

or a pharmaceutically acceptable salt or solvate thereof, wherein:
A¹ is —C(O)— or —C(O)(C(R⁵)₂)ₓ—;
A² is —N(R⁵)— or —C(R²)(R³)—.

R² and R³ each represent independently for each occurrence hydrogen or C₁-C₆alkyl; or R² and R³ are taken together with the carbon atom to which they are attached to form a 3-6 membered, saturated carbocyclic ring;
R⁴ is hydrogen or C₁-C₆alkyl;
R⁵ represents independently for each occurrence hydrogen or C₁-C₆alkyl;
t is an integer in the range from 1 to 12;
x is 1, 2, or 3; and
z is an integer from 1 to 10.

In certain embodiments, the isolated blood product is whole blood. In certain embodiments, the isolated blood product comprises erythrocyte cells. In certain other embodiments, the isolated blood product is erythrocyte cells.

In certain embodiments, the method further comprises exposing the isolated blood product to an alkali metal nitrite. In other further embodiments, the method comprises exposing the isolated blood product to sodium nitrite.

In certain embodiments, the agent is an organonitro compound of Formula I. In certain embodiments, the agent is an erythrocyte cell that has been exposed to an organonitro compound of Formula I.

In certain embodiments, A¹ is —C(O)—, and A² is N.

In certain embodiments, R¹ is bromo.

In certain embodiments, n is 0, and m is 2.

The description above describes multiple embodiments relating to compounds of Formula I. The patent application specifically contemplates all combinations of the embodiments. For example, the invention contemplates a compound of Formula I wherein A¹ is —C(O)—, A² is N, R¹ is halogen, and n is 0.

In certain embodiments, the agent is a compound of Formula I-A:

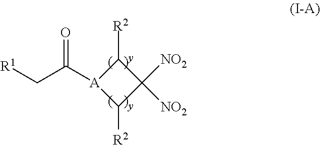

or a pharmaceutically acceptable salt or solvate thereof, wherein:
A is N or C(H);
R¹ is chloro, bromo, —OS(O)₂—(C₁-C₆alkyl), —OS(O)₂—(C₁-C₆haloalkyl), —OS(O)₂-(para-methylphenyl), or —OC(O)CF₃;
R² represents independently for each occurrence hydrogen or methyl; and
y represents independently for each occurrence 1 or 2.

In certain embodiments, the agent is compound embraced by Formula I-A as defined by particular definitions for variables in Formula I-A, such as where A is N. In certain other embodiments, A is C(H).

In certain embodiments, R¹ is chloro or bromo. In certain embodiments, R¹ is chloro. In certain other embodiments, R¹ is bromo. In certain embodiments, R¹ is —OS(O)₂—(C₁-C₆alkyl), —OS(O)₂—(C₁-C₆haloalkyl), or —OS(O)₂-(para-methylphenyl). In certain other embodiments, R¹ is —OS(O)₂CH₃, —OS(O)₂CF₃, or —OS(O)₂-(para-methylphenyl). In certain other embodiments, R¹ is —OC(O)CF₃.

In certain embodiments, R² is hydrogen or methyl. In certain embodiments, R² is hydrogen.

In certain embodiments, y is 1. In certain embodiments, one occurrence of y is 1, and the other occurrence of y is 2. In certain other embodiments, y is 2.

The description above describes multiple embodiments relating to compounds of Formula I-A. The patent application specifically contemplates all combinations of the embodiments. For example, the invention contemplates a compound of Formula I-A wherein A is N, R¹ is chloro or bromo, and R² is hydrogen.

In certain embodiments, the agent is

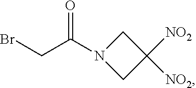 [SMILES: O=C(CBr)N1CC([N+](=O)[O-])([N+](=O)[O-])C1]

or a pharmaceutically acceptable salt thereof.

In certain embodiments, the agent an organonitro compound embraced by Formula II as defined by particular definitions for variables in Formula II, such as where A¹ is —C(O)—. In certain other embodiments, A¹ is —(C(R⁵)₂)ₓC(O)(C(R⁵)₂)ₓ—. In certain other embodiments, A¹ is —C(O)(C(R⁵)₂)ₓ—.

In certain embodiments, A² is —N(R⁵)—. In certain other embodiments, A² is —C(R²)(R³)—.

In certain embodiments, R¹ is halogen. In certain other embodiments, R¹ is —OS(O)₂R⁶. In certain other embodiments, R¹ is —OC(O)CF₃. In certain other embodiments, R¹ is chloro, bromo, —OS(O)₂-(para-methylphenyl), —OS(O)₂CH₃, —OS(O)₂CF₃, or —OC(O)CF₃. In certain embodiments, R¹ is bromo.

In certain embodiments, R² and R³ each represent independently for each occurrence hydrogen or C₁-C₆alkyl. In certain other embodiments, R² and R³ each represent independently for each occurrence hydrogen, methyl, ethyl, or propyl. In certain other embodiments, R² and R³ each represent independently for each occurrence hydrogen or methyl. In certain embodiments, R² and R³ are hydrogen.

In certain embodiments, R⁴ is hydrogen, methyl, ethyl, propyl, butyl, or pentyl. In certain other embodiments, R⁴ is methyl, ethyl or propyl. In certain other embodiments, R⁴ is methyl.

In certain embodiments, R⁵ is hydrogen or methyl. In certain other embodiments, R⁵ is hydrogen.

In certain embodiments, R⁶ is C₁-C₆alkyl or C₁-C₆haloalkyl. In certain other embodiments, R⁶ is methyl, ethyl, or trifluoromethyl. In certain other embodiments, R⁶ is aryl, such as phenyl.

In certain embodiments, t is 1, 2, 3, 4, 5 or 6. In certain other embodiments, t is 1, 2, or 3. In certain other embodiments, t is 1. In certain embodiments, x is 1 or 2.

The description above describes multiple embodiments relating to compounds of Formula II. The patent application specifically contemplates all combinations of the embodiments. For example, the invention contemplates a compound of Formula II wherein A¹ is —C(O)—, A² is —N(R⁵)—, and R² and R³ are hydrogen.

In certain embodiments, the agent is a compound of Formula II-A:

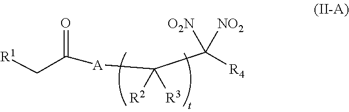

or a pharmaceutically acceptable salt or solvate thereof: wherein:
A is —N(R⁵)— or —C(R²)(R³)—.

R¹ is chloro, bromo, —OS(O)₂—(C₁-C₆alkyl), —OS(O)₂—(C₁-C₆haloalkyl), —OS(O)₂-(para-methylphenyl), or —OC(O)CF₃;
R², R³, and R⁵ each represent independently for each occurrence hydrogen or methyl;
R⁴ is hydrogen or C₁-C₆alkyl; and
t is 1, 2, or 3.

In certain embodiments, the agent is an organonitro compound embraced by Formula II-A as defined by particular definitions for variables in Formula II-A, such as where A is —N(R⁵)—. In certain other embodiments, A is —N(CH₃)—. In certain other embodiments, A is —C(R²)(R³)—. In certain other embodiments, A is —CH₂—.

In certain embodiments, R¹ is chloro. In certain other embodiments, R¹ is bromo. In certain embodiments, R¹ is —OS(O)₂—(C₁-C₆alkyl), —OS(O)₂—(C₁-C₆haloalkyl), or —OS(O)₂-(para-methylphenyl). In certain other embodiments, R¹ is —OS(O)₂CH₃, —OS(O)₂CF₃, or —OS(O)₂-(para-methylphenyl). In certain other embodiments, R¹ is —OC(O)CF₃.

In certain embodiments, R² and R³ are hydrogen.

In certain embodiments, R⁴ is hydrogen, methyl, ethyl, propyl, butyl, or pentyl. In certain other embodiments, R⁴ is methyl, ethyl or propyl. In certain other embodiments, R⁴ is methyl.

In certain embodiments, R⁵ is hydrogen or methyl. In certain other embodiments, R⁵ is hydrogen.

The description above describes multiple embodiments relating to compounds of Formula II-A. The patent application specifically contemplates all combinations of the embodiments. For example, the invention contemplates a compound of Formula II-A wherein A is —N(R⁵)—, and R² and R³ are hydrogen.

In certain embodiments, the agent is a hemoglobin conjugate of Formula III.

In certain embodiments, A¹ is —C(O)— and A² is N.

In certain embodiments, n is 0, and m is 2.

In certain embodiments, the agent is

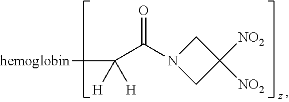

or a pharmaceutically acceptable salt thereof, where z is an integer from 1 to 10.

In certain embodiments, the agent is provided in an amount effective to extend the storage life of the blood product by at least 10%, 20% or 30% relative to the storage life of the blood product without the agent. For example, in certain embodiments, the agent is provided in an amount effective to extend the storage life of the blood product by at least 1 day, 5 days, 10 days, or 15 days.

The agents of Formulae I and II can be prepared based on the procedures described in Schemes 1-9 above. The hemoglobin conjugates of Formulae III and IV can be prepared by admixing hemoglobin and an agent of Formulae I and II, respectively, to form the hemoglobin conjugate. In certain embodiments, the beta-cysteine-93 residue of hemoglobin reacts with the agents of Formulae I and II form a thioether bond due to reaction of the thiol group of the beta-cysteine-93 residue of hemoglobin with the carbon atom bearing the R¹ group in Formulae I and II.

ABDNAZ and other compounds described herein are believed to ameliorate the well-known storage lesion that occurs with stored blood. Nitric oxide (NO) bioactivity of stored blood decreases rapidly after blood is removed from the organism, which in part limits the ability of stored blood to reverse arteriolar vasoconstriction, capillary perfusion and tissue hypoxia. These stresses consequently may affect the degree of intra and extravascular hemolysis post-transfusion. Low levels of hemoglobin (Hb) in plasma severely disrupt NO bioavailability by accelerating NO dioxygenation reactions which results in decreased NO concentration and leads to vasoconstriction. Restoration of NO bioavailability prior or concurrently with the transfusion strategy may therefore reduce the morbidity and mortality associated with blood transfusion. Furthermore, enhancing the ability of blood to generate NO by incubation with ABDNAZ or other compounds herein may decrease the number of units of blood needed for treatment and reduce healthcare costs while also extending the shelf life of packed blood.

### VII. Isolated Blood Product Compositions

Another aspect of the invention provides an isolated blood product composition. The composition comprises (i) a blood product, and (ii) an agent selected from the group consisting of an organonitro compound of Formula I, organonitro compound of Formula II, hemoglobin conjugate of Formula III, hemoglobin conjugate of Formula IV, and an erythrocyte cell that has been exposed to an organonitro compound of Formula I or II; wherein Formula I is represented by:

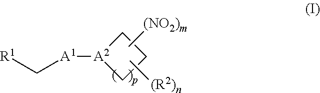

or a pharmaceutically acceptable salt or solvate thereof, wherein:
A¹ is —C(O)— or —(C(R³)₂)ₓC(O)(C(R³)₂)ₓ—;
A² is N or —C(R⁴)—;
R¹ is halogen, —OS(O)₂R⁵, or —OC(O)CF₃;
R² is C₁-C₆alkyl;
R³ and R⁴ each represent independently for each occurrence hydrogen or C₁-C₅alkyl;
R⁵ is C₁-C₆alkyl, C₁-C₆haloalkyl, aryl, or aralkyl;
m and p are independently 1, 2, or 3; and
n and x each represent independently for each occurrence 0, 1, 2, or 3;
Formula II is represented by:

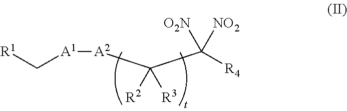

or a pharmaceutically acceptable salt or solvate thereof: wherein:
A¹ is —C(O)— or —(C(R⁵)₂)ₓC(O)(C(R⁵)₂)ₓ—;
A² is —N(R⁵)— or —C(R²)(R³)—.

R¹ is halogen, —OS(O)₂R⁶, or —OC(O)CF₃;
R² and R³ each represent independently for each occurrence hydrogen or C₁-C₆alkyl; or R² and R³ are taken together with the carbon atom to which they are attached to form a 3-6 membered, saturated carbocyclic ring;
R⁴ is hydrogen or C₁-C₆alkyl;
R⁵ represents independently for each occurrence hydrogen or C₁-C₆alkyl;
R⁶ is C₁-C₆alkyl, C₁-C₆haloalkyl, aryl, or aralkyl;
t is an integer in the range from 1 to 12; and
x represents independently for each occurrence 0, 1, 2, or 3;

Formula III is represented by:

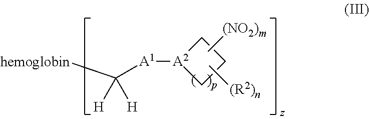

or a pharmaceutically acceptable salt or solvate thereof, wherein:
A¹ is —C(O)— or —C(O)(C(R³)₂)ₓ—;
A² is N or —C(R⁴)—;
R² is C₁-C₆alkyl;
R³ and R⁴ each represent independently for each occurrence hydrogen or C₁-C₅alkyl;
m and p are independently 1, 2, or 3;
n is 0, 1, 2, or 3;
x is 1, 2, or 3; and
z is an integer from 1 to 10; and

Formula IV is represented by:

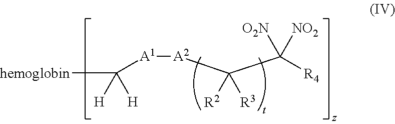

or a pharmaceutically acceptable salt or solvate thereof, wherein:
A¹ is —C(O)— or —C(O)(C(R⁵)₂)ₓ—;
A² is —N(R⁵)— or —C(R²)(R³)—.
R² and R³ each represent independently for each occurrence hydrogen or C₁-C₆alkyl; or R² and R³ are taken together with the carbon atom to which they are attached to form a 3-6 membered, saturated carbocyclic ring;
R⁴ is hydrogen or C₁-C₆alkyl;
R⁵ represents independently for each occurrence hydrogen or C₁-C₆alkyl;
t is an integer in the range from 1 to 12;
x is 1, 2, or 3; and
z is an integer from 1 to 10.

In certain embodiments, the blood product is whole blood. In certain embodiments, the blood product comprises erythrocyte cells. In certain other embodiments, the blood product comprises erythrocyte cells and blood plasma. In certain embodiments, the blood product is erythrocyte cells.

In certain embodiments, the composition further comprises an alkali metal nitrite. In certain other embodiments, the composition further comprises sodium nitrite.

The description above describes multiple embodiments relating to compounds of Formula I. The patent application specifically contemplates all combinations of the embodiments. For example, the invention contemplates a compound of Formula I wherein A¹ is —C(O)—, A² is N, R¹ is halogen, and n is 0.

In certain embodiments, the agent is a compound of Formula I-A:

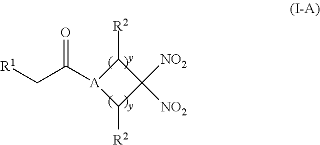

or a pharmaceutically acceptable salt or solvate thereof, wherein:
A is N or C(H);
R¹ is chloro, bromo, —OS(O)₂—(C₁-C₆alkyl), —OS(O)₂—(C₁-C₆haloalkyl), —OS(O)₂-(para-methylphenyl), or —OC(O)CF₃;
R² represents independently for each occurrence hydrogen or methyl; and
y represents independently for each occurrence 1 or 2.

In certain embodiments, the agent is compound embraced by Formula I-A as defined by particular definitions for variables in Formula I-A, such as where A is N. In certain other embodiments, A is C(H).

In certain embodiments, R¹ is chloro or bromo. In certain embodiments, R¹ is chloro. In certain other embodiments, R¹ is bromo. In certain embodiments, R¹ is —OS(O)₂—(C₁-C₆alkyl), —OS(O)₂—(C₁-C₆haloalkyl), or —OS(O)₂-(para-methylphenyl). In certain other embodiments, R¹ is —OS(O)₂CH₃, —OS(O)₂CF₃, or —OS(O)₂-(para-methylphenyl). In certain other embodiments, R¹ is —OC(O)CF₃.

In certain embodiments, R² is hydrogen or methyl. In certain embodiments, R² is hydrogen.

In certain embodiments, y is 1. In certain embodiments, one occurrence of y is 1, and the other occurrence of y is 2. In certain other embodiments, y is 2.

The description above describes multiple embodiments relating to compounds of Formula I-A. The patent application specifically contemplates all combinations of the embodiments. For example, the invention contemplates a compound of Formula I-A wherein A is N, R¹ is chloro or bromo, and R² is hydrogen.

In certain embodiments, the agent is

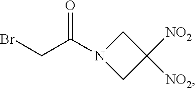 [SMILES: O=C(CBr)N1CC([N+](=O)[O-])([N+](=O)[O-])C1]

or a pharmaceutically acceptable salt thereof.

In certain embodiments, the agent an organonitro compound embraced by Formula II as defined by particular definitions for variables in Formula II, such as where A¹ is —C(O)—. In certain other embodiments, A¹ is —(C(R⁵)₂)ₓC(O)(C(R⁵)₂)ₓ—. In certain other embodiments, A¹ is —C(O)(C(R⁵)₂)ₓ—.

In certain embodiments, A² is —N(R⁵)—. In certain other embodiments, A² is —C(R²)(R³)—.

In certain embodiments, R¹ is halogen. In certain other embodiments, R¹ is —OS(O)₂R⁶. In certain other embodiments, R¹ is —OC(O)CF₃. In certain other embodiments, R¹ is chloro, bromo, —OS(O)₂-(para-methylphenyl), —OS(O)₂CH₃, —OS(O)₂CF₃, or —OC(O)CF₃. In certain embodiments, R¹ is bromo.

In certain embodiments, R² and R³ each represent independently for each occurrence hydrogen or C₁-C₆alkyl. In certain other embodiments, R² and R³ each represent independently for each occurrence hydrogen, methyl, ethyl, or propyl. In certain other embodiments, R² and R³ each represent independently for each occurrence hydrogen or methyl. In certain embodiments, R² and R³ are hydrogen.

In certain embodiments, R⁴ is hydrogen, methyl, ethyl, propyl, butyl, or pentyl. In certain other embodiments, R⁴ is methyl, ethyl or propyl. In certain other embodiments, R⁴ is methyl.

In certain embodiments, R⁵ is hydrogen or methyl. In certain other embodiments, R⁵ is hydrogen.

In certain embodiments, R⁶ is C₁-C₆alkyl or C₁-C₆haloalkyl. In certain other embodiments, R⁶ is methyl, ethyl, or trifluoromethyl. In certain other embodiments, R⁶ is aryl, such as phenyl.

In certain embodiments, t is 1, 2, 3, 4, 5 or 6. In certain other embodiments, t is 1, 2, or 3. In certain other embodiments, t is 1. In certain embodiments, x is 1 or 2.

The description above describes multiple embodiments relating to compounds of Formula II. The patent application specifically contemplates all combinations of the embodiments. For example, the invention contemplates a compound of Formula II wherein A¹ is —C(O)—, A² is —N(R⁵)—, and R² and R³ are hydrogen.

In certain embodiments, the agent is a compound of Formula II-A:

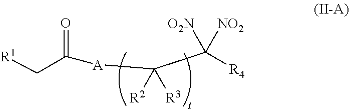

or a pharmaceutically acceptable salt or solvate thereof: wherein:
A is —N(R⁵)— or —C(R²)(R³)—.
R¹ is chloro, bromo, —OS(O)₂—(C₁-C₆alkyl), —OS(O)₂—(C₁-C₆haloalkyl), —OS(O)₂-(para-methylphenyl), or —OC(O)CF₃;
R², R³, and R⁵ each represent independently for each occurrence hydrogen or methyl;
R⁴ is hydrogen or C₁-C₆alkyl; and
t is 1, 2, or 3.

In certain embodiments, the agent is an organonitro compound embraced by Formula II-A as defined by particular definitions for variables in Formula II-A, such as where A is —N(R⁵)—. In certain other embodiments, A is —N(CH₃)—. In certain other embodiments, A is —C(R²)(R³)—. In certain other embodiments, A is —CH₂—.

In certain embodiments, R¹ is chloro. In certain other embodiments, R¹ is bromo. In certain embodiments, R¹ is —OS(O)₂—(C₁-C₆alkyl), —OS(O)₂—(C₁-C₆haloalkyl), or —OS(O)₂-(para-methylphenyl). In certain other embodiments, R¹ is —OS(O)₂CH₃, —OS(O)₂CF₃, or —OS(O)₂-(para-methylphenyl). In certain other embodiments, R¹ is —OC(O)CF₃.

In certain embodiments, R² and R³ are hydrogen.

In certain embodiments, R⁴ is hydrogen, methyl, ethyl, propyl, butyl, or pentyl. In certain other embodiments, R⁴ is methyl, ethyl or propyl. In certain other embodiments, R⁴ is methyl.

In certain embodiments, R⁵ is hydrogen or methyl. In certain other embodiments, R⁵ is hydrogen.

The description above describes multiple embodiments relating to compounds of Formula II-A. The patent application specifically contemplates all combinations of the embodiments. For example, the invention contemplates a compound of Formula II-A wherein A is —N(R⁵)—, and R² and R³ are hydrogen.

In certain embodiments, the agent is a hemoglobin conjugate of Formula III.

In certain embodiments, A¹ is —C(O)— and A² is N.

In certain embodiments, n is 0, and m is 2.

In certain embodiments, the agent is

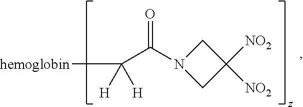

or a pharmaceutically acceptable salt thereof, where z is an integer from 1 to 10.

The agents of Formulae I and II can be prepared based on the procedures described in Schemes 1-9 above. The hemoglobin conjugates of Formulae III and IV can be prepared by admixing hemoglobin and an agent of Formulae I and II, respectively, to form the hemoglobin conjugate. In certain embodiments, the beta-cysteine-93 residue of hemoglobin reacts with the agents of Formulae I and II form a thioether bond due to reaction of the thiol group of the beta-cysteine-93 residue of hemoglobin with the carbon atom bearing the R¹ group in Formulae I and II.

In certain embodiments, the isolated blood product composition comprises a compound of Formula II, plasma, and erythrocyte cells. In certain other embodiments, the isolated blood product composition has the features provided below in Table 3.

TABLE 3
Exemplary Isolated Blood Product Composition*

| Component | Amount |
|---|---|
| Erythrocyte cells (vol %) | 35-60 |
| Plasma (mL) | 17 |
| Anticoagulant | As needed (e.g., 4 mL) |
| Therapeutic Agent (e.g., | As needed, such as, an |
| ABDNAZ) | amount to treat |
| | hemorrhagic shock. |

*Amounts are based on a composition having a total volume of 282 mL.

### VIII. Hemoglobin Conjugates

Another aspect of the invention provides an isolated hemoglobin conjugate represented by Formula III or IV:

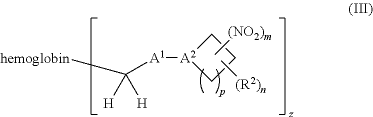

or a pharmaceutically acceptable salt or solvate thereof, wherein:
A¹ is —C(O)— or —C(O)(C(R³)₂)ₓ—;
A² is N or —C(R⁴)—;
R² is C₁-C₆alkyl;
R³ and R⁴ each represent independently for each occurrence hydrogen or C₁-C₅alkyl;
m and p are independently 1, 2, or 3;
n is 0, 1, 2, or 3;
x is 1, 2, or 3; and
z is an integer from 1 to 10; and

Formula IV is represented by:

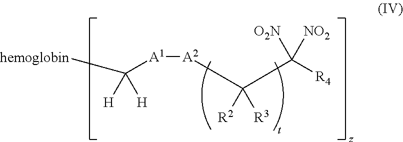

or a pharmaceutically acceptable salt or solvate thereof, wherein:
A¹ is —C(O)— or —C(O)(C(R⁵)₂)ₓ—;
A² is —N(R⁵)— or —C(R²)(R³)—.
R² and R³ each represent independently for each occurrence hydrogen or C₁-C₆alkyl; or R² and R³ are taken together with the carbon atom to which they are attached to form a 3-6 membered, saturated carbocyclic ring; R⁴ is hydrogen or C₁-C₆alkyl;
R⁵ represents independently for each occurrence hydrogen or C₁-C₆alkyl;
t is an integer in the range from 1 to 12;
x is 1, 2, or 3; and
z is an integer from 1 to 10.

In certain embodiments, the isolated hemoglobin conjugate is represented by Formula III.

In certain embodiments, A¹ is —C(O)—, and A² is N.

In certain embodiments, n is 0, and m is 2.

In certain embodiments, the isolated hemoglobin conjugate is

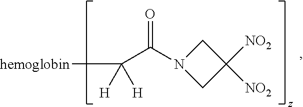

or a pharmaceutically acceptable salt thereof, wherein z is an integer from 1 to 10.

The isolated hemoglobin conjugates of Formulae III and IV can be prepared by admixing hemoglobin and an agent of Formulae I and II, respectively, to form the isolated hemoglobin conjugate. In certain embodiments, the beta-cysteine-93 residue of hemoglobin reacts with the agents of Formulae I and II form a thioether bond due to reaction of the thiol group of the beta-cysteine-93 residue of hemoglobin with the carbon atom bearing the R¹ group in Formulae I and II.

In certain embodiments, another aspect of the invention provides a pharmaceutical composition. The composition comprises a pharmaceutically acceptable carrier and an isolated hemoglobin conjugate as described herein. In certain embodiments, the pharmaceutically acceptable carrier comprises blood plasma.

### IX. Pharmaceutical Compositions

The invention provides pharmaceutical compositions. As a general matter, the pharmaceutical composition contains at least one active agent and a pharmaceutically acceptable carrier. In certain embodiments, the pharmaceutical compositions comprise an inorganic nitrite salt and/or an allosteric modulator of hemoglobin that promotes nitrite reductase activity. In certain other embodiments, the pharmaceutical compositions preferably comprise a therapeutically-effective amount of an inorganic nitrite salt and/or an allosteric modulator of hemoglobin that promotes nitrite reductase activity, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders a compound of the present invention orally bioavailable.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug administered by subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrase “parenteral administration” and “administered parenterally” as used herein means modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

### X. Kits for Use in Medical Applications

Another aspect of the invention provides a kit for treating a disorder. In certain embodiments, the kit comprises: (i) an inorganic nitrite salt, (ii) a nitrite reductase promoter (which preferably is an allosteric modulator of hemoglobin), and (iii) instructions for using the kit to treat a medical disorder.

In certain embodiments, the disorder is cancer, a cardiovascular disorder, an ischemic condition, a hemolytic condition, or a bacterial infection. In certain other embodiments, the disorder is cancer, such as a tumor. In certain other embodiments, the disorder is a cardiovascular disorder, such as pulmonary hypertension, systemic hypertension, angina, Cardiac Syndrome X, myocardial infarction, peripheral artery disease, or Raynaud's disease.

In certain embodiments, the allosteric modulator of hemoglobin is one of the generic or specific an allosteric modulators of hemoglobin described in Section II, such as a compound of Formula I, a compound embraced by one of the further embodiments describing definitions for certain variables of Formula I, a compound of Formula I-A, or a compound embraced by one of the further embodiments describing definitions for certain variables of Formula I-A. In certain embodiments, the an allosteric modulator of hemoglobin is a compound of Formula II, a compound embraced by one of the further embodiments describing definitions for certain variables of Formula II, a compound of Formula II-A, or a compound embraced by one of the further embodiments describing definitions for certain variables of Formula II-A.

The description above describes multiple aspects and embodiments of the invention, including allosteric modulators of hemoglobin, compositions comprising an allosteric modulator of hemoglobin, methods of using the allosteric modulators of hemoglobin in combination with an inorganic nitrite salt, and kits. The patent application specifically contemplates all combinations and permutations of the aspects and embodiments. For example, the invention contemplates treating tumors in a human patient by administering a therapeutically effective amount of sodium nitrite in combination with an allosteric modulator of hemoglobin of Formula I-A. Further, for example, the invention contemplates a kit for treating tumors, the kit comprising (i) an inorganic nitrite salt described herein, such as sodium nitrite, (ii) an allosteric modulator of hemoglobin, such as a compound of Formula I, and (iii) instructions for treating a tumor.

### EXAMPLES

The invention now being generally described, will be more readily understood by reference to the following examples, which are included merely for purposes of illustration of certain aspects and embodiments of the present invention, and are not intended to limit the invention.

### Example 1

The ability of ABDNAZ to increase nitric oxide production in a sample of blood under aerobic conditions and anaerobic conditions was evaluated. Experimental procedures and results are provided below.

### Experimental Procedures:

A 0.5 mL aliquot of blood was placed in a tonometer. ABDNAZ (5 μL of a 300 mM solution of ABDNAZ in dimethylsulfoxide) was optionally added to the blood sample in the Tonometer. Sodium nitrite was added to the blood sample to achieve a sodium nitrite concentration of 167 mM. Gas (either air or N₂) was passed through the Tonometer at a flow rate of 150 mL/min. Gas exiting the Tonometer was collected in three-minute intervals for a period of thirty minutes. The amount of nitric oxide in each of the collected fractions was measured by a chemiluminescent reaction with ozone in a Sievers Nitric Oxide Analyzer. The amount of nitric oxide produced from the blood sample was expressed per mole of hemoglobin present in the blood sample. All experiments were performed at 37° C.

The amount of hemoglobin (Hb) and hematocrit (Hct) in the blood sample was determined using an Advia analyzer.

### Results:

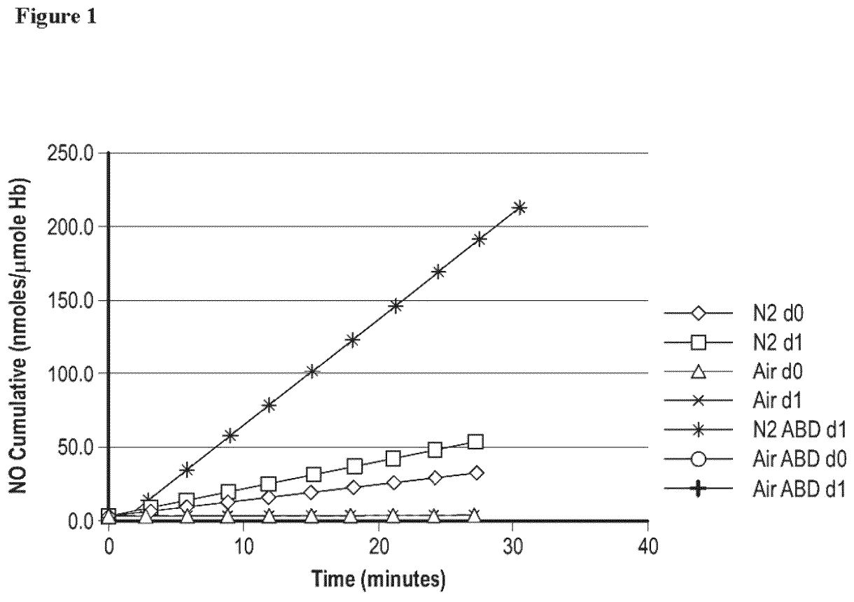

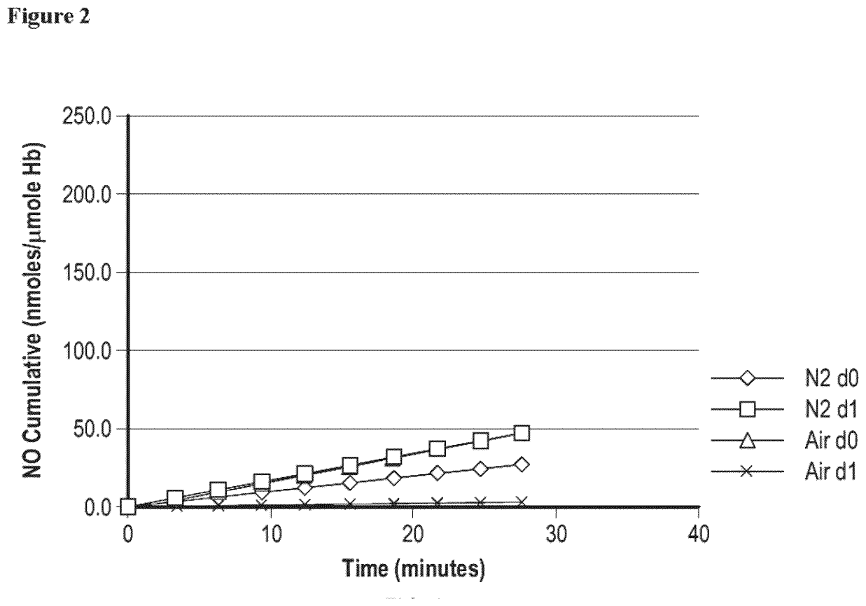

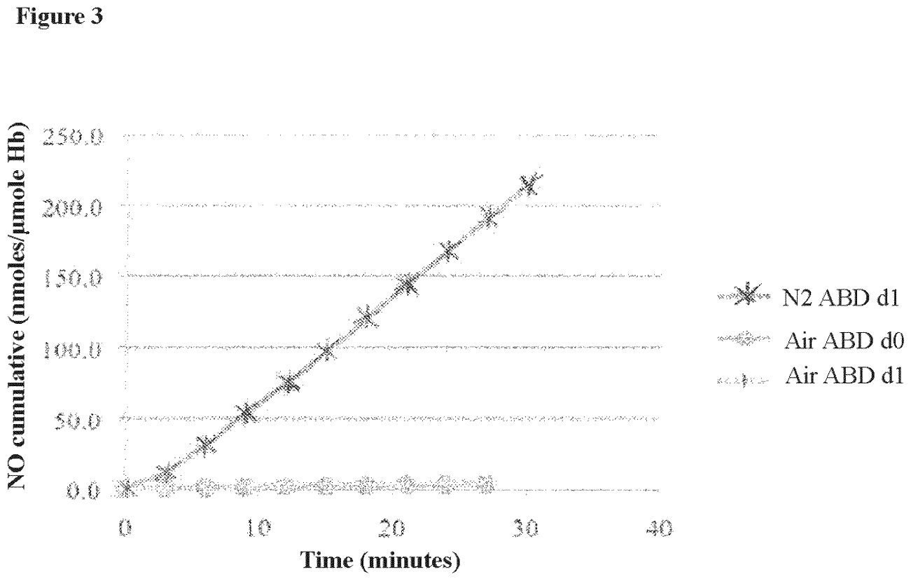

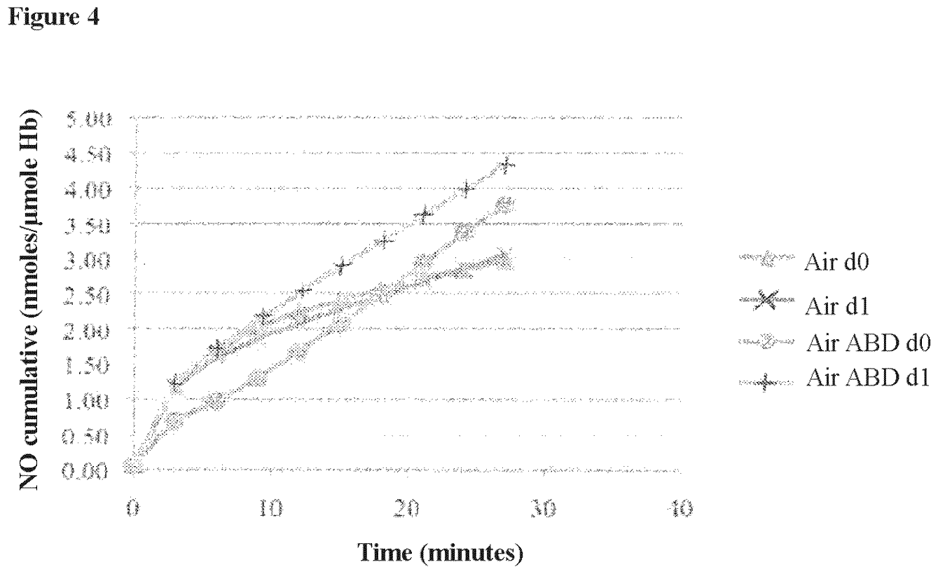

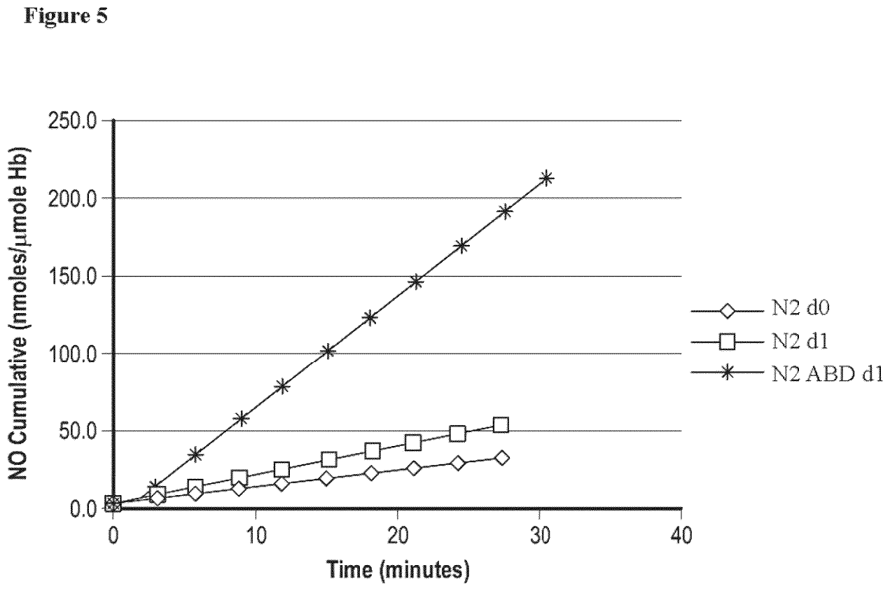

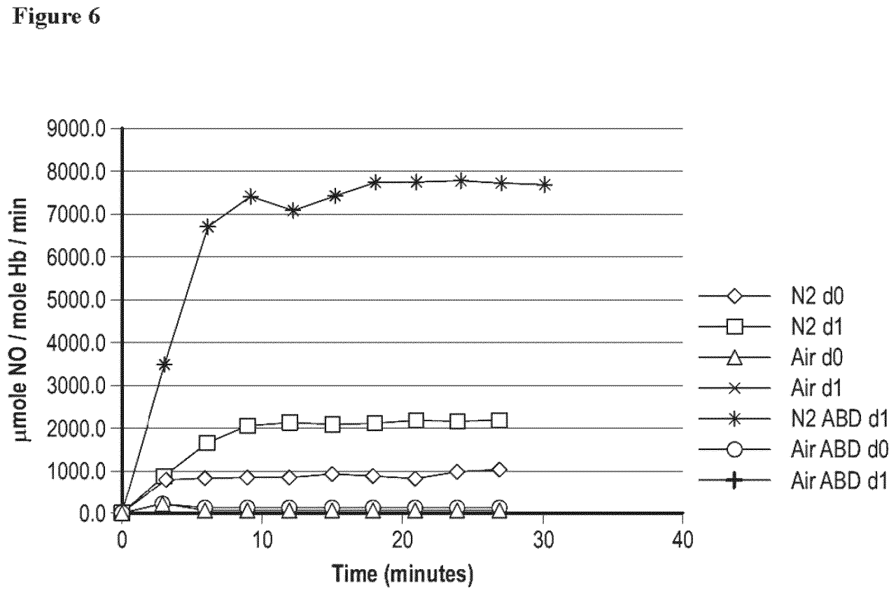

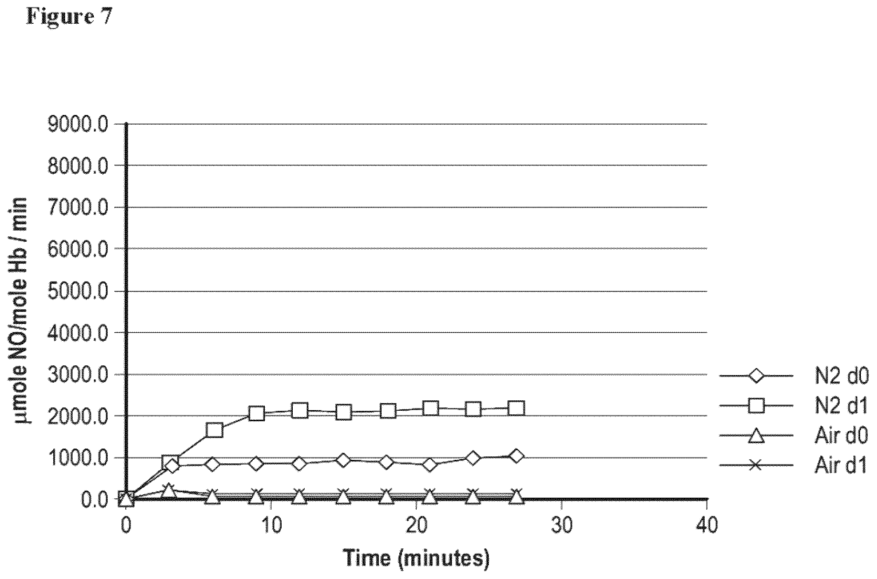

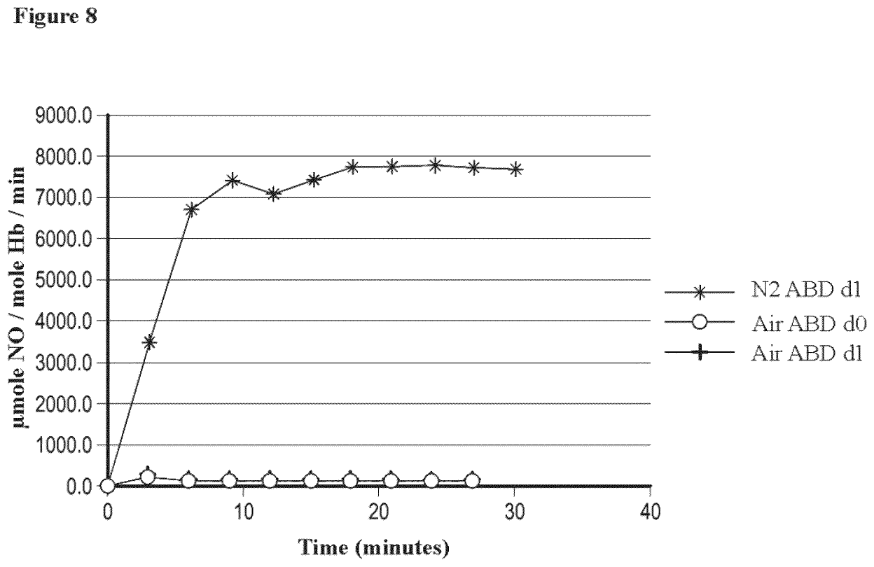

Experimental data showing the amount of nitric oxide produced by the blood samples are shown in FIGS. 1-8. In particular, FIGS. 1-5 show the cumulative amount of nitric oxide produced by the blood sample over a 30 minute time period. FIGS. 1, 3, and 5 show that exposing the blood sample to ABDNAZ under anaerobic conditions (i.e., N₂ atmosphere) resulted in a significant increase in the amount of nitric oxide produced by the blood sample, compared to the amount of nitric oxide produced (i) without ABDNAZ or (i) with ABDNAZ under aerobic conditions (i.e., air atmosphere). FIGS. 6-8 show the amount of nitric oxide formed in each three-minute period following the start of the experiment.

### Example 2

The ability of ABDNAZ to enhance blood transfusion during resuscitation from hemorrhagic shock was evaluated. Experimental procedures and results are provided below.

### Experimental Procedures:

### Animal Preparation

Experiments were performed in 55-65 g male Golden Syrian Hamsters (Charles River Laboratories, Boston, MA) fitted with a dorsal skinfold window chamber. Animal handling and care followed the NIH Guide for the Care and Use of Laboratory Animals. The hamster window chamber model is widely used for microvascular studies in the unanesthetized state. The complete surgical technique is described in detail elsewhere, such as in Colantuoni et al. in Am J Physiol 1984; 246:H508-17. Three to four days after the initial surgery, the microvasculature was examined and only animals passing an established systemic and microcirculatory inclusion criteria, as previously described (e.g., in Cabrales P. Low dose nitrite enhances perfusion after fluid resuscitation from hemorrhagic shock. Resuscitation 2009; 80:1431-6), were entered into the study.

### Acute Hemorrhage Resuscitation Protocol

Acute hemorrhage was induced by withdrawing 50% of estimated total blood volume (BV) via the carotid artery catheter within 5 min. Total BV was estimated as 7% of body weight. One hour after hemorrhage induction, animals received 25% of BV of resuscitation (200 μl/min) via the jugular vein catheter, implemented with the volume resuscitation strategy defined by the group name, according to the scheme described before.

### Experimental Groups

Animals were randomly divided into four experimental groups based on the resuscitation used, namely:
(1) Blood (group resuscitated with fresh blood only);
(2) Nitrite (group resuscitated with fresh blood followed by nitrite infusion);
(3) RRx-001 (group resuscitated with fresh blood treated with RRx-001 (i.e., ABDNAZ));
(4) RRx-001+nitrite (group resuscitated with fresh blood treated with RRx-001 (i.e., ABDNAZ) followed by nitrite infusion).

Fresh blood was collected from a donor, adult male Golden Syrian Hamsters (60-80 g). Briefly, hamster donors were anaesthetized, left carotid artery catheter was implanted, and blood was allowed to flow into heparinized tubes (sodium heparin 15 IU/mL). RBCs and plasma were separated by centrifugation (2700 rpm, 7 min). Buffy coat was discarded. RRx-001 treated cells were prepared by incubation of 1 mL of packed cells with 2 mg of RRx-001 for 30 minutes at 4° C., cells were rinsed 2× by centrifugation at 3,000 rpm with phosphate buffer saline (PBS) solution with 0.5% albumin (pre-filtered 0.22 μm, pH 7.4). After the final wash, red blood cells (RBCs) were adjusted to a 30% Hct with fresh plasma. Although not wishing to be bound by a particular theory, it is believed that RRx-001 passes through the membrane of RBCs, binds to, and modifies hemoglobin (Hb).

For the groups that received “nitrite,” 10 μM (in saline 100 μL) of sodium nitrite in saline was infused via the carotid artery catheter 10 minutes after resuscitation for the groups that received nitrite. An equal volume of saline was given to the other groups. To address effects of instrumentation and observation, an additional Sham group was included.

### Experimental Protocol

Conscious animal was placed in a restraining tube with a longitudinal slit from which the window chamber protruded, then fixed to the microscopic stage for transillumination with the intravital microscope (BX51WI, Olympus, New Hyde Park, NY). Animals were given 20 min to adjust to the tube environment before any measurements were made. The tissue image was projected onto a charge-coupled device camera (4815, COHU, San Diego, CA) connected to a videocassette recorder and viewed on a monitor. Measurements were carried out using a 40× (LUMPFL-WIR, numerical aperture 0.8, Olympus) water immersion objective. Systemic (MAP, HR, Hct, Hb, PaO₂, PaCO₂, pH, lactate, plasma nitrite, and metHb) and microvascular (arteiorlar and venular diameters, blood flow, and FCD) parameters were analyzed, as previously described (e.g., in P. Nachuraju et al. in Resuscitation 2011; 82:607-613; Cabrales P. in Resuscitation 2009; 80:1431-6; Cabrales et al. in Shock 2007; 27:380-9; and Cabrales et al. in Am J Physiol 2004; 287:H363-73) before hemorrhage (baseline), after hemorrhage (shock), and up to 90 min after volume replacement (resuscitation). Tissue viability was measured at 8 hours following hemorrhage as described previously (e.g., in Yang et al. In Invest Ophthalmol Vis Sci 2003; 44:1993-1997; and Cabrales et al. in Antioxid Redox Signal 2007; 9:375-84).

### Data Analysis

Table and figure results are presented as mean±SD. Data within each group were analyzed using analysis of variance for repeated measurements (Two-way ANOVA). When appropriate, post hoc analyses were performed with Bonferroni post-test. MetHb and Tissue viability data were analyzed using the Mann-Whitney U test. Microhemodynamic data are presented as absolute values and ratios relative to baseline values. The same vessels and capillary fields were followed so that direct comparisons to their baseline levels could be performed, allowing for more robust statistics. All statistics were calculated using GraphPad Prism 4.03 (GraphPad Software, Inc., San Diego, CA). Changes were considered statistically significant if P<0.05.

### Results:

### Systemic Response to Hemorrhage Resuscitation

Systemic hemodynamic and blood parameters are presented in Tables 1 and 2. The gold standard for treatment of hemorrhagic shock is resuscitation via blood transfusion. Thus, with the exception of blood pressure, treatment effects using RRx-001, nitrite, or both are compared to the blood only treatment group.

TABLE 1
SYSTEMIC PARAMETERS.

| | Hct | Hb | Nitrite | BP | HR |
|---|---|---|---|---|---|
| | (%) | (g/dl) | (nM) | (mmHg) | (bpm) |
| Baseline | | | | | |
| Sham | | | | 101 ± 5.6 | 406 ± 26 |
| RBCs | 50.0 ± 0.8 | 15.2 ± 0.2 | 440 ± 32 | 105.3 ± 6.4 | 416 ± 31 |
| Nitrite | 49.8 ± 0.8 | 15.2 ± 0.3 | 457 ± 23 | 104.3 ± 9.0 | 428 ± 13 |
| RRx-001 | 49.7 ± 1.0 | 15.1 ± 0.3 | 458 ± 21 | 106.3 ± 8.7 | 409 ± 21 |
| RRx-001 + | 49.1 ± 0.9 | 15.0 ± 0.3 | 439 ± 13 | 107 ± 7.4 | 429 ± 22 |
| Nitrite | | | | | |
| RRx-001 + | 28.8 ± 0.4 | 8.8 ± 0.1 | | 47.8 ± 4.4 | 453 ± 31 |
| Nitrite | | | | | |
| Resuscitation | | | | | |
| (30 min) | | | | | |
| Sham | | | | 102.5 ± 5.3 | 426 ± 42 |
| RBCs | | | | 102.4 ± 7.9 | 359 ± 36 |
| Nitrite | | | | 91.3 ± 6.9 | 438 ± 21 |
| RRx-001 | | | | 92.5 ± 6.7 | 436 ± 12 |
| RRx-001 + | | | | 88.7 ± 7.4 | 524 ± 44 |
| Nitrite | | | | | |
| Resuscitation | | | | | |
| (60 min) | | | | | |
| Sham | | | | 101.5 ± 5.2 | 425 ± 44 |
| RBCs | 31.4 ± 0.4 | 9.5 ± 0.2 | | 79.8 ± 6.3 | 373 ± 37 |
| Nitrite | 31.0 ± 0.7 | 9.5 ± 0.2 | | 96.3 ± 7.5 | 447 ± 21 |
| RRx-001 | 31.1 ± 0.6 | 9.5 ± 0.2 | | 98.5 ± 7.6 | 444 ± 10 |
| RRx-001 + | 31.1 ± 0.7 | 9.4 ± 0.2 | | 91.2 ± 9.1 | 517 ± 39 |
| Nitrite | | | | | |
| Resuscitation | | | | | |
| (90 min) | | | | | |
| Sham | | | | 101.6 ± 5.4 | 426 ± 43 |
| RBCs | 31.0 ± 0.4 | 9.5 ± 0.1 | 678 ± 39 | 106.5 ± 5.7 | 384 ± 47 |
| Nitrite | 31.5 ± 0.4 | 9.6 ± 0.1 | 791 ± 100 | 94.7 ± 6.5 | 459 ± 14 |
| RRx-001 | 31.2 ± 0.6 | 9.5 ± 0.1 | 636 ± 54 | 106.3 ± 9.0 | 453 ± 17 |
| RRx-001 + | 30.6 ± 0.4 | 9.3 ± 0.1 | 799 ± 56 | 99.1 ± 1.04 | 530 ± 43 |
| Nitrite | | | | | |

TABLE 2
ADDITIONAL SYSTEMIC PARAMETERS

| | | pO2 | pCO2 | Lactate |
|---|---|---|---|---|
| | pH | (mmHg) | (mmHg) | (mmol/L) |
| Baseline | | | | |
| Sham | | | | |
| RBCs | 7.33 ± 0.02 | 58.9 ± 1.5 | 53.3 ± 1.1 | 1.42 ± 0.19 |
| Nitrite | 7.34 ± 0.02 | 60.5 ± 1.0 | 52.9 ± 1.2 | 1.30 ± 0.13 |
| RRx-001 | 7.32 ± 0.01 | 59.9 ± 2.5 | 54.2 ± 1.8 | 1.39 ± 0.20 |
| RRx-001 + Nitrite | 7.33 ± 0.02 | 61.1 ± 1.6 | 53.7 ± 1.6 | 1.37 ± 0.19 |
| RRx-001 + Nitrite | 7.28 ± 0.02 | 98.0 ± 3.9 | 38.4 ± 1.2 | 4.19 ± 0.57 |
| Resuscitation | | | | |
| (30 min) | | | | |
| Sham | | | | |
| RBCs | | | | |
| Nitrite | | | | |
| RRx-001 | | | | |
| RRx-001 + Nitrite | | | | |
| Resuscitation | | | | |
| (60 min) | | | | |
| Sham | | | | |
| RBCs | 7.34 ± 0.01 | 69.8 ± 1.7 | 50.6 ± 1.5 | 2.08 ± 0.22 |
| Nitrite | 7.38 ± 0.02 | 76.0 ± 2.9 | 48.3 ± 2.2 | 1.95 ± 0.11 |
| RRx-001 | 7.39 ± 0.01 | 70.9 ± 3.4 | 48.0 ± 3.1 | 1.93 ± 0.23 |
| RRx-001 + Nitrite | 7.39 ± 0.02 | 79.5 ± 4.3 | 44.7 ± 2.1 | 1.41 ± 0.12 |
| Resuscitation | | | | |
| (90 min) | | | | |
| Sham | | | | |
| RBCs | 7.35 ± 0.02 | 63.1 ± 1.6 | 51.0 ± 1.8 | 1.86 ± 0.18 |
| Nitrite | 7.36 ± 0.02 | 65.2 ± 3.0 | 45.0 ± 2.4 | 1.71 ± 0.20 |
| RRx-001 | 7.36 ± 0.01 | 68.7 ± 2.9 | 49.0 ± 2.2 | 1.63 ± 0.23 |
| RRx-001 + Nitrite | 7.38 ± 0.02 | 70.4 ± 3.9 | 50.7 ± 2.7 | 1.41 ± 0.14 |

During hemorrhagic shock, Hct and Hb dropped about 50% for all treatment groups and no significant differences were seen between groups at any time during the study. Although resuscitation partially restored blood pressure (MAP) from shock in all treatment groups, MAP was significantly decreased in the RRx-001 group at 60 minutes. Compared to the blood group, significant differences in MAP and heart rate were seen in the blood+nitrite, RRx-001, and RRx-001+nitrite groups following resuscitation. For the nitrite group, MAP was significantly decreased at 30 and 90 minutes. MAP in the RRx-001, was only decreased at 30 minutes and in the RRx-001+nitrite group it was significantly decreased at 30 and 60 minutes. HR for the nitrite, RRx-001, and RRx-001+nitrite treatment groups compared to the blood group was significantly increased.

Hemorrhagic shock decreased arterial pH and pCO2, significantly compromising acid based balance relative to baseline. Resuscitation partially recovered all blood gas parameters. In the nitrite, RRx-001, and RRx-001+nitrite groups, pH was significantly increased at 60 minutes post resuscitation compared to the blood group. For the RRx-001+nitrite group, pH was also significantly increased at 90 minutes post resuscitation. pO2 levels were significantly increased: in the in the nitrite group at 60 minutes post resuscitation; in the RRx-001 group at 90 minutes post resuscitation; in the RRx-001+nitrite group, during 60 minutes of shock and at 60 and 90 minutes post resuscitation. Compared to the blood group, pCO2 levels in the RRx-001 group were not significantly different. In the RRx-001+nitrite group, lactate (lact) levels were significantly decreased at 60 and 90 minutes post-resuscitation. Decreases in lactate levels in the nitrite and RRx-001 groups did not reach significance.

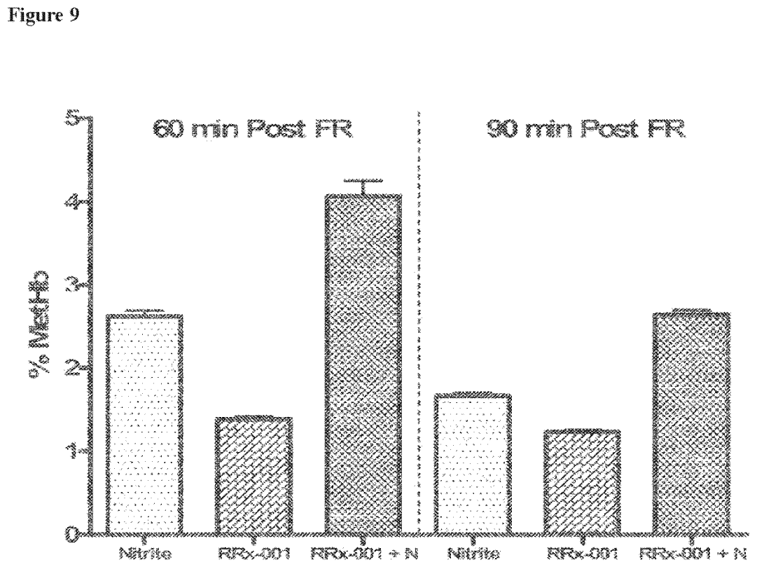

The percent MetHb levels for the nitrite, RRx-001, and RRx-001+nitrite groups are presented in FIG. 9. In normal, healthy individuals metHb levels are about 1% of the total Hb and methemoglobinemia occurs when the concentration of metHb in the blood exceeds 1.5 g/dL (8%-12% of the normal Hb level). See, for example, Hamirani et al. in Tex Heart Inst J 2008; 35:76-77. Of the three treatment groups, only RRx-001 had normal metHb levels.

### Microvascular Measurements

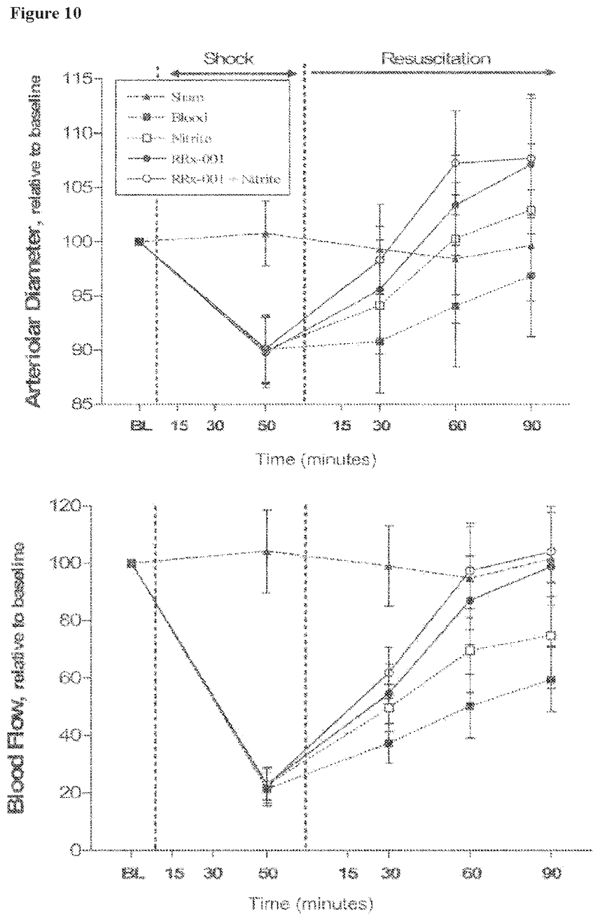

Changes in arteriolar diameter and blood flow during the hemorrhagic shock resuscitation protocol for all experimental groups are presented in FIG. 10. Compared to the blood group, arterial diameter and blood flow were increased in all treatment groups. However, these increases only reached significance in the RRx-001 and RRx-001+nitrite groups. Arteriolar diameter was significantly increased at 60 and 90 minutes (P<0.01 and P<0.05, respectively) only in RRx-001+nitrite following resuscitation and blood flow was significantly (P<0.001) increased in the both the RRx-001 and RRx-001+nitrite groups at 60 and 90 minutes. At 30 minutes post resuscitation, the difference in blood flow between the blood only and RRx-001+nitrite groups was also significant (P<0.05).

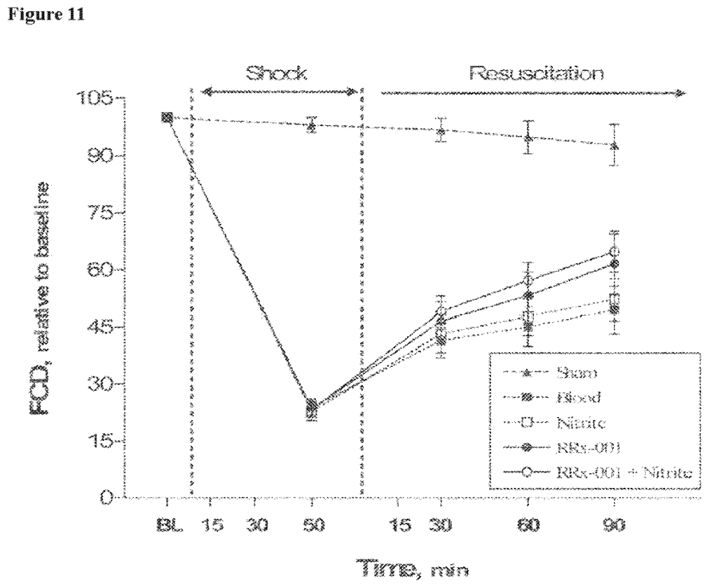

Changes in the number of capillaries perfused with RBCs during the protocol are presented in FIG. 11. Resuscitation partially restored hemorrhage induced reductions in functional capillary density (FCD) in all groups. Again, compared to the blood group, FCD was not significantly different in the nitrite group following resuscitation. However, both RRx-001 and RRx-001+nitrite treatment resulted in significant (P<0.05 and P<0.01, respectively) increases in FCD at 90 minutes post resuscitation. Statistical significance (P<0.05) was also observed at 60 minutes in the RRx-001+nitrite group.

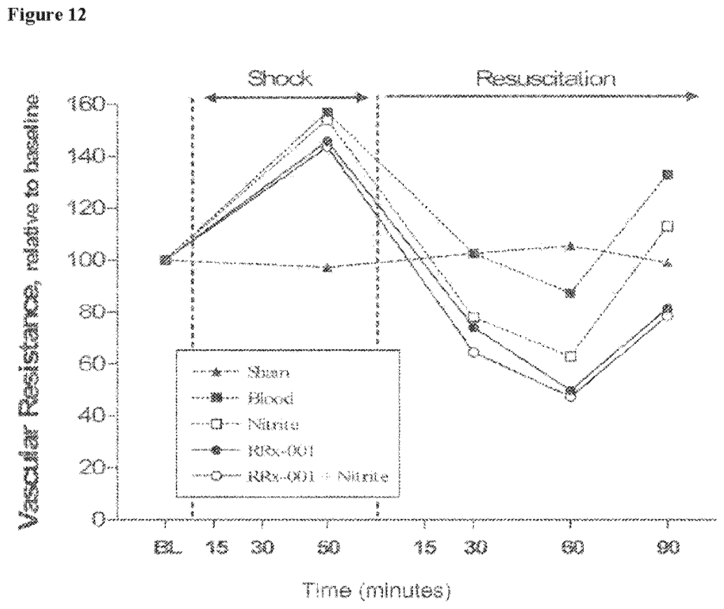

Calculated vascular resistance (average MAP/average blood flow) relative to baseline is presented in FIG. 12. Peripheral vascular resistance for all groups increased after hemorrhagic shock (about 1.5 times the resistance at baseline) and decreased after resuscitation. The RRx-001 and RRx-001+nitrite groups had a similar impact on vascular resistance.

### Tissue Viability

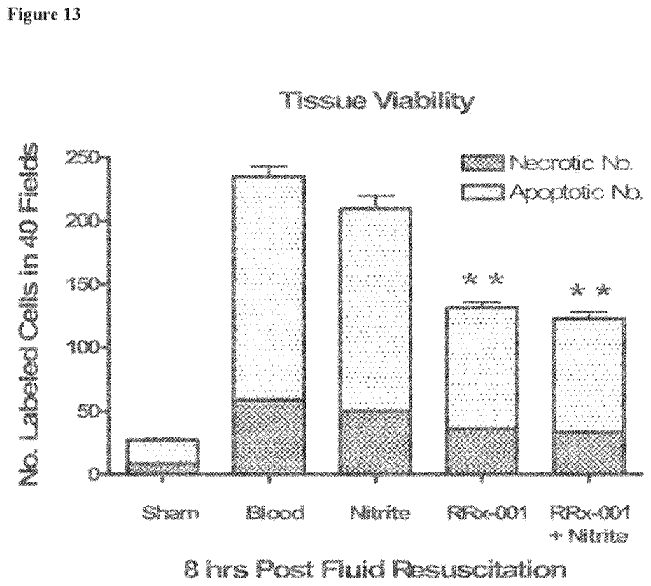

Tissue viability (the number of apoptotic and necrotic cells in 40 fields) for all treatment groups at 8 hours following resuscitation is presented in FIG. 13. The number of apoptotic cells in the RRx-001 and RRx-001+nitrite groups was significantly (P<0.01) less than the blood group. The number of necrotic cells was also significantly (P<0.01) less in the RRx-001 and RRx-001+nitrite groups compared to the blood group. Supplementation with nitrite also significantly reduced the number of necrotic cells.

### Discussion

Experimental results show that RRx-001 treated blood with or without nitrite supplementation provides superior systemic and microvascular hemodynamic responses compared to blood transfusion with or without nitrite. Incorporating RRx-001 into transfusion-based resuscitation affords the added benefit of selectively increasing NO generation under hypoxic conditions. Without being bound by a particular theory, it is believed that RRx-001 generates NO in two ways: i) as an NO donor: through metabolism of the dinitro groups released from the compound, and ii) as an NO promoter: beta-Cys-93 modification by RRx-001 enhances hypoxia-mediated nitrite reduction to NO by deoxyhemoglobin. Our results demonstrate that these RRx-001 mediated benefits improved systemic and microvascular parameters, which appears to correlate with tissue viability. Thus, RRx-001 treated blood should minimize short and long term organ damage after hemorrhagic shock.

Hemorrhagic hypotension leads to a well-characterized sequence of events, and ultimately to vascular decompensation, due to a continuous increase in peripheral vascular resistance. The outcome of hemorrhagic shock is related to the degree of hypovolemia, the magnitude of acquired oxygen debt, and the delay in treatment. Monitoring the microcirculation is crucial in determining the effect of changes in intravascular volume in tissue hypo-perfusion. Application of various techniques, including intravital microscopy, has shown the presence of major microcirculatory alterations during hemorrhage, and the persistence of these microcirculatory alterations have been associated with multiorgan failure and death. See, for example, Sinaasappel et al. in J Physiol 1999; 514(Pt 1):245-253; and Ellis et al. in Crit Care 2005; 9(Suppl 4):S3-8.

Blood transfusion is currently the gold standard for treatment of severe hemorrhagic shock. When blood is used during resuscitation, intravascular blood volume and oxygen carrying capacity are restored, cardiovascular function improves, energy requirements are met, and survival more likely. Practically however, transfusion post hemorrhage recovers the microcirculation, but not necessarily to normal levels. The injury resulting from the shock phase prior to resuscitation limits perfusion during the resuscitation and thus prevents full recovery of the microcirculation immediately post resuscitation. Moreover, when blood is used during resuscitation, “normal” MAP is restored, however restoring MAP is not necessarily accompanied by the restoration of organ perfusion and oxygenation, due to microvascular flow dysfunctions (the so-called “no reflow” phenomenon). See, for example, Zakaria et al. in J Trauma 2005; 58:499-508; and Rezkalla et al. in Circulation 2002; 105:656-62. During the shock phase and immediately post resuscitation, vascular endothelial shear stress and endothelium NO synthase (eNOS) activity is also impaired and results in delayed dilation of the endothelium. Over time, eNOS activity and microvascular flow dysfunction recover. However, if the resuscitation is inadequate during this critical period multi-organ injury can ensue. The results of our study suggest that during the time when NO synthase is still malfunctioning, incorporation of RRx-001 with blood resuscitation, via restoration of intravascular NO concentration, would increase perfusion by relaxing arterioles and lowering vascular resistance leading to improved microvascular function, reduced cell death, and preserved tissue viability, ensuring a better overall outcome compared to blood transfusion alone.

The use of NO donors under conditions of hemorrhagic shock have been shown to result in enhanced myocardial contractile activity that leads to a situation where mean arterial pressure does not decrease further despite significant decrease of total peripheral resistance. Remizova and colleagues studied the effects of an NO donor, DNIC-GS (dinitrosyl iron complexes with glutathione) in a hemorrhagic shock model. See, for example, Remizova et al. in Eur J Pharmacol 2011; 662:40-46. They found that injection of DNIC-GS into the blood flow of rats prior to hemorrhage by increased stroke volume, left ventricular work, and cardiac output. The results of our study indicate that RRx-001 should improve these indices of cardiac function in the face of decreased vascular resistance.

Nitrite, a biologic metabolite of NO, present in a variety of foods. Nitrite has been appreciated as an inflammatory mediator of nitration reactions and a precursor for NO under acidic or ischemic conditions and plasma nitrite levels correlate with eNOS activity and are tightly controlled. We have previously studied the effects of nitrite supplementation (10 μM and 50 μM nitrite) on systemic (BP, HR, pH, pO2, pCO2) and microvascular parameters (arteriolar diameter, blood flow, FCD) after resuscitation from hemorrhagic shock. Similar effects on systemic and microvascular parameters were observed with the administration of 10 μM nitrate compared to the nitrite group in the present study. By comparison, administration of 50 μM nitrite had a more profound effect on arteriolar diameter and blood flow but negatively affected blood pressure and metHb levels. Blood pressure in the 50 μM nitrite group was significantly decreased compared to 0 μM group (control group) at 60 minutes following resuscitation and at 60 and 90 minutes; % metHb was 5.8±1.8 and 3.1±1.3, respectively. In the current study, RRx-001 treatment maintained blood pressure following resuscitation and resulted in metHb levels of only 1.4±0.1 at 60 minutes and 1.2±0.1 at 90 minutes which corresponds to metHb levels in healthy individuals of about 1% of the total Hb: Methemoglobinemia occurs when the concentration of metHb in the blood exceeds 1.5 g/dL (8%-12% of the normal Hb level), where tissue oxygenation is compromised.

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating or preventing a disorder selected from the group consisting of; cancer, a cardiovascular disorder, an ischemic condition, a hemolytic condition, and a bacterial infection, the method comprising:
administering to a patient in need thereof a therapeutically effective amount of (i) an inorganic nitrite salt and (ii) an allosteric modulator of hemoglobin that promotes nitrite reductase activity.

2. The method of claim 1, wherein the disorder is cancer, wherein the cancer is a tumor, and further comprising exposing the patient to a chemotherapeutic agent or radiation.

3. (canceled)

4. (canceled)

5. The method of claim 1, wherein:
the disorder is a cardiovascular disorder or a hemolytic condition,
the cardiovascular disorder is selected from the group consisting of: pulmonary hypertension, systemic hypertension, angina, Cardiac Syndrome X, myocardial infarction, peripheral artery disease, and Raynaud's disease, and
the hemolytic condition is sickle cell disease.

6. (canceled)

7. (canceled)

8. (canceled)

9. (canceled)

10. The method of claim 1, wherein the inorganic nitrite salt is an alkali metal nitrite or is represented by NO₂—N(R′)₄, and wherein R′ represents independently for each occurrence hydrogen, optionally substituted alkyl, optionally substituted heteroalkyl, optionally substituted cycloalkyl, optionally substituted heterocycloalkyl, optionally substituted aryl, optionally substituted aralkyl, optionally substituted heteroaryl, or optionally substituted heteroaralkyl.

11. (canceled)

12. (canceled)

13. The method of claim 1, wherein the allosteric modulator of hemoglobin binds to the beta-cysteine-93 residue of hemoglobin.

14. The method of any one of claim 1, wherein the allosteric modulator of hemoglobin is a compound of Formula I or II, wherein Formula I is represented by: 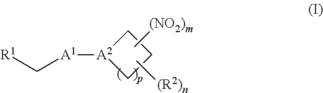
or a pharmaceutically acceptable salt or solvate thereof, wherein:
A¹ is —C(O)— or —(C(R³)₂)ₓC(O)(C(R³)₂)ₓ;
A² is N or —C(R⁴)—;
R¹ is halogen, —OS(O)₂R⁵, or —OC(O)CF₃;
R² is C₁-C₆ alkyl;
R³ and R⁴ each represent independently for each occurrence hydrogen or C₁-C₅ alkyl;
R⁵ is C₁-C₆ alkyl, C₁-C₆ haloalkyl, aryl, or aralkyl;
m and p are independently 1, 2, or 3; and
n and x each represent independently for each occurrence 0, 1, 2, or 3; and
Formula II is represented by: 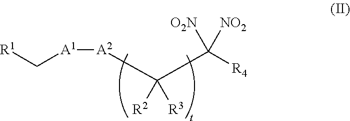
or a pharmaceutically acceptable salt or solvate thereof: wherein:
A¹ is —C(O)— or —(C(R⁵)₂)ₓC(O)(C(R⁵)₂)ₓ—;
A² is —N(R⁵)— or —C(R²)(R³)—;
R¹ is halogen, —OS(O)₂R⁶, or —OC(O)CF₃;
R² and R³ each represent independently for each occurrence hydrogen or C₁-C₆ alkyl; or
R² and R³ are taken together with the carbon atom to which they are attached to form a 3-6 membered, saturated carbocyclic ring;
R⁴ is hydrogen or C₁-C₆ alkyl;
R⁵ represents independently for each occurrence hydrogen or C₁-C₆ alkyl;
R⁶ is C₁-C₆ alkyl, C₁-C₆ haloalkyl, aryl, or aralkyl;
t is an integer in the range from 1 to 12; and
x represents independently for each occurrence 0, 1, 2, or 3.

15. The method of claim 1, wherein the allosteric modulator of hemoglobin is a compound of Formula I-A: 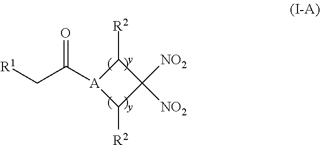
or a pharmaceutically acceptable salt or solvate thereof, wherein:
A is N or C(H);
R¹ is chloro, bromo, —OS(O)₂—(C₁-C₆ alkyl), —OS(O)₂—(C₁-C₆ haloalkyl), —OS(O)₂-(para-methylphenyl), or —OC(O)CF₃;
R² represents independently for each occurrence hydrogen or methyl; and
y represents independently for each occurrence 1 or 2.

16. (canceled)

17. (canceled)

18. (canceled)

19. The method of claim 1, wherein the allosteric modulator of hemoglobin is 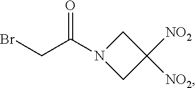 [SMILES: O=C(CBr)N1CC([N+](=O)[O-])([N+](=O)[O-])C1]
or a pharmaceutically acceptable salt or solvate thereof or is selected from the group consisting of: S-nitroso-N-acetylcysteine, S-nitrosocvsteinylglycine, S-nitrosocysteine, S-nitrosohomocysteine, a metal nitrosyl complex, an S-nitro compound, an S-nitroso compound, a thionitrite, a diazeniumdiolate, 4-pyridylmethyl chloride, an alkoxyalkylchloride, dimethoxymethane, N-(hydroxymethyl)acetamide, triphenylmethyl chloride, acetyl chloride, 2-chloroacetic acid, acetic anhydride, a haloacetamide, a haloacetate, benzyl chloride, benzoyl chloride, di-tert-butyl dicarbonate, p-hvdroxyphenacyl bromide, p-acetoxybenzyl chloride, p-methoxvbenzyl chloride, tetrahydropyran, acetamidohydroxymethane, acetone, bis-carboethoxyethene, tert-butoxycarbonyl chloride, alkyl isocyanate, alkoxyalkyl isocyanate, a derivatized dextran, a (polyethylene glycol)-maleimide, 2,4-dinitrophenyl fluoride, and 2,2,2-trichloroethoxycarbonyl.

20. (canceled)

21. The method of claim 1, wherein the inorganic nitrite salt is administered orally at a daily dosage of about 0.1 μg/kg to about 10 mg/kg.

22. (canceled)

23. The method of claim 1, wherein the allosteric modulator of hemoglobin is administered within about 1 hour after administration of the inorganic nitrite salt, and wherein the allosteric modulator of hemoglobin is administered at a dosage sufficient to cause a ten percent increase in the rate at which hemoglobin converts nitrite to nitric oxide in vivo.

24. (canceled)

25. A pharmaceutical composition comprising (i) an inorganic nitrite salt, and (ii) an allosteric modulator of hemoglobin that promotes nitrite reductase activity.

26. The pharmaceutical composition of claim 25, wherein the inorganic nitrite salt is an alkali metal nitrite or is represented by NO₂—N(R′)₄, and wherein R′ represents independently for each occurrence hydrogen, optionally substituted alkyl, optionally substituted heteroalkyl, optionally substituted cycloalkyl, optionally substituted heterocycloalkyl, optionally substituted aryl, optionally substituted aralkyl, optionally substituted heteroaryl, or optionally substituted heteroaralkyl.

27. (canceled)

28. (canceled)

29. The pharmaceutical composition of claim 25, wherein the allosteric modulator of hemoglobin binds to the beta-cysteine-93 residue of hemoglobin.

30. The pharmaceutical composition of claim 25, wherein the allosteric modulator of hemoglobin is a compound of Formula I or II, wherein Formula I is represented by: 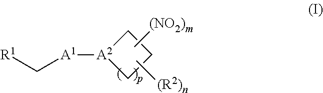
or a pharmaceutically acceptable salt or solvate thereof, wherein:
A¹ is —C(O)— or —(C(R³)₂)ₓC(O)(C(R³)₂)ₓ—;
A² is N or —C(R⁴)—;
R¹ is halogen, —OS(O)₂R⁵, or —OC(O)CF₃;
R² is C₁-C₆ alkyl;
R³ and R⁴ each represent independently for each occurrence hydrogen or C₁-C₅ alkyl;
R⁵ is C₁-C₆ alkyl, C₁-C₆ haloalkyl, aryl, or aralkyl;
m and p are independently 1, 2, or 3; and
n and x each represent independently for each occurrence 0, 1, 2, or 3; and
Formula II is represented by: 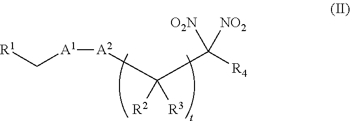
or a pharmaceutically acceptable salt or solvate thereof: wherein:
A¹ is —C(O)— or —(C(R⁵)₂)ₓC(O)(C(R⁵)₂)ₓ—;
A² is —N(R⁵)— or —C(R²)(R³)—;
R¹ is halogen, —OS(O)₂R⁶, or —OC(O)CF₃;
R² and R³ each represent independently for each occurrence hydrogen or C₁-C₆ alkyl; or
R² and R³ are taken together with the carbon atom to which they are attached to form a 3-6 membered, saturated carbocyclic ring;
R⁴ is hydrogen or C₁-C₆ alkyl;
R⁵ represents independently for each occurrence hydrogen or C₁-C₆ alkyl;
R⁶ is C₁-C₆ alkyl, C₁-C₆ haloalkyl, aryl, or aralkyl;
t is an integer in the range from 1 to 12; and
x represents independently for each occurrence 0, 1, 2, or 3.

31. The pharmaceutical composition of claim 25, wherein the allosteric modulator of hemoglobin is a compound of Formula I-A: 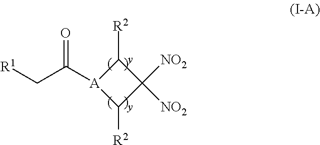
or a pharmaceutically acceptable salt or solvate thereof, wherein:
A is N or C(H);
R¹ is chloro, bromo, —OS(O)₂—(C₁-C₆ alkyl), —OS(O)₂—(C₁-C₆ haloalkyl), —OS(O)₂-(para-methylphenyl), or —OC(O)CF₃;
R² represents independently for each occurrence hydrogen or methyl; and
y represents independently for each occurrence 1 or 2.

32. (canceled)

33. (canceled)

34. (canceled)

35. The pharmaceutical composition of claim 25, wherein the allosteric modulator of hemoglobin is 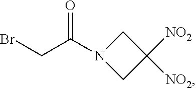 [SMILES: O=C(CBr)N1CC([N+](=O)[O-])([N+](=O)[O-])C1]
or a pharmaceutically acceptable salt or solvate thereof or is selected from the group consisting of: S-nitroso-N-acetylcysteine, S-nitrosocysteinylglycine, S-nitrosocysteine, S-nitrosohomocysteine, a metal nitrosyl complex, an S-nitro compound, an S-nitroso compound, a thionitrite, a diazeniumdiolate, 4-pyridylmethyl chloride, an alkoxyalkylchloride, dimethoxymethane, N-(hydroxymethyl)acetamide, triphenylmethyl chloride, acetyl chloride, 2-chloroacetic acid, acetic anhydride, a haloacetamide, a haloacetate, benzyl chloride, benzoyl chloride, di-tert-butyl dicarbonate, p-hydroxyphenacyl bromide, p-acetoxybenzyl chloride, p-methoxybenzyl chloride, tetrahydropyran, acetamidohydroxymethane, acetone, bis-carboethoxyethene, tert-butoxycarbonyl chloride, alkyl isocyanate, alkoxyalkyl isocyanate, a derivatized dextran, a (polyethylene glycol)-maleimide, 2,4-dinitrophenyl fluoride, and 2,2,2-trichloroethoxycarbonyl.

36. (canceled)

37. (canceled)

38. A method of treating a patient suffering from reduced blood volume or anemia, the method comprising:
administering to a patient in need thereof a blood product by injection and a therapeutic agent selected from the group consisting of; an organonitro compound of Formula I, organonitro compound of Formula II, hemoglobin conjugate of Formula III, hemoglobin conjugate of Formula IV, and an erythrocyte cell that has been exposed to an organonitro compound of Formula I or II; wherein Formula I is represented by: 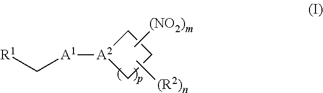
or a pharmaceutically acceptable salt or solvate thereof, wherein:
A¹ is —C(O)— or —(C(R³)₂)ₓC(O)(C(R³)₂)ₓ;
A² is N or —C(R⁴)—;
R¹ is halogen, —OS(O)₂R⁵, or —OC(O)CF₃;
R² is C₁-C₆ alkyl;
R³ and R⁴ each represent independently for each occurrence hydrogen or C₁-C₅ alkyl;
R⁵ is C₁-C₆ alkyl, C₁-C₆ haloalkyl, aryl, or aralkyl;
m and p are independently 1, 2, or 3; and
n and x each represent independently for each occurrence 0, 1, 2, or 3;
Formula II is represented by: 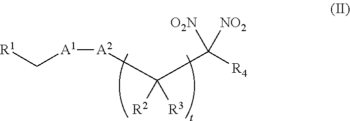
or a pharmaceutically acceptable salt or solvate thereof: wherein:
A¹ is —C(O)— or —(C(R⁵)₂)ₓC(O)(C(R⁵)₂)ₓ—;
A² is —N(R⁵)— or —C(R²)(R³)—;
R¹ is halogen, —OS(O)₂R⁶, or —OC(O)CF₃;
R² and R³ each represent independently for each occurrence hydrogen or C₁-C₆ alkyl; or
R² and R³ are taken together with the carbon atom to which they are attached to form a 3-6 membered, saturated carbocyclic ring;
R⁴ is hydrogen or C₁-C₆ alkyl;
R⁵ represents independently for each occurrence hydrogen or C₁-C₆ alkyl;
R⁶ is C₁-C₆ alkyl, C₁-C₆ haloalkyl, aryl, or aralkyl;
t is an integer in the range from 1 to 12; and
x represents independently for each occurrence 0, 1, 2, or 3;
Formula III is represented by: 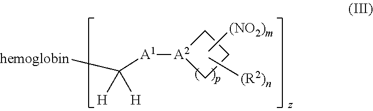
or a pharmaceutically acceptable salt or solvate thereof, wherein:
A¹ is —C(O)— or —C(O)(C(R³)₂)ₓ—;
A² is N or —C(R⁴)—;
R² is C₁-C₆ alkyl;
R³ and R⁴ each represent independently for each occurrence hydrogen or C₁-C₅ alkyl;
m and p are independently 1, 2, or 3;
n is 0, 1, 2, or 3;
x is 1, 2, or 3; and
z is an integer from 1 to 10; and
Formula IV is represented by: 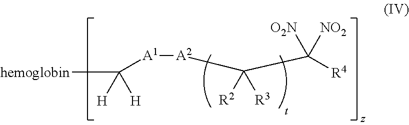
or a pharmaceutically acceptable salt or solvate thereof, wherein:
A¹ is —C(O)— or —C(O)(C(R⁵)₂)ₓ—;
A² is —N(R⁵)— or —C(R²)(R³)—;
R² and R³ each represent independently for each occurrence hydrogen or C₁-C₆ alkyl; or
R² and R³ are taken together with the carbon atom to which they are attached to form a 3-6 membered, saturated carbocyclic ring;
R⁴ is hydrogen or C₁-C₆ alkyl;
R⁵ represents independently for each occurrence hydrogen or C₁-C₆ alkyl;
t is an integer in the range from 1 to 12;
x is 1, 2, or 3; and
z is an integer from 1 to 10.

39. (canceled)

40. (canceled)

41. The method of claim 38, wherein the blood product comprises erythrocyte cells or blood plasma.

42. (canceled)

43. (canceled)

44. (canceled)

45. (canceled)

46. The method of claim 38, further comprising administering an alkali metal nitrite to the patient.

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)

76. (canceled)

77. (canceled)

78. (canceled)

79. (canceled)

80. (canceled)

81. (canceled)

82. (canceled)

83. (canceled)

84. The pharmaceutical composition of claim 25, wherein the pharmaceutical composition further comprises (iii) a blood product, wherein the blood product is whole blood or comprises at least one of erythrocyte cells and blood plasma.

85. (canceled)

86. (canceled)

87. (canceled)

88. (canceled)

89. (canceled)

90. (canceled)

91. (canceled)

92. (canceled)

93. (canceled)

94. (canceled)

95. (canceled)

96. (canceled)

97. (canceled)

98. (canceled)

99. (canceled)

100. (canceled)

101. (canceled)

102. (canceled)

103. (canceled)

104. (canceled)

105. (canceled)

106. (canceled)
